SEQUENCE LISTING (44 sequences)

SEQ ID NO: 1 - DNA, 64 bp, Artificial Sequence
  Description of Artificial Sequence Note = Synthetic Construct
gatccccaggcagtgcgatcctccgtttcaagagatatccggtaatctccaaattttttggaaa

SEQ ID NO: 2 - DNA, 64 bp, Artificial Sequence
  Description of Artificial Sequence Note = Synthetic Construct
agcttttccaaaaaatttggagattaccggatatctcttgaaacggaggatcgcactgcctggg

SEQ ID NO: 3 - DNA, 19 bp, Artificial Sequence
  Description of Artificial Sequence Note = Synthetic Construct
ctggcagaccagcctgtgt

SEQ ID NO: 4 - DNA, 19 bp, Artificial Sequence
  Description of Artificial Sequence NOte = Synthetic Construct
gtggcagctgacatgaatg

SEQ ID NO: 5 - DNA, 19 bp, Artificial Sequence
  Description of Artificial Sequence Note = Synthetic Construct
gctcaactcagcacacact

SEQ ID NO: 6 - DNA, 19 bp, Artificial Sequence
  Description of Artificial Sequence Note = Synthetic Construct
gttgcagctgatatgaatg

SEQ ID NO: 7 - DNA, 19 bp, Artificial Sequence
  Description of Artificial Sequence Note = Synthetic Construct
gctcttaccgaagcaacaa

SEQ ID NO: 8 - DNA, 19 bp, Artificial Sequence
  Description of Artificial Sequence Note = Synthetic Construct
ggagactgaagttgtatgt

SEQ ID NO: 9 - DNA, 19 bp, Artificial Sequence
  Description of Artificial Sequence Note = Synthetic Construct
gcagactgaaagtttaaga

SEQ ID NO: 10 - DNA, 21 bp, Artificial Sequence
  Description of Artificial Sequence Note = Synthetic Construct
cgaatgtaatgtgaatggaat

SEQ ID NO: 11 - DNA, 21 bp, Artificial Sequence
  Description of Artificial Sequence Note = Synthetic Construct
tgttgcagcttcctacttcaa

SEQ ID NO: 12 - DNA, 21 bp, Artificial Sequence
  Description of Artificial Sequence Note = Synthetic Construct
gctcaggcaccaacagttatc

SEQ ID NO: 13 - DNA, 20 bp, Artificial Sequence
  Description of Artificial Sequence Note = Synthetic Construct
gctgccacttgacatccaag

SEQ ID NO: 14 - DNA, 20 bp, Artificial Sequence
  Description of Artificial Sequence Note = Synthetic Construct
gtgccttgggtgtcaagttg

SEQ ID NO: 15 - DNA, 19 bp, Artificial Sequence
  Description of Artificial Sequence Note = Synthetic Construct
ccagggatgccatatcggg

SEQ ID NO: 16 - DNA, 22 bp, Artificial Sequence
  Description of Artificial Sequence Note = Synthetic Construct
agcttgtggtaagacatgcttg

SEQ ID NO: 17 - DNA, 23 bp, Artificial Sequence
  Description of Artificial Sequence Note = Synthetic Construct
gtgtcccataaagccaactctac

SEQ ID NO: 18 - DNA, 20 bp, Artificial Sequence
  Description of Artificial Sequence Note = Synthetic Construct
accacagtccatgccatcac

SEQ ID NO: 19 - DNA, 20 bp, Artificial Sequence
  Description of Artificial Sequence Note = Synthetic Construct
tccaccaccctgttgctgta

SEQ ID NO: 20 - DNA, 29 bp, Artificial Sequence
  Description of Artificial Sequence Note = Synthetic Construct
cgcggatccaccatggctcaggcaccaac

SEQ ID NO: 21 - DNA, 33 bp, Artificial Sequence
  Description of Artificial Sequence Note = Synthetic Construct
cgcgtcgacctagaaagcgttcatggctctcct

SEQ ID NO: 22 - DNA, 29 bp, Artificial Sequence
  Description of Artificial Sequence Note = Synthetic Construct
cgcagatctaccatgccgtccgagaagac

SEQ ID NO: 23 - DNA, 31 bp, Artificial Sequence
  Description of Artificial Sequence Note = Synthetic Construct
cgcgtcgacctacacagccattgctgtcccg

SEQ ID NO: 24 - DNA, 28 bp, Artificial Sequence
  Description of Artificial Sequence Note = Synthetic Construct
cgcaccggtccaccatggtgagcaaggg

SEQ ID NO: 25 - DNA, 30 bp, Artificial Sequence
  Description of Artificial Sequence Note = Synthetic Construct
cgccgtacgtacacagccattgctgtcccg

SEQ ID NO: 26 - DNA, 36 bp, Artificial Sequence
  Description of Artificial Sequence Note = Synthetic Construct
cgctgatcaaccatgacctctaggaagaaagtgttg

SEQ ID NO: 27 - DNA, 30 bp, Artificial Sequence
  Description of Artificial Sequence Note = Synthetic Construct
cgcgtcgacctaacaactgcagctttctgc

SEQ ID NO: 28 - DNA, 35 bp, Artificial Sequence
  Description of Artificial Sequence Note = Synthetic Construct
cgcggatccaccatgtcggaagattcagaggttgt

SEQ ID NO: 29 - DNA, 30 bp, Artificial Sequence
  Description of Artificial Sequence Note = Synthetic Construct
cgcgtcgacctatccccatgcctgggattt

SEQ ID NO: 30 - DNA, 33 bp, Artificial Sequence
  Description of Artificial Sequence Note = Synthetic Construct
cggactagtccaccatggactacaaagaccatg

SEQ ID NO: 31 - DNA, 46 bp, Artificial Sequence
  Description of Artificial Sequence Note = Synthetic Construct
gtcttggatgtcaggtcgccgcggacatgaacgagtgttgtctgtg

SEQ ID NO: 32 - DNA, 46 bp, Artificial Sequence
  Description of Artificial Sequence Note = Synthetic Construct
cacagacaacactcgttcatgtccgcggcgacctgacatccaagac

SEQ ID NO: 33 - DNA, 36 bp, Artificial Sequence
  Description of Artificial Sequence Note = Synthetic Construct
ctctggtgttggaaagaactctctcatgaaccagta

SEQ ID NO: 34 - DNA, 36 bp, Artificial Sequence
  Description of Artificial Sequence Note = Synthetic Construct
tactggttcatgagagagttctttccaacaccagag

SEQ ID NO: 35 - DNA, 28 bp, Artificial Sequence
  Description of Artificial Sequence Note = Synthetic Construct
ggacacagccggtctagaacggttccag

SEQ ID NO: 36 - DNA, 28 bp, Artificial Sequence
  Description of Artificial Sequence Note = Synthetic Construct
ctggaaccgttctagaccggctgtgtcc

SEQ ID NO: 37 - DNA, 45 bp, Artificial Sequence
  Description of Artificial Sequence Note = Synthetic Construct
ggagagtctgctgttggcaaaaacagcctggttcttcgctttgtg

SEQ ID NO: 38 - DNA, 45 bp, Artificial Sequence
  Description of Artificial Sequence Note = Synthetic Construct
cacaaagcgaagaaccaggctgtttttgccaacagcagactctcc

SEQ ID NO: 39 - DNA, 88 bp, Artificial Sequence
  Description of Artificial Sequence Note = Synthetic Construct
gatcaccatggactacaaagaccatgacggtgattataaagatcatgacatcgactacaaggatgacgatgacaagggatccagcaca

SEQ ID NO: 40 - DNA, 80 bp, Artificial Sequence
  Description of Artificial Sequence Note = Synthetic Construct
ctggatcccttgtcatcgtcatccttgtagtcgatgtcatgatctttataatcaccgtcatggtctttgtagtccatggt

SEQ ID NO: 41 - DNA, 47 bp, Artificial Sequence
  Description of Artificial Sequence Note = Synthetic Construct
gatcaccatgggatacccatacgacgtcccagattacgccactggag

SEQ ID NO: 42 - DNA, 47 bp, Artificial Sequence; identical to SEQ ID NO: 41
  Description of Artificial Sequence Note = Synthetic Construct

SEQ ID NO: 43 - DNA, 339 bp, Artificial Sequence
  Description of Artificial Sequence Note = Synthetic Construct
atggctcaggcaccaacagttatcgtgactcaacctggattcgttcgtgctccccaaaattccaactggcagaccagcctgtgtgattgcttcagtgactgcggagtctgcctctgtgggaccttttgtttcacttgtcttggatgtcaagtggcagctgacatgaatgagtgttgtctgtgtggaacaacggtggccatgaggactctctaccgaacccgatacggcattcctggatctatttgtgatgactacatggtcacactcttctgtcctgtttgctctgtgtgccaactcaagagagacattaacaggaggagagccatgaacgctttctaa

SEQ ID NO: 44 - DNA, 339 bp, Artificial Sequence
  Description of Artificial Sequence Note = Synthetic Construct
atggctcaggcaccaacagttatcgtgactcaacctggattcgttcgtgctccccaaaattccaactggcagaccagcctgtgtgattgcttcagtgactgcggagtctgcctctgtgggaccttttgtttcacttgtcttggatgtcaagtggcagctgacatgaatgagtgttgtctgtgtggaacaacggtggccatgaggactctctaccgaacccgatacggcattcctggatctatttgtgatgactacatggccgcagccgccgctcctgtttgctctgtgtgccaactcaagagagacattaacaggaggagagccatgaacgctttctaa

(19) United States
(12) Patent Application Publication
(10) Pub. No.: US 2013/0184223 A1
(43) Pub. Date: Jul. 18, 2013

(54) METHODS AND COMPOSITIONS RELATED TO MODULATING AUTOPHAGY

(71) Applicants: Hartmut Land, Rochester, NY (US); Conan Kinsey, Rochester, NY (US)

(72) Inventors: Hartmut Land, Rochester, NY (US); Conan Kinsey, Rochester, NY (US)

(73) Assignee: UNIVERSITY OF ROCHESTER, Rochester, NY (US)

(21) Appl. No.: 13/698,521

(22) PCT Filed: May 20, 2011
(86) PCT No.: PCT/US11/37406

§ 371 Date: Mar. 5, 2013

Related U.S. Application Data
(60) Provisional application No. 61/346,867, filed on May 20, 2010.

(51) Int. Cl. A61K 38/16; C12Q 1/02; A61K 31/7088; C12Q 1/68

(52) U.S. Cl. CPC A61K 38/16; C12Q 1/68; C12Q 1/02; A61K 31/7088
USPC 514/19.4; 435/6.12; 435/6.14; 435/29; 514/19.3; 514/19.5; 514/19.6; 514/19.8; 514/44 R; 514/44 A

(57) ABSTRACT

Disclosed are compositions and methods related to new targets for cancer treatment the modulation of autophagy.

### I. BACKGROUND

Understanding the molecular underpinnings of cancer is of critical importance to developing targeted intervention strategies. Identification of such targets, however, is notoriously difficult and unpredictable. Malignant cell transformation requires the cooperation of a few oncogenic mutations that cause substantial reorganization of many cell features (Hanahan, D. & Weinberg, R. A. (2000) Cell 100, 57-70) and induce complex changes in gene expression patterns (Yu, J. et al. (1999) Proc Natl Acad Sci USA 96, 14517-22 (1999); Zhao, R. et al. (2000) Genes Dev 14, 981-93; Schulze, A., et al. (2000) Genes Dev 15, 981-94; Huang, E. et al. (2003) Nat Genet. 34, 226-30; Boiko, A. D. et al. A (2006) Genes Dev 20, 236-52). Genes critical to this multi-faceted cellular phenotype thus only have been identified following signaling pathway analysis (Vogelstein, B., et al. (2000) Nature 408, 307-10; Vousden, K. H. & Lu, X. (2002) Nat Rev Cancer 2, 594-604; Downward, J. (2003) Nat Rev Cancer 3, 11-22; Rodriguez-Viciana, P. et al. (2005) Cold Spring Harb Symp Quant Biol 70, 461-7) or on an ad hoc basis (Schulze, A., et al. (2000) Genes Dev 15, 981-94; Okada, F. et al. (1998) Proc Natl Acad Sci USA 95, 3609-14; Clark, E. A., et al. (2000) Nature 406, 532-5; Yang, J. et al. (2004) Cell 117, 927-39; Minn, A. J. et al. (2005) Nature 436, 518-24). Thus, there is a need for new methods of identifying genes critical to the formation, proliferation and maintenance of cancer.

### II. SUMMARY

Disclosed are methods and compositions related to in one aspect methods for identifying targets for the treatment of a cancer. In other aspect, disclosed herein are methods for screening for an agent that treats a cancer. Also disclosed herein are methods of treating cancer. Further disclosed are methods related to determining whether a cancer is susceptible to treatment.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1.1 shows the Protein Consensus Alignment of Vertebrate Plac8 Proteins and Eukaryotic Proteins Containing the Plac8 Family Domain

FIG. 1.2 shows The Autophagy Cascade and Specific Inhibitors of Different Autophagy Phases.

FIG. 2.1 shows that Plac8 is synergistically up-regulated by mp53 and Ras. (A) Total RNA was extracted from YAMC, Vector Control, Single Oncogene (mp53 or Ras) and Transformed cells (expressing both mp53 and Ras). Expression of Plac8 was measured via real-time PCR using an iCycler. The induction of plac8 expression from YAMC to mp53, RasV12 and Transformed cells is 2.3, 9.4, and 23.8 fold respectively. Plac8 responds to both oncogenic mutations alone, most notably RasV12, but is further up-regulated cooperatively in the transformed state. (B) Plac8 Polysomal RNA expression values derived from the micro-array analysis of from YAMC, Vector Control, Single Oncogene (mp53 or Ras) and Transformed cells (expressing both mp53 and Ras) normalized to YAMC (McMurray, et al., 2008). The induction of plac8 expression from YAMC to mp53, RasV12 and Transformed cells is 0.7, 2.3, and 4.0 fold respectively.

FIG. 2.2 shows that Plac8 KD inhibits tumor formation of mp53/Ras cells. (A) Transformed cells were infected with vector control or one of the three Plac8 shRNA targeting constructs. A polyclonal population of cells stably expressing the integrated shRNA constructs were selected via puromycin. Confirmation of knock-down was validated by real-time RT-PCR analysis. The shPlac8 155, 240 and 461 siRNA constructs can knock down Plac8 expression levels to 76%, 99% and 92% of vector control levels respectively. (B) Vector control and Plac8 KD cell lines were injected into nude mice and tumor volume was measure weekly for 4 weeks. Plac8 KD cells show a significant inhibition in tumor formation compared to vector control. Number of injections (n) and significance levels as compared to matched controls are indicated; ***P<0.001.

FIG. 2.3 shows that Plac8 KD inhibition of tumor formation can be rescued by reexpression of a shRNA resistant form of Plac8. (A) Plac8 cDNA was PCR cloned and five silent mutations introduced via site directed mutagenesis in the 19 nt targeting region of the shPlac8 240 construct. The Plac8 cDNA was cloned into the pBabe retroviral expression vector with a HA tag on the N-terminus. This was introduced into shPlac8 240 infected cells where Plac8 had been successfully knocked-down. Confirmation of knock-down, over-expression and rescue was validated by real-time quantitative PCR analysis. (B) Immunoblotting for the HA epitope reveals a 15 kDa protein, which is the predicted size for the HA tagged Plac8 protein. (C) The vector control, Plac8 over-expression, Plac8 shRNA knock down, and Plac8 knock-down with rescue shRNA resistant Plac8 cells were injected into nude mice at 5×105 cells per injection. The mice were measured every week for tumor burden starting after 2 weeks post injection, and ending after 4 weeks. The Plac8 knock down cells show no tumors by 4 weeks, where as the vector control, plac8 over-expressing, and plac8 rescue cell lines form tumors. Number of injections (n) and significance levels as compared to matched controls are indicated; ***P<0.001.

FIG. 2.4 shows that Plac8 KD or over-expression has no effect on p53 or Ras protein levels in mp53/Ras transformed cells. (A) Cell protein lysates were harvested from vector control, Plac8 overexpression, Plac8 sh240 knock down, and plac8 knock-down with rescue plac8 mutant cells and immunoblotted for p53, Ras and beta-tubulin. Plac8 overexpression or knock down do not affect p53 or Ras protein levels in mp53/Ras transformed cells. (B) Cell protein lysates were harvested from vector control and HA Plac8 over-expression Ras cells and immunoblotted for p53, HA-tagged Plac8 and beta-tubulin. Plac8 over-expression does not perturb WT p53 protein levels.

FIG. 2.5 shows that Plac8 KD in HT-29 human colorectal adenocarcinoma cells inhibits tumor formation (A) HT-29 cells were infected with vector control or Plac8 shRNA targeting construct. A polyclonal population of cells stably expressing the integrated shRNA constructs were selected via puromycin. Confirmation of knock down was validated by real-time RT-PCR analysis. The shPlac8 shRNA construct can knock down Plac8 expression levels 90% of vector control levels. (B) Plac8 knock down HT-29 cells lines and vector control were injected into nude mice at 1.25×105 cells per injection. The mice were measured every week for tumor burden starting after 2 weeks post injection, and ending after 4 weeks. Plac8 KD HT-29 cells do not grow tumors after four weeks. Number of injections (n) and significance levels as compared to matched controls are indicated; ***P<0.001.

FIG. 2.6 shows that Plac8 KD in Panc10.05 and PANC-1 pancreatic adenocarcinoma cells inhibits tumor formation. (A, B) Plac8 total mRNA expression levels from vector control and Plac8 shRNA KD Panc10.05, CAPAN-2, and Panc-1 cell line cDNA generated from reverse transcription of total RNA samples were quantified using a quantitative Real-time PCR iCycler (Bio-rad) and analyzed via the ΔΔCt method to generate relative fold expression values normalized to GAPDH and then to Vector control. (C,D) Vector control and Plac8 KD Panc10.05 and Panc-1 cell lines were injected into NOD/SCID mice at 1×106 and 2.5×106 cells per injection respectively and tumor volume was measure weekly for 4 weeks. Both pancreatic adenocarcinoma cell lines Plac8 inhibits tumor formation. Each cell line was injected twelve times and significance levels are P<0.001 for Plac8 KD cells compared to matched vector controls.

FIG. 2.7 shows that Plac8 KD in CAPAN-2 cells inhibits tumor formation, which can be rescued by expression of a shRNA resistant form of Plac8 in CAPAN-2 cells. (A) Plac8 total mRNA expression levels from vector control and Plac8 shRNA KD CAPAN-2 cell line cDNA generated from reverse transcription of total RNA samples were quantified using a quantitative Real-time PCR iCycler (Bio-Rad) and analyzed via the ΔΔCt method to generate relative fold expression values normalized to GAPDH and then to Vector control. (B) 3× Flag-tagged shRNA resistant Plac8 was stably expressed in vector control and Plac8 shRNA KD CAPAN-2 cells via lentiviral infection. The generated cell line protein lysates were immunoblotted for 3× Flag-tag and to control for protein loading beta-Tubulin. 3× Flag-tagged Plac8 expressing cells show a specific protein band around 18 kDa, the predicted size for the 3× Flag-tagged Plac8 protein. (C) The vector control, Plac8 over-expression, Plac8 shRNA knock-down, and Plac8 knock-down with rescue shRNA resistant Plac8 cells were injected into nude mice at 5×105 cells per injection. The mice were measured every week for tumor burden starting after 2 weeks post injection, and ending after 4 weeks. The Plac8 knock-down cells show no tumors by 4 weeks, where as the vector control, Plac8 over-expressing, and Plac8 rescue cell lines form tumors. Each cell line was injected twelve times and significance levels are P<0.001 for Plac8 KD cells compared to matched vector controls, Plac8 over-expression, and Plac8 rescue cells.

FIG. 3.1 shows that Plac8 c-terminal polyclonal antibody recognizes a 13 kDa protein in both murine and human cells. (A) Immunoblotting Vector, Plac8 shRNA KD, and exogenous 3× Flag-tagged Plac8 expressing mp53/Ras transformed cells with anti-Plac8 antibody recognizes a 13 kDa protein that is diminished with Plac8 shRNA KD and recognizes a higher band in the 3× Flag Plac8 lane, which corresponds to the exogenous 3× Flag-tagged Plac8 protein. (B) Immunoblotting of murine YAMC, vector, mp53, Ras, and mp53/Ras transformed cells with anti-Plac8 antibody shows a cooperative increase in Plac8 protein level similar to what we have observed in the Plac8 polysomal RNA profile. (C,D,E,F) Immunoblotting of Vector and Plac8 shRNA KD human HT-29 colorectal adenocarcinoma and human CAPAN-2, PANC-1, and Panc10.05

FIG. 3.2 shows that Plac8 protein localizes to lysosomes. (A) mp53/Ras transformed murine vector control and Plac8 shRNA-mediated KD cells were fixed and immunofluoresently stained using anti-Plac8 and anti-Lamp2 antibodies with appropriate secondary antibodies conjugated to Alexa488 (green) and Alexa555 (red) respectively, then imaged using confocal microscopy. Plac8 sub-cellular localization shows a punctate distribution and partially co-stains with Lamp2, a lysosomal protein, indicative of lysosomal localization. (B) Sub-cellular fractionationalion and immunoblotting of mp53/Ras transformed murine cells for Plac8, known lysosomal proteins Rab7 and Lamp2, autophagosomal protein LC3, and a cytosolic control RhoA. Lanes are as follows; WC(1): whole cell lysate, N(2): nuclear fraction, C(3): cytosolic fraction, CL(4): crude lysosomal fraction, M(5): microsomal fraction, L(6): lysosomal fraction. Plac8, Rab7, Lamp2, and LC3-II enrich in the lysosomal fractions, indicating that Plac8 is a lysosomal protein. LC3-I and RhoA enrich in the cytosolic fraction.

FIG. 3.3 shows that Plac8 is an internal lysosomal protein. Cytosolic fractions (C) and crude lysosomal fractions (CL) were isolated from mp53/Ras transformed murine cells and the crude lysosomal fraction subjected to a Proteinase K protection assay. In short, lysosomes are treated with Proteinase K for 30 min at 37° C. and immunoblotted. If proteins are inside the lysosome they are protected from degradation. Rab7 a known external lysosomal protein is degraded, were as, known internal lysosomal proteins Lamp2 and Cathepsin D are protected from degradation. Plac8 is also protected from degradation indicating Plac8 is an internal lysosomal protein. Triton-X is added to another sample to dissolve the protective membrane to control for degradation.

FIG. 3.4 shows that Plac8 protein levels are increased around areas of tumor necrosis, under nutrient stress, and hypoxic conditions. (A) mp53/Ras transformed murine cells stably expressing GFP were injected intra-dermally into nude mice and allowed to grow for 4 weeks. The mice were sacrificed, and the tumors were removed and cryosectioned. The tumor sections were then fixed and immunofluorescently stained with anti-Plac8 antibody and appropriate secondary conjugated to Alexa555 (red). Cells were also stained with a nuclear stain Topro3 and imaged via confocal microscopy. The necrotic region is indicated by the dashed line and the large letter N. Plac8 staining is increased around areas of low nuclear density, indicating necrosis. (B) mp53/Ras transformed murine cells were subjected to nutrient starvation (NS) by exposure to Hank's Buffered Saline Solution for 1 hr. and hypoxia (Hypox.) by placing cells in a sealed chamber and flooding it with nitrogen for 5 minutes to evacuate the oxygen then allowing the cells to grow for 24 hrs. Cell protein was harvested and immunoblotted for Plac8 and beta-Tubulin. The Plac8 protein is increased under nutrient starvation and hypoxic conditions.

FIG. 3.5 shows that Plac8 KD results in an accumulation of autophagosomes. Vector control and Plac8 shRNA KD mp53/Ras transformed cells were fixed and analyzed via transmission electron microscopy. Plac8 shRNA KD cells show an increase in the number of autophagosomal structures as highlighted by the black arrows. The magnified inset of one of these structures found in Plac8 shRNA KD cells shows the presence of ribosomes inside the structure, which are specific to autophagosomes.

FIG. 3.6 shows that Plac8 KD results in an accumulation of autophagosomal markers. (A, B, C) Vector control and Plac8 shRNA KD mp53/Ras transformed, CAPAN-2, and HT-29 cells were nutrient starved by treatment with HBSS for 0, 15, 30 and 60 minutes. Protein lysates for cells were harvested and immunoblotted for p62, LC3, beta-Tubulin and Plac8 (for mp53/Ras and CAPAN-2 cells). Vector control cells show a decline in p62 protein, a conversion of LC3-I to LC3-II, a decrease in LC3-II protein, and an increase in Plac8 over time under nutrient starvation, indicating autophagic activity. Plac8 shRNA KD cells show an accumulation of p62, LC3-I, and LC3-II, indicating a block in the autophagy process. (D,E) Plac8 shRNA KD accumulation of the autophagic markers p62, LC3-I, and LC3-II can be rescued by expressing an shRNA resistant 3× Flag-tagged Plac8 in mp53/Ras transformed and CAPAN-2 cells.

FIG. 3.7 shows that Plac8 KD inhibits autophagosomal/lysosomal fusion. (A,B) GFP-LC3 expressing vector, Plac8 shRNA KD, and Plac8 shRNA KD with exogenous shRNA resistant Plac8 mp53/Ras transformed and CAPAN-2 cells were nutrients starved in HBSS for 15 minutes, fixed and immunofluorescently stained for Lamp2. Cells were imaged by confocal microscopy. Images were analyzed with ImageJ to highlight and quantify colocalization. (C,D) Colocalization is inhibited in Plac8 shRNA KD by 82% in mp53/Ras cells and 60% in CAPAN-2 cells. The colocalization inhibition by Plac8 KD is rescued with expression of the shRNA resistant Plac8. n>50 cells for all cell lines; P<0.001 for shPlac8/Vector vs. Vector/Vector or shPlac8/shR-Plac8 for both mp53/Ras transformed and CAPAN-2 cells.

FIG. 3.8 shows that Rab7 DN expression inhibits tumor formation and results in an accumulation of autophagosomal markers. (A,C) 3× Flag-tagged Rab7 DN (Rab7T22N) was expressed in mp53/Ras transformed cells and CAPAN-2 cells and vector control and Rab7 DN expressing cells were injected into nude mice at 5×105 cells per injection. The mice were measured every week for tumor burden starting after 2 weeks post injection, and ending after 4 weeks for mp53/Ras transformed cells and 5 weeks for CAPAN-2 cells. Vector n=12, Rab7 DN n=10 for mp53/Ras transformed cells (A) and Vector n=12, Rab7 n=12 for CAPAN-2 cells (C) and significance levels are P<0.001 for Rab7 DN cells compared to matched vector controls. Vector control and 3× Flag-tagged Rab7 DN expressing mp53/Ras transformed cell (B) and CAPAN-2 (D) cell line protein lysates were immunoblotted for p62, LC3, Rab7 and beta-Tubulin.

FIG. 3.9 shows that Rab7 DA can rescue Plac8 KD tumor formation and accumulation of autophagosomal markers. (A,B) The vector control, 3× Flag-tagged Rab7 DA expression, Plac8 shRNA knock down, and plac8 knock-down with 3× Flag-tagged Rab7 DA expressing cells were injected into nude mice at 5×105 cells per injection. The mice were measured every week for tumor burden starting after 2 weeks post injection, and ending after 4 weeks for mp53/Ras transformed cells and 5 weeks for CAPAN-2 cells. Each cell line was injected twelve times and significance levels are P<0.001 for Plac8 KD cells compared to matched vector controls, Rab7 DA expressing, and Plac8 KD with Rab7 DA expression cells. Significance levels are P<0.01 for Rab7 DA expressing cells compared to matched vector controls and Plac8 KD with Rab7 DA expression cells. (C,D) The vector control, 3× Flagtagged Rab7 DA expression, Plac8 shRNA knock down, and Plac8 knock-down with 3× Flag-tagged Rab7 DA expressing protein lysates were immunoblotted for p62, LC3, Rab7, Plac8 and beta-Tubulin.

FIG. 3.10 shows that Plac8 KD inhibition of autophagosomal/lysosomal fusion can be rescued by Rab7 DA and phenocopies Rab7 DN. (A,B) GFP-LC3 expressing vector control, 3× Flag-tagged Rab7 DA expression, Plac8 shRNA knock-down, and plac8 knock-down with 3× Flag-tagged Rab7 DA expressing mp53/Ras transformed and CAPAN-2 cells and 3× Flag-tagged Rab7 DN expressing mp53/Ras transformed cells were nutrients starved in HBSS for 15 minutes, fixed and immunofluorescently stained for Lamp2. Cells were imaged via confocal microscopy. Images were analyzed with ImageJ to highlight and quantify colocalization. (C,D) Colocalization is inhibited in Plac8 shRNA KD by 82% in mp53/Ras cells and 60% in CAPAN-2 cells. The colocalization inhibition by Plac8 KD is rescued with expression of the 3× Flag-tagged Rab7 DA mutant. n>50 cells for all cell lines; Significance levels are P<0.001 for shPlac8/Vector vs. Vector/Vector, Vector/Rab7 DA, or shPlac8/Rab7 DA for both mp53/Ras transformed and CAPAN-2 cells. Colocalization is inhibited by 3× Flag-tagged Rab7 DN expression by 74% in mp53/Ras transformed cells. n>50 cells for all cell lines; Significance levels are P<0.001 for Vector/Rab7 DN vs. Vector/Vector.

FIG. 3.11 shows that Rab5a DN expression does not inhibit tumor formation. (A, C) 3× Flag-tagged Rab5a DN (Rab5aS34N) was expressed in mp53/Ras transformed cells and CAPAN-2 cells and vector control and Rab5a DN expressing cells were injected into nude mice at 5×105 cells per injection. The mice were measured every week for tumor burden starting after 2 weeks post injection, and ending after 4 weeks for mp53/Ras transformed cells and 5 weeks for CAPAN-2 cells. Vector n=12, Rab5a DN n=12; significance levels are P<0.05 for Rab5a DN vs. Vector control for mp53/Ras transformed cell lines and not statistically significant for CAPAN-2 cells. Vector control and 3× Flag-tagged Rab5a DN expressing mp53/Ras transformed cell (B) and CAPAN-2 (D) cell line protein lysates were immunoblotted for p62, LC3, Rab5 and beta-Tubulin.

FIG. 3.12 show that Rab5a DN expression inhibits endocytosis of Alexa488 labeled dextran. Vector control and Rab5a DN expressing mp53/Ras transformed (A) and CAPAN-2 cells (C) were treated with Alexa 488-Dextran, fixed with paraformaldehyde, stained with the nuclear stain Topro 3, and imaged with confocal microscopy. Vector control and Rab5a DN expressing mp53/Ras transformed (B) and CAPAN-2 (D) cells were treated with Alexa 488-Dextran and DAPI to exclude non-viable cells, then FACS analyzed for Alexa 488 signal.

FIG. 3.13 shows that Over-expression of Atg12 rescues Plac8 KD inhibition of tumor formation, but is individually tumor inhibitory. (A,B) The vector control, 3× Flag-tagged Atg12 over-expression, Plac8 shRNA knock down, and plac8 knock-down with 3× Flag-tagged Atg12 over-expressing cells were injected into nude mice at 5×105 cells per injection. The mice were measured every week for tumor burden starting after 2 weeks post injection, and ending after 4 weeks for mp53/Ras transformed cells and 5 weeks for CAPAN-2 cells. Each cell line was injected twelve times and significance levels are P<0.001 for Plac8 KD and Atg12 over-expressing cells compared to matched vector controls and Plac8 KD with Atg12 over-expression cells. (C,D) The vector control, 3× Flag-tagged Atg12 over-expression, Plac8 shRNA knock down, and Plac8 knock-down with 3× Flag-tagged Atg12 over-expressing protein lysates were immunoblotted for p62, LC3, 3× Flag-tag, Plac8 and beta-Tubulin.

FIG. 3.14 shows that Over-expression of Atg12 rescues Plac8 KD inhibition of autophagosomal/lysosomal fusion. (A,B) GFP-LC3 expressing vector, Plac8 shRNA KD, and Plac8 shRNA KD with 3× Flag-tagged Atg12 over-expressioning mp53/Ras transformed and CAPAN-2 cells were nutrient starved in HBSS for 15 minutes, fixed and immunofluorescently stained for Lamp2. Cells were imaged via confocal microscopy. Images were analyzed with ImageJ to highlight and quantify colocalization. (C,D) Colocalization is inhibited in Plac8 shRNA KD by 82% in mp53/Ras cells and 60% in CAPAN-2 cells. The colocalization inhibition by Plac8 KD is rescued with expression of the shRNA resistant Plac8. n>50 cells for all cell lines; P<0.001 for shPlac8/Vector vs. Vector/Vector or P<0.01 for shPlac8/Vector vs. shPlac8/Atg12 for both mp53/Ras transformed and CAPAN-2 cells.

FIG. 4.1 shows that mp53 and Ras synergistically induce autophagosomal/lysosomal fusion. (A) GFP-LC3 expressing YAMC, mp53, Ras and mp53/Ras transformed cells were nutrients starved in HBSS for 15 minutes, fixed and immunofluorescently stained for Lamp2. Cells were imaged via confocal microscopy. Images were analyzed with ImageJ to highlight and quantify colocalization. (B) Colocalization is inhibited in Plac8 shRNA KD by 82% in mp53/Ras cells and 60% in CAPAN-2 cells. The colocalization inhibition by Plac8 KD is rescued with expression of the shRNA resistant Plac8. n>50 cells for all cell lines; P<0.01 for mp53/Ras transformed cells vs. YAMC, mp53, or Ras expressing cells.

FIG. 4.2 shows that mp53 and Ras synergistically induce autophagosome formation. (A) GFP-LC3 expressing YAMC, mp53, Ras and mp53/Ras transformed cells were grown under normal maintenance conditions, were then fixed in methanol and imaged via confocal microscopy. (B) The ImageJ program Watershed Segmentation was used to quantify the amount of GFP in punctae versus the generalized GFP-LC3 signal. Mp53/Ras transformed cells show a synergistic increase in the amount of GFP-LC3 punctae per GFP-LC3 signal compared to YAMC, mp53, or Ras expressing cells. n>20 cells for all cell lines; P<0.001 for mp53/Ras transformed cells vs. YAMC, mp53, or Ras expressing cells.

FIG. 4.3 shows that mp53 and Ras cooperatively induce p62 degradation and LC3 conversion. YAMC, mp53, Ras and mp53/Ras transformed cells were grown under normal condition, treated with 250 nM of Rapamycin for 24 hrs., or 10 mM of 3-methyladenine (3MA) for 24 hrs. The cells were then harvested and lysed for protein. Protein lysates were immunoblotted for p62, LC3 and protein loading control beta-Tubulin. p62 and LC3 levels are cooperatively suppressed by mp53 and Ras under normal growth condition. Treatment with Rapamycin, which stimulates autophagy by mTOR inactivation, suppresses p62 and LC3 protein levels in YAMC, mp53 and Ras, but not mp53/Ras transformed cells. Accumulation of p62 and LC3 occurs in mp53, Ras and mp53/Ras transformed cells with 3MA treatment.

FIG. 4.4 shows that mp53 and Ras synergistically inactivate mTOR. YAMC, mp53, Ras and mp53/Ras transformed cell lystates were immunoblotted for phosphoThr389-p70S6K, a specific phosophorylation site for activated mTOR, total p70S6K, p62, LC3, Plac8, and protein loading control beta-Tubulin. p70S6K is only de-phosphorylated in the mp53/Ras transformed cells. p62 and LC3 proteins levels are also decreased only in mp53/Ras transformed cells.

FIG. 4.5 shows that Atg12 shRNA-mediated KD and Atg12 over-expression inhibit tumor formation, where as Atg12 shRNA-mediated KD with shRNA resistant Atg12 expression restores tumor formation. (A,B) The vector control, 3× Flag-tagged Atg12 over-expression, Atg12 shRNA knock down, and Atg12 knock-down with 3× Flag-tagged Atg12 over-expressing cells were injected into nude mice at 5×105 cells per injection. The mice were measured every week for tumor burden starting after 2 weeks post injection, and ending after 4 weeks. The Atg12 knock down the 3× Flag-tagged Atg12 overexpressing cells show no tumors by 4 weeks, where as the vector control and Atg12 KD with 3× Flag-tagged Atg12 expression cell lines form tumors. Each cell line was injected twelve times and significance levels are P<0.001 for Atg12 KD and Atg12 over-expressing cells compared to matched vector controls and Atg12 KD with Atg12 over-expression cells.

FIG. 4.6 shows that Atg12 shRNA-mediated KD inhibits autophagy, Atg12 overexpression stimulates autophagy, and Atg12 shRNA-mediated KD with shRNA resistant Atg12 expression restores autophagy to vector control levels. The vector control, 3× Flag-tagged Atg12 over-expression, Atg12 shRNA knockdown, and Atg12 knock-down with 3× Flag-tagged Atg12 over-expressing protein lysates were immunoblotted for p62, LC3, 3× Flag-tag, Atg12 and beta-Tubulin. Atg12 shRNA KD accumulation of the autophagic markers p62, LC3-I, and LC3-II can be rescued by expressing 3× Flag-tagged Atg12 in mp53/Ras transformed and CAPAN-2 cells. p62 and LC3 levels are further depressed by overexpression of Atg12 alone, indicating a further induction in autophagy. Atg12 shRNA-mediated KD with shRNA resistant Atg12 expression restores p62 and LC3 protein levels to vector control levels, indicating that the rate of autophagy is back to vector control levels.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHOD OF TREATING CANCER

In one aspect, the compositions and methods disclosed herein relate to the treatment of cancer. As one of skill in the art can appreciate, the present disclosure provides for the inhibition of tumor formation and proliferation by modulating autophagy rates. Thus disclosed herein are methods of treating a cancer in a subject comprising administering to the subject an agent that modulates the rate of autophagy in the cancer.

“Treatment,” “treat,” or “treating” mean a method of reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. Therefore, in the disclosed methods, “treatment” can refer to a 10%, 20%, 30%, 40%, 50%, 60%, 70%, 80%, 90%, or 100% reduction in the severity of an established disease or the disease progression. For example, a disclosed method for reducing the effects of prostate cancer is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition.

1. Multistep Carcinogenesis

It is generally accepted that cancer is a disease that occurs from the accumulation of multiple mutations that alter the normal cell programming to confer the malignant phenotype. Several key studies in the 1950's led to the understanding that human cancer incidence was on average directly proportional to the fourth to sixth power of elapsed time, which indicated that on average four to six events were required for tumorigenesis. The multi-step hypothesis was further refined by Peto et. al. who demonstrated that tumor incidence resulting from carcinogen exposure in mice was related to the time of carcinogen exposure onset and not to the age of the mice, suggesting that tumorigenesis was to due to the accumulation of multiple events over time and not simply due to age. It was suggested even early on that these events could be somatic genetic mutations, and these events were later shown to be discrete mutations in genes termed oncogenes which were found in a wide variety of cancers. In concordance with the early epidemiological data, these mutations occur in a stepwise fashion, where progressive accumulation of discrete genetic mutations are correlated with progressive clinical stages of pre-malignant to malignant disease in many cancers. In colon cancer the earliest mutations, often of the APC gene, result in hyperplastic colonic epithelial cells that are otherwise normal. Moreover hereditary, somatic mutations of the APC gene increase the incidence and accelerate the development of colon cancer, and accumulation of mutations in c-Ki-Ras, and c-Ha-Ras, deleted in colon cancer (DCC) and p53 genes correlate with the transformation into fully malignant cells.

While correlative epidemiological and histopathological data are consistent with a multi-step model of carcinogenesis, the causal relationship for multiple genetic mutations in malignant transformation was established by experiments demonstrating that normal cells were only transformed upon introduction of at least two oncogenic mutations in murine cells, and three mutations in human cells. Furthermore in vivo transgenic mouse experiments support the notion that malignant tumorigenesis requires multiple oncogenic mutations. For example, co-expression of the oncogenes ras and myc in transgenic mice markedly increases tumor initiation compared to mice carrying single oncogenic mutations. These in vitro and in vivo experiments established that multiple oncogenic mutations are required for malignant transformation, however, it was mechanistically unclear why individual oncogenes were insufficient for transformation while cooperative oncogenes conferred the cancer phenotype.

2. Oncogene Cooperation

The cancer phenotype conferred by cooperating oncogenes is characterized by specific biological properties including but not limited to, infinite replicative potential, resistance to apoptosis, insensitivity to anti-growth signals, independence from growth promoting signals, invasion into surrounding tissue, and angiogenesis for the metabolic needs of the growing tumor. These acquired properties can arise from the contribution of individual oncogenes, but could also emerge from the synergistic interaction or cooperation of oncogenic mutations. For example, in primary rat Schwann cells, oncogenic Raf activation increases p21 expression leading to inhibition of cyclincdk activity resulting in cell cycle arrest. However, co-expression of SV40 large T antigen or dominant negative p53 abrogates p21 induction and stimulates cellular proliferation. These data indicate that Raf activation and loss of p53 function interact to regulate cyclin-cdk activity thereby conferring uncontrolled proliferation on malignant cells. Similarly, Fanidi et al. demonstrated that while fibroblast cells expressing c-myc alone exhibited both increased proliferation and apoptosis, co-introduction of Bcl-2 inhibited apoptosis giving rise to hyper proliferative cells. Therefore, the hyperproliferative nature of these cells emerges from the interaction of c-myc and Bcl-2. In a mouse model of acute myelogenous leukemia (AML), which is characterized by rapidly proliferating, undifferentiated cells, neither the constitutively activated tyrosine kinase, BCR-ABL nor the leukemic fusion protein AML1 alone inhibit myeloid differentiation. When expressed together, however, myeloid differentiation is inhibited, resulting in AML, indicating that the malignant loss of differentiation emerges only when both oncogenic mutations are present.

More recently it was demonstrated that mouse colonic epithelial cells acquired alignant migration and invasion by co-expression of oncogenic H-Ras^{V12} (Ras) H175 and dominant p53 negative (mp53) rather than by individual expression of either oncogene indicating that the properties emerge from oncogene cooperation. Mechanistic studies revealed that Ras simultaneously induced two parallel pathways that both activated and inhibited RhoA activity, a gene important for migration and invasion, resulting in no net change in RhoA activation. Simultaneous introduction of mp53 relieved the inhibition of RhoA activity resulting in a net activation of RhoA and associated migration and invasion. The cooperative effect of mp53 and Ras in invasion depend on both RhoA and downstream up-regulation of matrix metalloprotease-9 (MMP-9) mRNA and protein, a gene implicated in early tumor growth and angiogenesis. Perturbation of the synergistic up-regulation of MMP-9 via short hairpin RNA (shRNA) expression back to normal cell expression levels inhibited mp53/Ras cell invasion and tumor formation in nude mice. These data indicated that multiple acquired cancer cell properties are controlled by effectors modulated synergistically downstream of cooperating oncogenic mutations.

Cooperative regulation of gene expression, such as synergistic up-regulation of MMP-9 by Ras and mp53, represents a class of cooperative alterations that are essential for the cancer phenotype. The synergistic up-regulation of MMP-9 by Ras and mp53 was not be predicted from analysis of either oncogene alone, thus providing a rational for expanding the candidate approach that discovered MMP-9 into a genomic analysis with the aim to identify all genes regulated synergistically by Ras activation and p53 loss-of-function. This analysis identified 95 genes, termed “cooperation response genes” (CRGs) (Tables 1 and 2). Perturbation of 14 out of 24 (50%) tested CRGs significantly reduced tumor formation while perturbation of 1 out of 14 (7%) tested non-CRGs, genes not synergistically regulated by mp53 and Ras, affected tumor formation. These data indicated that the CRGs are enriched for genes essential to the cancer phenotype suggesting that this approach is efficient in identifying new cancer therapeutic targets. Out of the cooperation response genes currently known to contribute to the cancer phenotype Plac8 had the largest inhibitory effect on tumor formation upon perturbation, and is required for human colorectal adenocarcinoma tumorigenicity, but was of unknown function in cancer. These data demonstrated that Plac8 is an essential gene to the cancer phenotype in the presence of Ras, p53 and other oncogenic mutations in various cell backgrounds. This strict requirement for plac8 expression for the cancer phenotype prompted in depth investigation into Plac8 function in cancer.

TABLE 1
Cooperation Response Genes

| | | | | Expression | Synergy | Expression | Synergy |
|---|---|---|---|---|---|---|---|
| | | | | mp53/Ras | Score, | mp53/Ras | Score, |
| | | | | vs. YAMC, | Raw | vs. YAMC, | Norm |
| GO Biological | | | | Raw Data | Data, | Norm Data | Data, |
| Process | Gene Symbol | GenBank ID | Affymetrix ID | (fold) | p < 0.01 | (fold) | p < 0.01 |
| Signal | Arhgap24 | BC025502 | 1424842_a_at | 0.08 | 0.29 | 0.07 | 0.31 |
| Transduction | Centd3 | AI851258 | 1419833_s_at | 3.64 | 0.87 | 3.39 | 0.83 |
| | Dgka | BC006713 | 1418578_at | 0.30 | 0.79 | 0.28 | 0.88 |
| | Dixdc1 | BB758432 | 1435207_at | 0.38 | 0.85 | 0.36 | 0.93 |
| | Dusp15 | AF357887 | 1426189_at | 0.57 | 0.84 | 0.51 | 0.89 |
| | Ephb2 | AV221401 | 1425016_at | 0.15 | 0.58 | 0.14 | 0.62 |
| | F2rl1 | NM_007974 | 1448931_at | 2.15 | 0.93** | 2.07 | 0.82 |
| | Fgf18 | NM_008005 | 1449545_at | 0.38 | 0.89 | 0.37 | 0.99# |
| | Fgf7 | NM_008008 | 1422243_at | 7.43 | 0.93** | 7.08 | 0.85 |
| | Garnl3 | BB131106 | 1433553_at | 0.28 | 0.88 | 0.27 | 0.93 |
| | Gpr149 | BB126999 | 1438210_at | 4.09 | 0.55 | 3.87 | 0.53 |
| | Hbegf | L07264 | 1418350_at | 4.57 | 0.99# | 4.44 | 0.90** |
| | Igfbp2 | AK011784 | 1454159_a_at | 0.15 | 0.37* | 0.15 | 0.43* |
| | Jag2 | AV264681 | 1426431_at | 0.24 | 0.86 | 0.23 | 0.91 |
| | Ms4a10 | AK008019 | 1432453_a_at | 0.24 | 0.73 | 0.24 | 0.82 |
| | Pard6g | NM_053117 | 1420851_at | 0.35 | 0.79 | 0.33 | 0.90 |
| | Plxdc2 | BB559706 | 1418912_at | 0.03 | 0.36 | 0.03 | 0.41 |
| | Prkcm | AV297026 | 1447623_s_at | 0.24 | 0.90* | 0.23 | 1.03# |
| | Prkg1 | BB516668 | 1444232_at | 0.23 | 0.86* | 0.23 | 0.95* |
| | Rab40b | AV364488 | 1436566_at | 0.32 | 0.85* | 0.31 | 0.93* |
| | Rasl11a | AK004371 | 1429444_at | 0.42 | 0.87 | 0.41 | 0.95 |
| | Rb1 | NM_009029 | 1417850_at | 0.28 | 0.74 | 0.27 | 0.83 |
| | Rgs2 | AF215668 | 1419248_at | 3.91 | 0.66 | 3.70 | 0.62 |
| | Rprm | NM_023396 | 1422552_at | 0.29 | 0.69 | 0.30 | 0.81 |
| | Sbk1 | BC025837 | 1451190_a_at | 0.40 | 0.81 | 0.41 | 0.91 |
| | Sema3d | BB499147 | 1429459_at | 0.17 | 0.72* | 0.16 | 0.80* |
| | Sema7a | AA144045 | 1459903_at | 4.77 | 0.68 | 4.41 | 0.61 |
| | Sfrp2 | NM_009144 | 1448201_at | 0.13 | 0.27 | 0.13 | 0.31 |
| | Stmn4 | NM_019675 | 1418105_at | 0.36 | 0.73 | 0.34 | 0.78 |
| | Wnt9a | AV273409 | 1436978_at | 0.37 | 0.89 | 0.35 | 1.00# |
| Metabolism/ | Abat | BF462185 | 1433855_at | 0.20 | 0.90* | 0.20 | 0.94# |
| Transport | Abca1 | BB144704 | 1421840_at | 0.14 | 0.59 | 0.13 | 0.65 |
| | Ank | NM_020332 | 1450627_at | 21.76 | 0.64 | 20.34 | 0.62 |
| | Atp8a1 | AW610650 | 1454728_s_at | 0.20 | 0.90* | 0.19 | 0.96# |
| | Chst1 | NM_023850 | 1449147_at | 7.98 | 0.74 | 7.61 | 0.70 |
| | Cpz | AF356844 | 1426251_at | 0.18 | 0.76 | 0.17 | 0.83 |
| | Eno3 | NM_007933 | 1417951_at | 5.46 | 0.77 | 4.69 | 0.75 |
| | Kctd15 | BB091366 | 1435339_at | 6.41 | 0.82 | 6.01 | 0.70 |
| | Ldhb | AV219418 | 1434499_a_at | 0.17 | 0.56 | 0.17 | 0.62 |
| | Man2b1 | BC005430 | 1416340_a_at | 0.31 | 0.83 | 0.29 | 0.91 |
| | Mtus1 | BB699957 | 1454824_s_at | 0.23 | 0.85** | 0.22 | 0.94* |
| | Nbea | AA986379 | 1452251_at | 0.24 | 0.81 | 0.23 | 0.90 |
| | Pla2g7 | AK005158 | 1430700_a_at | 11.07 | 0.55 | 10.67 | 0.50 |
| | Pltp | NM_011125 | 1417963_at | 0.33 | 0.88 | 0.30 | 0.98# |
| | Scn3b | BE951842 | 1435767_at | 0.08 | 0.59 | 0.07 | 0.57 |
| | Slc14a1 | AW556396 | 1428114_at | 9.25 | 0.42 | 9.20 | 0.39 |
| | Slc27a3 | BB147793 | 1427180_at | 0.32 | 0.81 | 0.31 | 0.89 |
| | Sms | NM_009214 | 1421052_a_at | 4.00 | 0.97# | 3.84 | 0.89 |
| | Sod3 | NM_011435 | 1417633_at | 3.98 | 0.96# | 4.03 | 0.90** |
| Cell | Ccl9 | AF128196 | 1417936_at | 8.07 | 0.92 | 7.90 | 0.82 |
| Adhesion | Col9a3 | BG074456 | 1460693_a_at | 0.25 | 0.39 | 0.25 | 0.43 |
| | Cxcl1 | NM_008176 | 1419209_at | 9.83 | 1.02# | 9.71 | 0.84 |
| | Cxcl15 | NM_011339 | 1421404_at | 16.13 | 0.83* | 15.43 | 0.70 |
| | Espn | NM_019585 | 1423005_a_at | 0.23 | 0.67 | 0.23 | 0.76 |
| | Eva1 | BC015076 | 1448265_x_at | 0.25 | 0.86* | 0.24 | 0.96# |
| | Fhod3 | BG066491 | 1435551_at | 0.19 | 0.61** | 0.17 | 0.67** |
| | Igsf4a | NM_018770 | 1417378_at | 18.17 | 0.71 | 16.89 | 0.70 |
| | Mcam | NM_023061 | 1416357_a_at | 0.15 | 0.63 | 0.15 | 0.70 |
| | Mmp15 | NM_008609 | 1422597_at | 0.31 | 0.83 | 0.30 | 0.90 |
| | Parvb | BI134721 | 1438672_at | 4.77 | 0.92** | 4.48 | 0.86 |
| | Pvrl4 | BC024948 | 1451690_a_at | 0.39 | 0.88 | 0.36 | 0.97# |
| Transcriptional | Ankrd1 | AK009959 | 1420992_at | 3.78 | 0.51 | 3.88 | 0.46 |
| Regulators | Hey2 | NM_013904 | 1418106_at | 0.20 | 0.73 | 0.20 | 0.79 |
| | Hmga1 | NM_016660 | 1416184_s_at | 12.21 | 0.83 | 11.38 | 0.82 |
| | Hmga2 | X58380 | 1450781_at | 14.96 | 0.90** | 14.88 | 0.87 |
| | Hoxc13 | AF193796 | 1425874_at | 0.42 | 0.83 | 0.43 | 0.97 |
| | Id2 | BF019883 | 1435176_a_at | 0.24 | 0.61 | 0.25 | 0.69 |
| | Id4 | BB121406 | 1423259_at | 0.10 | 0.39 | 0.09 | 0.41 |
| | Lass4 | BB006809 | 1417782_at | 0.27 | 0.69 | 0.25 | 0.72 |
| | Notch3 | NM_008716 | 1421965_s_at | 0.18 | 0.62 | 0.17 | 0.70 |
| | Pitx2 | U80011 | 1424797_a_at | 0.38 | 0.77 | 0.35 | 0.83 |
| | Satb1 | AV172776 | 1416007_at | 0.23 | 0.80* | 0.22 | 0.87* |
| Apoptosis | Dapk1 | BC021490 | 1427358_a_at | 0.17 | 0.58 | 0.16 | 0.62 |
| | Dffb | AV300013 | 1437051_at | 0.35 | 0.86 | 0.35 | 0.95 |
| | Fas | NM_007987 | 1460251_at | 0.35 | 0.83 | 0.35 | 0.96 |
| | Noxa | NM_021451 | 1418203_at | 0.05 | 0.26 | 0.05 | 0.27 |
| | Perp | NM_022032 | 1416271_at | 0.17 | 0.70 | 0.17 | 0.75 |
| Unknown | Bbs7 | BG074932 | 1454684_at | 0.50 | 0.89 | 0.50 | 1.01# |
| Function | Ckmt1 | NM_009897 | 1417089_a_at | 0.43 | 0.89 | 0.40 | 0.93* |
| | Elavl2 | BB105998 | 1421883_at | 0.40 | 0.72* | 0.39 | 0.83* |
| | Gca | BC021450 | 1451451_at | 0.34 | 0.85* | 0.33 | 0.95* |
| | Mpp7 | AK012883 | 1455179_at | 0.13 | 0.44 | 0.13 | 0.46 |
| | Mrpl15 | AV306676 | 1430798_x_at | 3.18 | 0.98# | 3.08 | 0.88 |
| | Oaf | BC025514 | 1424086_at | 5.01 | 0.99# | 5.08 | 0.90 |
| | Plac8 | AF263458 | 1451335_at | 3.40 | 0.89 | 3.21 | 0.88 |
| | Rai2 | BB770528 | 1452358_at | 0.26 | 0.80 | 0.25 | 0.85 |
| | Sbsn | AI507307 | 1459898_at | 0.41 | 0.72 | 0.38 | 0.78 |
| | Serpinb2 | NM_011111 | 1419082_at | 9.07 | 0.92# | 8.91 | 0.90* |
| | Tex15 | NM_031374 | 1420719_at | 0.16 | 0.59 | 0.15 | 0.59 |
| | Tnfrsf18 | AF229434 | 1422303_a_at | 0.20 | 0.56 | 0.20 | 0.65 |
| | Unc45b | AV220213 | 1436939_at | 0.22 | 0.83 | 0.21 | 0.82 |
| | Zfp385 | NM_013866 | 1418865_at | 0.36 | 0.85 | 0.37 | 0.98# |
| Other | Bex1 | NM_009052 | 1448595_a_at | 0.14 | 0.38* | 0.14 | 0.45* |
| | Daf1 | BE686894 | 1443906_at | 0.11 | 0.41 | 0.11 | 0.43 |
| | Tnnt2 | L47552 | 1424967_x_at | 9.42 | 0.87 | 10.11 | 0.80 |

Unnamed Cooperation Response Genes

| | | | Up/Down |
|---|---|---|---|
| Gene Symbol | GenBank ID | Affymetrix ID | Regulated |
| — | BB333822 | 1446179_at | Up |
| — | BB016042 | 1443437_at | Up |
| — | AV254043 | 1439944_at | Up |
| 2010204K13Rik | NM_023450 | 1421498_a_at | Up |
| 2310002L13Rik | AK009098 | 1453275_at | Up |
| 2610528A11Rik | BF580962 | 1435639_at | Up |
| A130040M12Rik | C85657 | 1428909_at | Up |
| AI467606 | BB234337 | 1433465_a_at | Up |
| AI467606 | BB234337 | 1433466_at | Up |
| B630019K06Rik | BB179847 | 1433452_at | Up |
| Prl2c2///Prl2c3/// | X75557 | 1427760_s_at | Up |
| Prl2c4 | | | |
| — | AA266723 | 1448021_at | Down |
| — | AV133559 | 1459971_at | Down |
| — | BB767109 | 1439734_at | Down |
| — | BB133117 | 1441636_at | Down |
| — | AW543723 | 1441971_at | Down |
| — | BB353853 | 1438310_at | Down |
| — | BM118398 | 1435981_at | Down |
| — | BG076276 | 1445758_at | Down |
| — | BB306828 | 1455298_at | Down |
| — | BQ266693 | 1442073_at | Down |
| — | AV254764 | 1456951_at | Down |
| 1700007K13Rik | AK005731 | 1428705_at | Down |
| 2210023G05Rik | BC027185 | 1424968_at | Down |
| 2310038E17Rik | AK009671 | 1432976_at | Down |
| 2410066E13Rik | BB167663 | 1434581_at | Down |
| 6230424C14Rik | BE949277 | 1441972_at | Down |
| 8030476L19Rik | BB068813 | 1454354_at | Down |
| 9930013L23Rik | AK018112 | 1429987_at | Down |
| A930008G19Rik | BM248711 | 1455428_at | Down |
| A930037G23Rik | BE957307 | 1454628_at | Down |
| BC013672 | BC013672 | 1451777_at | Down |
| BC037703 | AV231983 | 1455241_at | Down |
| C030027H14Rik | BB358264 | 1442175_at | Down |
| C130026I21Rik/// | BC007193 | 1425078_x_at | Down |
| LOC100041885 | | | |
| C130092O11Rik | BG071013 | 1437306_at | Down |
| D330028D13Rik | BB478071 | 1434428_at | Down |
| Dzip1/// | AI509011 | 1452792_at | Down |
| LOC100045776 | | | |
| Dzip1/// | AI509011 | 1428469_a_at | Down |
| LOC100045776 | | | |
| LOC100044927/// | NM_009398 | 1418424_at | Down |
| Tnfaip6 | | | |
| LOC100045546 | BB121406 | 1450928_at | Down |
| LOC100047292 | BI905111 | 1434889_at | Down |
| Acad11 | BQ031255 | 1433545_s_at | Down |
| Acad11 | BQ031255 | 1454647_at | Down |
| Adamts20 | AI450842 | 1456901_at | Down |
| AI956758 | AV234963 | 1460003_at | Down |
| Abi3bp | BC026627 | 1427054_s_at | Down |
| Adcy1 | AI848263 | 1456487_at | Down |
| Apol2 | BB312717 | 1441054_at | Down |
| Dmxl2 | AK018275 | 1428749_at | Down |
| Depdc7 | BC013499 | 1424303_at | Down |
| Ceecam1 | AV323203 | 1435345_at | Down |
| Brunol5 | BB381558 | 1434969_at | Down |
| Glis3 | BB207363 | 1430353_at | Down |
| Grhl3 | AV231424 | 1436932_at | Down |
| Gria3 | BM220576 | 1434728_at | Down |
| Limch1 | AV024662 | 1435106_at | Down |
| Limch1 | BM117827 | 1435321_at | Down |
| Mreg | AV298358 | 1437250_at | Down |
| Ms4a2 | AV241486 | 1443264_at | Down |
| Npr3 | BG066982 | 1435184_at | Down |
| Plekha7 | BF159528 | 1455343_at | Down |
| Ptpdc1 | AV254040 | 1433823_at | Down |
| Slain1 | BB704967 | 1424824_at | Down |
| Slc7a2 | AV244175 | 1436555_at | Down |
| Svop | AK003981 | 1452663_at | Down |

A synergy score smaller than 1 indicates a synergistic or non-additive change in gene expression in response to multiple as compared to single oncogenic mutations. The p-values estimate the level of confidence that the synergy score is less than one. Synergy scores and associated p-values were calculated as described in Methods. For all synergy scores, p-values are p < 0.01, except as indicated (**, p < 0.05; *, p < 0.1; #, not significantly less than 1).

CRGs encode proteins involved in the regulation of cell signaling, transcription, apoptosis, metabolism, transport or adhesion (Table 1), and in large proportion appear misexpressed in human cancer. For 47 out of the 75 CRGs tested co-regulation was found in primary human colon cancer and our murine colon cancer cell model. Moreover three of theses genes (EphB2, HB-EGF and Rb) also have been shown to play a causative role in tumor formation. In addition, altered expression of 29 CRGs has been found in a variety of human cancers (Table 1).

The relevance of differentially expressed genes for malignant cell transformation was assessed by genetic perturbation of a series of 24 CRGs (excluding those with an established role in tumor formation, EphB2, HB-EGF and Rb) and 14 genes responding to p53175H and/or activated H-Ras12V in a non-cooperative manner (non-CRGs). Perturbed genes were chosen across a broad range of biological functions, levels of differential expression and synergy scores. These perturbations were carried out in mp53/Ras cells with the goal to reestablish expression of the manipulated genes at levels relatively close to those found in YAMC control cells, and to monitor subsequent tumor formation following sub-cutaneous injection of these cells into immuno-compromised mice. Of the perturbed genes 18 were up- and 20 down-regulated in mp53/Ras cells, relative to YAMC (Table 2).

Tumor volume was measured weekly for 4 weeks following injection into nude mice of murine and human cancer cells. Reversal of the changes in CRG expression significantly reduced tumor formation by mp53/Ras cells in 14 out of 24 cases (Table 2), indicating a critical role in malignant transformation for a surprisingly large fraction of these genes. Perturbation of Plac8, Jag2 and HoxC13 gene expression had the strongest effects. In addition, perturbation of two CRGs, Fas and Rprm, that alone produced significant yet milder changes in tumor formation were combined. This yielded significantly increased efficacy in tumor inhibition as compared with the respective single perturbations. Thus, even genetic perturbations of CRGs that seem to have relatively smaller effects when examined on their own show evidence of being essential when analyzed in combination.

TABLE 2
Tumor formation by mp53/Ras cells following perturbation of individual cooperation response genes
(CRGs)

| | | | | | % Change in | | |
|---|---|---|---|---|---|---|---|
| | | | Expression | | Tumor Volume | p Value | |
| Gene | Gene | Synergy | mp53/Ras vs. | Number of | (Perturbed vs. | (Wilcox | p Value |
| Name | Function | Score | YAMC (fold) | Injections (n) | Control) | n) | (t-test) |
| | | | | | Smaller | | |
| Plac8 | Unknown | 0.88 | 3.21 | 9 | −100 | 0.0006 | 0.0001 |
| Jag2 | Signaling | 0.86 | 0.24 | 8 | −94 | 0.0003 | 0.0007 |
| HoxC13 | Transcription | 0.83 | 0.42 | 8 | −76 | 0.005 | 0.002 |
| Sod3 | Metabolism | 0.90** | 4.03 | 16 | −72 | 0.004 | 0.001 |
| Gpr149 | Signaling | 0.53 | 3.87 | 12 | −70 | 0.006 | 0.05 |
| Dffb | Apoptosis | 0.86 | 0.35 | 8 | −69 | 0.005 | 0.01 |
| Fgf7 | Signaling | 0.85 | 7.08 | 6 | −68 | 0.004 | 0.01 |
| Rgs2 | Signaling | 0.62 | 3.70 | 18 | −60 | 0.0002 | 0.006 |
| Perp | Apoptosis | 0.70 | 0.17 | 16 | −59 | 0.0008 | 0.002 |
| Zfp385 | Unknown | 0.85 | 0.36 | 8 | −59 | 0.007 | 0.005 |
| Wnt9a | Signaling | 0.89 | 0.37 | 8 | −50 | 0.002 | 0.002 |
| Fas | Apoptosis | 0.83 | 0.35 | 10 | −43 | 0.02 | 0.02 |
| Pla2g7 | Metabolism | 0.50 | 10.67 | 14 | −42 | 0.02 | 0.04 |
| Rprm | Signaling | 0.69 | 0.29 | 12 | −36 | 0.01 | 0.04 |
| | | | | | No Significant | | |
| | | | | | Change | | |
| Hmga2 | Transcription | 0.87 | 14.88 | 10 | −34 | 0.96 | 0.43 |
| Igsf4a | Migration | 0.70 | 16.89 | 10 | −33 | 0.37 | 0.31 |
| Sfrp2 | Signaling | 0.27 | 0.13 | 10 | −25 | 0.23 | 0.24 |
| Id2 | Transcription | 0.61 | 0.24 | 6 | −18 | 0.70 | 0.41 |
| Noxa | Apoptosis | 0.26 | 0.05 | 8 | −18 | 0.30 | 0.33 |
| Sema3d | Signaling | 0.72* | 0.17 | 6 | −16 | 0.67 | 0.40 |
| Hmga1 | Transcription | 0.82 | 11.38 | 14 | −5 | 0.48 | 0.91 |
| Plxdc2 | Signaling | 0.36 | 0.03 | 6 | 24 | 0.13 | 0.08 |
| Id4 | Transcription | 0.39 | 0.10 | 6 | 79 | 0.20 | 0.14 |
| | | | | | Larger | | |
| Slc14a1 | Metabolism | 0.39 | 9.20 | 6 | 180 | 0.008 | 0.002 |

3. Plac8

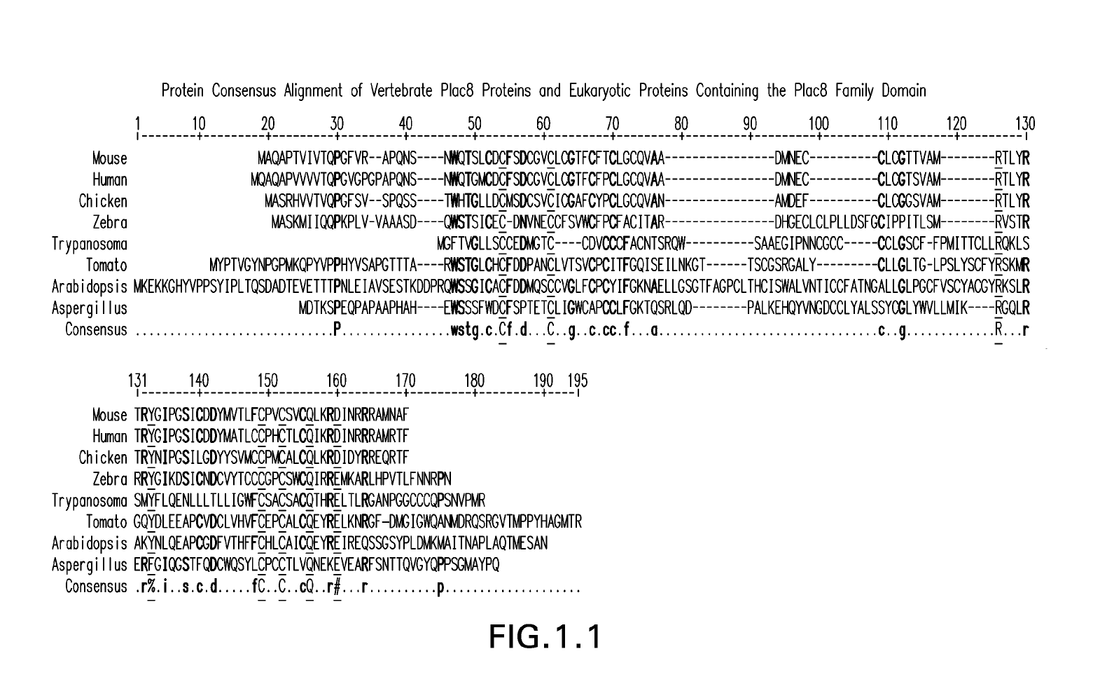

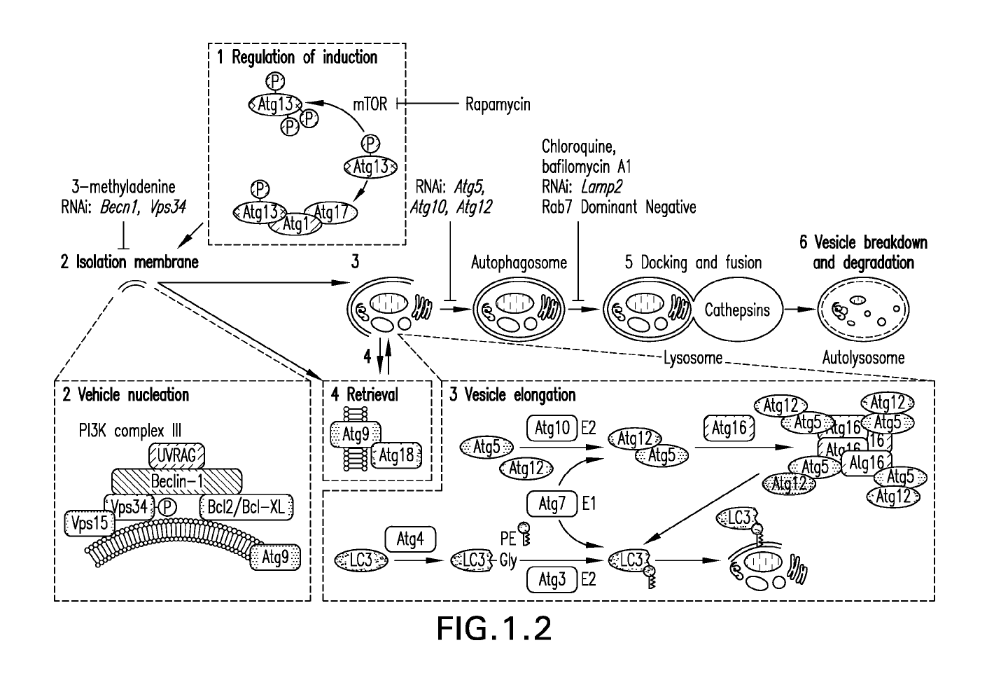

The Plac8 gene can be phylogenetically traced back to Euteleostomi or bony vertebrates, which encodes a 112 amino acid protein in mouse and a 115 amino acid protein in humans. The Plac8 protein also contains a more ancient, conserved eukaryotic protein domain designated Plac8 Family Domain, which dominates 80% of the Plac8 protein and is highly enriched in cysteine residues (FIG. 1.1). In Arabidopsis these proteins containing the Plac8 Family Domain are up-regulated in response to oxidative stress and overexpression of Plac8 Domain Containing Arabidopsis proteins in Arabidopsis and yeast confers resistance to heavy metal toxicity, suggesting a role in resistance to cellular stressors. In mammalian cells over-expression experiments in Rat1a fibroblasts suggested that Plac8 interacts with and activates Akt and Mdm2 resulting in p53 degradation and, as a consequence, suppression of apoptosis in response chemotherapeutic agents. Plac8, however, is required for tumor formation in p53-deficient murine and human malignant cells indicating an alternative function of Plac8 in cancer. An important clue came from Plac8 knock-out mice that are viable but show retarded killing of phagocytosed bacteria in neutrophils derived from these mice. It was also demonstrated by Ledford et al. that the Plac8 protein was enriched in granular fraction of neutrophils, representing modified lysosomes, suggesting a possible sub-cellular localization of the Plac8 protein.

4. Autophagy and Cancer

Autophagy or “self eating” is a biological process in which cells degrade internal components in bulk via lysosomes. The cell utilizes this process for antigen presentation, recycling of amino acids from damaged proteins, degradation of defunct organelles, and subsequent generation of metabolites for energetic requirements. Macroautophagy (herein referred to as autophagy) was first described in Saccharomyces cerevisiae, where autophagosome formation was observed under conditions of carbon or nitrogen deprivation and 15 genes required for autophagy were identified (ATG1-15). It was later discovered that these ATG genes were highly conserved in higher eukaryotes, including mammals.

Autophagy can be broken down into two phases, autophagosome formation and autophagosme/lysosome fusion (also referred to as autophagosome maturation or degradation) (FIG. 1.2). In the autophagosome formation phase Atg genes are activated by metabolic stress sensing mechanisms, such as AMP-activated protein kinase (AMPK) activation. Conversely Atg genes are deactivated by growth factor pathway activation, most notably Phosphoinositol-3 kinase (PI3-Kinase). The AMPK phosphorylation cascade inactivates mTOR, a protein kinase that is currently understood as the main integration point to modulate autophagy, and the PI3-Kinase phosphorylation cascade activates mTOR. Activation of mTOR leads to the phosphorylation of the autophagy genes Atg1 (ULK) and Atg13 and subsequent inhibition of the autophagy process, so inactivation of mTOR activates autophagy. Activation of the Atg1/Atg13 complex starts a cascade of phosphorylations and ubiquitin-like conjugations of other Atg genes, most notably the conjugation of Atg12-Atg5. The Atg12-Atg5 protein conjugate facilitates autophagosome membrane formation and LC3 (Atg8) conjugation to phosphotydilserine on the interior and exterior membrane surfaces of the autophagosome, which is observed on immunoblotting as conversion of LC3-I (the unconjugated form) to LC3-II (the conjugated form). This allows for the internalization of aggregated damaged proteins and the sequestering protein p62/Sequestrome1, a protein that aggregates misfolded/damaged proteins in the cell. LC3-II tagging also marks the completed endosome as an autophagosome in the cell. Once the autophagosome formation is completed the components inside must be degraded to recycle the metabolites trapped in macromolecular polymers. This is accomplished by fusion of the autophagosome with a lysosome, a process that requires proteins such as Rab7, a ras-like GTPase involved in late endosomal trafficking and endosomal/lysosomal fusion, and Lamp2, an internal lysosomal protein required for endosomal/lysosomal fusion. Degradation of the autophagosome components LC3-II and p62 indicates successful completion of the fusion process. The importance of autophagy as a mechanism for bulk cellular recycling is clear, as it is the only known means for large scale degradation and clearance of organelles and protein aggregates.

In the context of cancer autophagy can be both tumor inhibitory and a survival strategy for cancer. Initially it was discovered that Beclin1 and its binding partner UVRAG, which are genes involved in autophagosome formation, were frequently inactivated in human cancers at single loci. Moreover, Beclin1 was found to act as a haploinsufficient tumor suppressor in mice, suggesting that autophagy was tumor inhibitory. Further correlative evidence for a role of autophagy in tumor suppression is provided by the fact that class I PI3K pathway activating mutations are common in cancer and activate mTOR, a direct inhibitor of the autophagy process. It has also been demonstrated that KO of Atg5 and over-expression of Bcl-2 accelerate tumor formation of immortalized baby mouse kidney cells, once again suggesting that autophagy is tumor suppressive.

Evidence for a requirement of autophagy for tumor formation also involves the previously discussed gene Beclin1. Tumors that Beclin1^{+/-} arose in mice still expressed protein levels of Beclin1 found in tissues of Beclin1^{+/+} animals (Yue, et al., 2003) and, unlike other mouse tumor models involving heterozygote tumor suppressor genes, no Beclin1^{+/-} tumors lost expression of the remaining WT allele suggesting some basal requirement of autophagy. In addition, it was also shown that Beclin1 shRNA mediated gene knock down in human cancer cells inhibited proliferation suggesting that autophagy was still required at some level. More recently it has been demonstrated the mutation or nullification of the tumor suppressor p53, which occurs in >50% of all human cancers, induces autophagy, resulting in an increase in cell survival under nutrient starvation and hypoxia, similar to what neoplastic cells experience in vivo due to insufficient circulatory perfusion. Furthermore inhibition of the autophagy process at autophagosomal/lysosomal fusion by cholorquine or bafilomycin A1 has also been shown to be tumor inhibitory.

The seemingly conflicting data led to the question as to whether autophagy is induced or inhibited in malignant transformation and if so, is de-regulation of the autophagy process required for the transformed phenotype. Herein is disclosed for the first time that autophagy is cooperatively induced by mp53 and Ras and this activation of the autophagy process is required for the transformed phenotype.

In particular, it is disclosed herein that either an increase or decrease in the rate of autophagy in a cell can lead to malignant transformation. Accordingly, the genes, proteins, and enzyme involved in autophagy can increase or decrease depending on the cancer. It is further understood, that a gene, protein, or enzyme that activates autophagy in a malignant transformation can be inhibited to decrease the amount of autophagy to more approximate normal non-malignant tissue. Similarly, a gene, protein, or enzyme that inhibits autophagy can be inhibited to increase the amount of autophagy to more approximate normal non-malignant tissue. Thus, agents for use in the methods disclosed herein can be designed to increase or decrease autophagy depending on the target molecule. In other words, if the target for modulating autophagy is a gene, protein, or enzyme that activates autophagy, then the agent will inhibit the target. If by contrast, the target for modulating autophagy is a gene, protein, or enzyme is an inhibitor of autophagy and is decreased in the malignant cell, then the agent will activate the target to increase expression.

As referred to herein, “decrease” can refer to any change that results in a smaller amount of a symptom, composition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed.

An “increase” can refer to any change that results in a larger amount of a symptom, composition, or activity. Thus, for example, an increase in the amount of Jag2 can include but is not limited to a 10%, 20%, 30%, 40%, 50%, 60%, 70%, 80%, 90%, or 100% increase.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Enhance,” “enhancing,” and “enhancement” mean to increase an activity, response, condition, disease, or other biological parameter. This can include but is not limited to the doubling, tripling, quadrupling, or any other factor of increase in activity, response, condition, or disease. This may also include, for example, a 10% increase in the activity, response, condition, or disease as compared to the native or control level. Thus, the increase can be a 10, 20, 30, 40, 50, 60, 70, 80, 90, 100, 150, 200, 300, 400, 500% or any amount of increase in between as compared to native or control levels.

Targets for the disclosed methods can be any gene, protein, enzyme or other molecule that modulates autophagy. For example, the target of the disclosed methods can be a cooperation response gene that is necessary for activation of autophagy and thus malignant transformation, such as Plac8. Thus disclosed herein in one embodiment are methods wherein the target of the disclosed modulation methods is a cooperation response genes selected for the group consisting of Arhgap24, Centd3, Dgka, Dixdc, Dusp15, EphB2, F2rl1, Fgf18, Fgf7, Garn13, Gpr149, Hbegf, Igfbp2, Jag2, Ms4a10, Pard6g, Plxdc2, Rab40b, Rasl11a, Rb1, Rgs2, Rprm, Sbk1, Sema3d, Sema7a, Sfrp2, Stmn4, Wnt9a, Abat, Abca1, Ank, Atp8a1, Chst1, Cpz, Eno3, Kctd15, Ldhb, Man2b1, Mtus1, Nbea, Pla2g7, Pltp, Prss22, Rspo3, Scn3b, Slc14a1, Slc27a3, Sms, Sod3, Ccl9, Col9a3, Cxcl1, Cxcl15, Espn, Eva1, Fhod3, FHOS2, Igsf4a, Mcam, Mmp15, Parvb, Pvrl4, Ankrd1, Hey2, Hmga1, Hmga2, Hoxc13, Id2, Id4, Lass4, Notch3, Pitx2, Satb1, Dapk1, Dffb, Fas, Notch3, Noxa, Perp, Bbs7, Ckmt1, Elav12, Gca, Mpp7, Mrpplf4, Oaf, Plac8, Rai2, Sbsn, Serpinb2, Tex15, Tnfrsf18, Unc45b, Zfp385, Bex1, Daf1, Tnnt2, Zac1 as well as the cooperation response genes identified by the Genbank accession number AV133559, BM118398, BB353853, BB381558, AV231983, AI848263, AV244175, BF159528, AV231424, AV234963, BC013499, AV254040, BG071013, AK003981, BG066186, AK005731, BC027185, AK009671, AV323203, AI509011, BM220576, BQ173895, AV024662, BB207363, BC026627, AK017369, BQ031255, BC007193, BE949277, AK018275, BB704967, BB312717, AK018112, BI905111, BE957307, BG066982, BB358264, BB478071, AV298358, BB767109, AA266723, AV241486, BB133117, AI450842, and AW543723 or any other cooperative response gene identified in Tables 1 and 2. Also disclosed herein are methods wherein the target of the disclosed modulation methods is selected from the group consisting of ATG1, ATG2, ATG3, ATG4, ATG5, ATG6, ATG7, ATG8, ATG8, ATG10, ATG11, ATG12, ATG13, ATG14, ATG15, ATG16, ATG17, ATG18, ATG19, ATG20, ATG21, ATG22, ATG23, ATG24, ATG25, ATG26, ATG27, ATG28, ATG29, ATG30, ATG31, ATG101, LC3, RAB7, VPS15, VPS35, UVRAG, Beclin1, BCL2, BCL-XL, ULK1 ULK2, ULK3, ULK4, DapK1, FIP200, TSC1, TSC2, AMPK, Redd1, CAMKKbeta, LKB, MO25, STRAD, PTEN, mTOR, Raptor, Deptor, Rictor, Protor, PRAS40, LST8, Rheb, RAG A, RAG B, RAG C, RAG D, AKT, PDK1, PI3K, IRS1, Insulin/IGF1 receptor, ERK, MEK, RAF, SIN1, MAP4K3, Plac8, Dominant negative (DN) Rab7, Rab7, Dominant active (DA) Rab7, Rab40b, SLC7A5, and SLC3A2.

Because the disclosed modulator targets can be natural inhibitors or enhancers of autophagy, specifically disclosed herein is the use of agents that can reverse this activity of the target thereby returning the rate of autophagy towards the normal state. For example, disclosed herein, the agents can be an antibody, siRNA, small molecule inhibitory drug, shRNA, or peptide mimetic that specifically binds to a gene that modulates the rate of autophagy. For example, the agent can be a siRNA that inhibits the expression of Plac8 such as shRNA's shPlac8 155, 240 and 461 siRNA constructs. Because Plac8 is an activator of autophagy, the knock-down of Plac8 expression inhibits autophagy activation. Examples of molecules that activate autophagy include but are not limited to Plac8, ATG1, ATG2, ATG3, ATG4, ATG5, ATG6, ATG7, ATG8, ATG8, ATG10, ATG11, ATG12, ATG13, ATG14, ATG15, ATG16, ATG17, ATG18, ATG19, ATG20, ATG21, ATG22, ATG23, ATG24, ATG25, ATG26, ATG27, ATG28, ATG29, ATG30, ATG31, ATG101, LC3, RAB7, VPS15, VPS35, UVRAG, Beclin1, BCL2, BCL-XL, ULK1 ULK2, ULK3, ULK4, DapK1, FIP200, TSC1, TSC2, DA Rab7, AMPK, Redd1, CAMKKbeta, LKB, MO25, STRAD, and PTEN. By contrast, some agents can inhibit a molecule whose expression inhibits autophagy thus the knock-down or blocking of such a molecule would activate autophagy. Examples, of molecules that inhibit autophagy include but are not limited to mTOR, Raptor, Deptor, Rictor, Protor, PRAS40, LST8, Rheb, RAG A, RAG B, RAG C, RAG D, AKT, PDK1, PI3K, IRS1, Insulin/IGF1 receptor, DN Rab7, ERK, MEK, RAF, SIN1, MAP4K3, SLC7A5, and SLC3A2.

Alternatively, the use of a potential target molecule as an agent is also contemplated herein, wherein the expression of the agent molecule drives autophagy to a rate that is inhibitory to the cancer either by increasing an already activated state, further inhibiting inhibited states, inhibiting an activated state, or activating an inhibited state. It is further contemplated herein that one method of modulating the rate of autophagy is through the expression or over-expression of agents such as nucleic acids, peptides, proteins, and enzymes that do not act on a target, but modulate autophagy by competing with the target (in the case of competitive inhibition) or alter autophagy by acting in their normal manner. For example, the use of a dominant negative gene that results in a necessary autophagy activating or inhibiting protein having decreased or absent expression. The overexpression of a molecule such as, for example ATG5 or ATG12, whose overexpression inhibits autophagy. Alternatively, an autophagy activator such as Plac8 could be used to activate autophagy when the malignant transformation slows the rate of autophagy.

Because the tumor formation is dependant on the rate of autophagy, proteins such as ATG12 which promote autophagy when expressed at normal levels become tumor inhibitory when overexpressed. Thus, contemplated herein are methods of treating a cancer comprising administering an agent that modulates autophagy; wherein the agent is a natural regulator of autophagy, and the administration of the agent causes the expression or overexpression of the modulator; and wherein over-expression of the expression modulator is tumor inhibitory, such as, for example the overexpression of ATG12 or expression of DA Rab7.

It is understood and herein contemplated that the activity of the cooperation response gene can be modulated by modulating the expression of one or more, two or more, three or more, four or more, or five or more of the CRG. It is further understood and herein contemplated that the expression can be inhibited or enhanced. It is understood and herein contemplated that those of skill in the art will understand whether to inhibit or enhance the activity of one or more cooperation response genes. For example, one of skill in the art will understand that where the expression of a particular CRG is up-regulated in a cancer, one of skill in the art will want to administer an agent that decreases or inhibits the up-regulation of the CRG. Similarly, where the expression of a particular CRG is down-regulated in a cancer, one of skill in the art will want to administer an agent that increases or enhances the expression of the down-regulated CRG. Moreover, it is contemplated herein that one method of treating cancer is to administer an agent that targets down-regulated CRG's in combination with an agent that targets up-regulated CRG's. Therefore, for example, disclosed herein are methods of treating cancer comprising administering to the subject one or more agents that inhibits the activity of one or more cooperation response genes. Also disclosed are methods wherein the cooperation response gene is selected from the group consisting of Plac8, Sod3, Gpr149, Fgf7, Cxcl1, Rgs2, Pla2g7, Igsf4a, and Hmga1. Also disclosed are methods of treating cancer comprising administering to the subject one or more agents that enhances the activity of one or more cooperation response genes. Also disclosed are methods wherein the cooperation response gene is selected from the group consisting of Jag2, HoxC13, Dffb, Dapk1, Daf1, EphB2, Rab40b, Notch3, Dgka, Zac1, Perp, Zfp385, Wnt9a, Fas, Rprm, Sfrp2, Id2, Noxa, Sema3d, Plxdc2, Id4, and Slc14a1. Thus, for example, disclosed herein are method of treating a cancer comprising administering to a subject one or more agents such as (+)-chelidonine, 0179445-0000, 0198306-0000, 1,4-chrysenequinone, 15-delta prostaglandin J2, 2,6-dimethylpiperidine, 4-hydroxyphenazone, 5186223, 6-azathymine, acenocoumarol, alpha-estradiol, altizide, alverine, alvespimycin, amikacin, aminohippuric acid, amoxicillin, amprolium, ampyrone, antimycin A, arachidonyltrifluoromethane, atractylo side, azathioprine, azlocillin, bacampicillin, baclofen, bambuterol, beclometasone, benzylpenicillin, betaxolol, betulinic acid, biperiden, boldine, bromocriptine, bufexamac, buspirone, butacaine, butirosin, calycanthine, canadine, canavanine, carbarsone, carbenoxolone, carbimazole, carcinine, carmustine, cefalotin, cefepime, ceftazidime, cephaeline, chenodeoxycholic acid, chlorhexidine, chlorogenic acid, chlorpromazine, chlortalidone, cinchonidine, cinchonine, clemizole, co-dergocrine mesilate, CP-320650-01, CP-690334-01, dacarbazine, demeclocycline, dexibuprofen, dextromethorphan, dicycloverine, diethylstilbestrol, diflorasone, diflunisal, dihydroergotamine, diloxanide, dinoprostone, diphemanil metilsulfate, diphenylpyraline, doxylamine, droperidol, epirizole, epitiostanol, esculetin, estradiol, estropipate, ethionamide, etofenamate, etomidate, eucatropine, famotidine, famprofazone, fendiline, fisetin, fludrocortisone, flufenamic acid, flupentixol, fluphenazine, fluticasone, fluvastatin, fosfosal, fulvestrant, gabexate, galantamine, gemfibrozil, genistein, glibenclamide, gliquidone, glycocholic acid, gossypol, gramine, guanadrel, halcinonide, haloperidol, harpagoside, hexamethonium bromide, homochlorcyclizine, hydroxyzine, idoxuridine, ifosfamide, indapamide, iobenguane, iopanoic acid, iopromide, isoetarine, isoxsuprine, isradipine, ketorolac, ketotifen, lanatoside C, lansoprazole, laudanosine, letrozole, levodopa, levomepromazine, lidocaine, liothyronine, lisinopril, lisuride, LY-294002, lynestrenol, meclofenamic acid, meclofenoxate, medrysone, mefloquine, mepacrine, methapyrilene, methazolamide, methyldopa, methylergometrine, metoclopramide, mevalolactone, mometasone, monensin, monorden, naftopidil, nalbuphine, naltrexone, napelline, naphazoline, naringin, niclosamide, niflumic acid, nimesulide, nomifensine, noretynodrel, norfloxacin, orphenadrine, oxolinic acid, oxprenolol, papaverine, pentolonium, pepstatin, perphenazine, PF-00562151-00, phenelzine, phenindione, pheniramine, phthalylsulfathiazole, pinacidil, pioglitazone, piperine, piretanide, piribedil, pirlindole, PNU-0230031, pralidoxime, pramocaine, praziquantel, prednisone, Prestwick-1100, Prestwick-981, probenecid, prochlorperazine, proglumide, propofol, protriptyline, racecadotril, riboflavin, rifabutin, rimexolone, roxithromycin, santonin, SB-203580, SC-560, scopoletin, scriptaid, seneciphylline, sirolimus, sitosterol, sodium phenylbutyrate, solanine, spectinomycin, spiradoline, SR-95531, SR-95639A, sulfadimidine, sulfaguanidine, sulfanilamide, sulfathiazole, tanespimycin, terbutaline, terguride, thalidomide, thiamazole, thiamphenicol, thioridazine, ticarcillin, ticlopidine, tinidazole, tiratricol, tolfenamic acid, tremorine, trichostatin A, trifluoperazine, troglitazone, tyloxapol, ursodeoxycholic acid, valproic acid, vanoxerine, vidarabine, vincamine, vorinostat, wortmannin, yohimbic acid, yohimbine, or zidovudine.

Also disclosed herein is the use of small molecules such as Chloroquine and Bafilomycin A1 to modulate autophagy.

It is understood and contemplated herein that one means of treating cancer is through the administration of a single agent that modulates the expression or activity of one or more, two or more, three or more, four or more, or five or more cooperative response genes. It is further understood that it one or more agents that modulate the expression or activity of one or more cooperative response genes can be administered. For example, it is contemplated herein that one method of treating a cancer is to administer an agent that It is understood and herein contemplated that modulation of expression is not the only means for modulating the activity of one or more cooperation response genes and such means can be accomplished by any manner known to those of skill in the art. Therefore, for example, disclosed herein are methods of treating cancer wherein the activity of the cooperation response gene is inhibited by the administration of an antibody, siRNA, small molecule inhibitory drug, shRNA, or peptide mimetic that is specific for the protein encoded by the cooperation response gene. Also disclosed are methods wherein the antibody, siRNA, small molecule inhibitory drug, or peptide mimetic is specific for the protein encoded by Plac8, Sod3, Gpr149, Fgf7, Rgs2, Pla2g7, Igsf4a, or Hmga1.

In another aspect, the disclosed methods of treating cancer can be combined with anti-cancer agents such as, for example, chemotherapeutics or anti-oxidants known in the art. Therefore, disclosed herein are methods of treating a cancer in a subject comprising administering to the subject one or more anti-cancer agents and one or more agents that modulate the activity of one or more cooperation response genes. Further disclosed are methods wherein the anti-cancer agent is a chemotherapeutic or antioxidant compound. Also disclosed are methods wherein the anti-cancer agent is a histone deacetylase inhibitor.

It is understood that the disclosed compositions and methods can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, colorectal adenocarcinoma, pancreatic adenocarcinoma, leukemias, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, gastric cancer, colon cancer, cervical cancer, cervical carcinoma, breast cancer, and epithelial cancer, bone cancers, renal cancer, bladder cancer, genitourinary cancer, esophageal carcinoma, large bowel cancer, metastatic cancers hematopoietic cancers, sarcomas, Ewing's sarcoma, synovial cancer, soft tissue cancers; and testicular cancer. Thus disclosed herein are methods of treating wherein the cancer is selected form the group of cancers consisting of lymphoma, B cell lymphoma, T cell lymphoma, mycosis fungoides, Hodgkin's Disease, leukemias, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, gastric cancer, colon cancer, colorectal adenocarcinoma, pancreatic adenocarcinoma, cervical cancer, cervical carcinoma, breast cancer, and epithelial cancer, bone cancers, renal cancer, bladder cancer, genitourinary cancer, esophageal carcinoma, large bowel cancer, metastatic cancers hematopoietic cancers, sarcomas, Ewing's sarcoma, synovial cancer, soft tissue cancers; and testicular cancer.

Compounds and methods disclosed herein may also be used for the treatment of precancer conditions such as cervical and anal dysplasias, other dysplasias, severe dysplasias, hyperplasias, atypical hyperplasias, and neoplasias.

Thus, for example disclosed herein are methods of treating pancreatic or colo-rectal cancer in a subject, comprising administering to the subject an agent that modulates autophagy in a cancer cell.

The disclosed agents can take the form of any molecule that can be used in the disclosed methods such as a nucleic acid, morphilinos, shRNAs, siRNAs, peptides, proteins, enzymes, antibodies, small molecules, peptide mimetics, dominant negative mutants, dominant active mutants, and natural inhibitor or activator of autophagy.

### C. METHODS FOR SCREENING FOR AGENTS THAT TREAT CANCER

It is understood and herein contemplated that a single agent may not be effective in the treatment of a cancer or the modulation of one or more of the targets identified by the methods disclosed herein. Moreover, the modulation through a given route may have toxic effects on the subject. Therefore, there is a need to screen for additional agents that modulate autophagy thereby inhibiting tumor formation or proliferation. Thus, disclosed herein are methods of screening for an agent that treats cancer comprising measuring the rate of autophagy in a cancer cell and a non-cancerous control cell, determining of the rate of autophagy in the cancer cell is increased or decreased relative to the rate of autophagy in the control cell, contacting a cancer cell with the agent, and measuring the rate of autophagy, wherein an agent that modulates the rate of autophagy in the cancer cell in a direction towards the rate of autophagy in the control cell indicates an agent that can treat cancer.

It is further understood that, as noted above, the agents identified herein can act on specific autophagy associate modulators referred to herein as “targets.” Said targets in the disclosed methods can be cooperation response genes selected from the list of cooperation response genes consisting of Arhgap24, Centd3, Dgka, Dixdc, Dusp15, Ephb2, F2rl1, Fgf18, Fgf7, Garn13, Gpr149, Hbegf, Igfbp2, Jag2, Ms4a10, Pard6g, Plxdc2, Rab40b, Rasl11a, Rb1, Rgs2, Rprm, Sbk1, Sema3d, Sema7a, Sfrp2, Stmn4, Wnt9a, Abat, Abca1, Ank, Atp8a1, Chst1, Cpz, Eno3, Kctd15, Ldhb, Man2b1, Mtus1, Nbea, Pla2g7, Pltp, Prss22, Rspo3, Scn3b, Slc14a1, Slc27a3, Sms, Sod3, Ccl9, Col9a3, Cxcl1, Cxcl15, Espn, Eva1, Fhod3, FHOS2, Igsf4a, Mcam, Mmp15, Parvb, Pvrl4, Ankrd1, Hey2, Hmga1, Hmga2, Hoxc13, Id2, Id4, Lass4, Notch3, Pitx2, Satb1, Dapk1, Dffb, Fas, Noxa, Perp, Bbs7, Ckmt1, Elav12, Gca, Mpp7, Mrpplf4, Oaf, Plac8, Rai2, Sbsn, Serpinb2, Tex15, Tnfrsf18, Unc45b, Zfp385, Bex1, Daf1, Tnnt2, Zac1 and the cooperation response genes identified by the Genbank accession numbers AV133559, BM118398, BB353853, BB381558, AV231983, AI848263, AV244175, BF159528, AV231424, AV234963, BC013499, AV254040, BG071013, AK003981, BG066186, AK005731, BC027185, AK009671, AV323203, AI509011, BM220576, BQ173895, AV024662, BB207363, BC026627, AK017369, BQ031255, BC007193, BE949277, AK018275, BB704967, BB312717, AK018112, BI905111, BE957307, BG066982, BB358264, BB478071, AV298358, BB767109, AA266723, AV241486, BB133117, AI450842, and AW543723. It is a further embodiment that the target can be a known modulator of autophagy selected from the group consisting of Arhgap24, Centd3, Dgka, Dixdc, Dusp15, Ephb2, F2rl1, Fgf18, Fgf7, Garn13, Gpr149, Hbegf, Igfbp2, Jag2, Ms4a10, Pard6g, Plxdc2, Rab40b, Rasl11a, Rb1, Rgs2, Rprm, Sbk1, Sema3d, Sema7a, Sfrp2, Stmn4, Wnt9a, Abat, Abca1, Ank, Atp8a1, Chst1, Cpz, Eno3, Kctd15, Ldhb, Man2b1, Mtus1, Nbea, Pla2g7, Pltp, Prss22, Rspo3, Scn3b, Slc14a1, Slc27a3, Sms, Sod3, Ccl9, Col9a3, Cxcl1, Cxcl15, Espn, Eva1, Fhod3, FHOS2, Igsf4a, Mcam, Mmp15, Parvb, Pvrl4, Ankrd1, Hey2, Hmga1, Hmga2, Hoxc13, Id2, Id4, Lass4, Notch3, Pitx2, Satb1, Dapk1, Dffb, Fas, Noxa, Perp, Bbs7, Ckmt1, Elav12, Gca, Mpp7, Mrpplf4, Oaf, Plac8, Rai2, Sbsn, Serpinb2, Tex15, Tnfrsf18, Unc45b, Zfp385, Bex1, Daf1, Tnnt2, Zac1 and the cooperation response genes identified by the Genbank accession numbers AV133559, BM118398, BB353853, BB381558, AV231983, AI848263, AV244175, BF159528, AV231424, AV234963, BC013499, AV254040, BG071013, AK003981, BG066186, AK005731, BC027185, AK009671, AV323203, AI509011, BM220576, BQ173895, AV024662, BB207363, BC026627, AK017369, BQ031255, BC007193, BE949277, AK018275, BB704967, BB312717, AK018112, BI905111, BE957307, BG066982, BB358264, BB478071, AV298358, BB767109, AA266723, AV241486, BB133117, AI450842, AW543723, ATG1, ATG2, ATG3, ATG4, ATG5, ATG6, ATG7, ATG8, ATG8, ATG10, ATG11, ATG12, ATG13, ATG14, ATG15, ATG16, ATG17, ATG18, ATG19, ATG20, ATG21, ATG22, ATG23, ATG24, ATG25, ATG26, ATG27, ATG28, ATG29, ATG30, ATG31, ATG101, LC3, RAB7, VPS15, VPS35, UVRAG, Beclin1, BCL2, BCL-XL, ULK1 ULK2, ULK3, ULK4, DapK1, FIP200, TSC1, TSC2, AMPK, Redd1, CAMKKbeta, LKB, MO25, STRAD, PTEN, mTOR, Raptor, Deptor, Rictor, Protor, PRAS40, LST8, Rheb, RAG A, RAG B, RAG C, RAG D, AKT, PDK1, PI3K, IRS1, Insulin/IGF1 receptor, ERK, MEK, RAF, SIN1, MAP4K3, Dominant negative (DN) Rab7, Rab7, Dominant active (DA) Rab7, Rab40b, SLC7A5, and SLC3A2. Alternatively, the agent being screened can be a cooperative response gene or modulator of autophagy selected from the group consisting of ATG1, ATG2, ATG3, ATG4, ATG5, ATG6, ATG7, ATG8, ATG8, ATG10, ATG11, ATG12, ATG13, ATG14, ATG15, ATG16, ATG17, ATG18, ATG19, ATG20, ATG21, ATG22, ATG23, ATG24, ATG25, ATG26, ATG27, ATG28, ATG29, ATG30, ATG31, ATG101, LC3, RAB7, VPS15, VPS35, UVRAG, Beclin1, BCL2, BCL-XL, ULK1 ULK2, ULK3, ULK4, DapK1, FIP200, TSC1, TSC2, AMPK, Redd1, CAMKKbeta, LKB, MO25, STRAD, PTEN, mTOR, Raptor, Plac8, Deptor, Rictor, Protor, PRAS40, LST8, Rheb, RAG A, RAG B, RAG C, RAG D, AKT, PDK1, PI3K, IRS1, Insulin/IGF1 receptor, ERK, MEK, RAF, SIN1, MAP4K3, Dominant negative (DN) Rab7, Rab7, Dominant active (DA) Rab7, Rab40b, SLC7A5, and SLC3A2.

It is understood and herein contemplated that the desired effect of the agent on the cooperation response gene depends on the activity of the cooperation response gene and its effect on autophagy. In some cases for inhibition of a cancer to occur, the cooperation response gene must be inhibited and in other cases enhanced. In still other cases the modulator of autophagy must be activated and even other cases autophagy must be inhibited. Thus, it is understood and herein contemplated that disclosed agents can modulate the activity of the target through inhibition or enhancement. Therefore, disclosed herein are methods for screening for an agent that treats cancer comprising contacting the agent decreases the rate of autophagy. Also disclosed are methods wherein the agent increases the rate of autophagy. In particular, disclosed herein are methods for screening for an agent that treats cancer comprising contacting the agent with the one or more targets, wherein the agent inhibits the activity of the target in a manner such that tumor proliferation is inhibited, wherein the target is a cooperation response gene.

Also disclosed herein are methods for screening for an agent that treats cancer comprising contacting the agent with the one or more targets, wherein the agent modulates the activity of the target in a manner such that tumor proliferation is inhibited, wherein the agent modulation of the activity of the target is enhanced. In particular, disclosed herein are methods for screening for an agent that treats cancer comprising contacting the agent with the one or more targets, wherein the agent enhances the activity of the target in a manner such that tumor proliferation is inhibited, wherein the target is a cooperation response gene. Further disclosed are methods wherein the cooperation response gene selected from the group consisting of Jag2, HoxC13, Dffb, Dapk1, Daf1, EphB2, Rab40b, Notch3, Dgka, Zac1, Perp, Zfp385, Wnt9a, Fas, Rprm, Sfrp2, Id2, Noxa, Sema3d, Plxdc2, Id4, and Slc14a1.

### D. METHODS OF IDENTIFYING TARGETS FOR THE TREATMENT OF CANCER

Despite recognition of the multifaceted cellular phenotype of cancers and the need for targeted intervention strategies, identification of such targets, however, is notoriously difficult and unpredictable using techniques known in the art. Therefore, disclosed herein are methods for identifying targets for the treatment of a cancer comprising performing an assay that measures differential expression of a gene or protein and identifying those genes, proteins, or micro RNAs that respond synergistically to the combination of two or more cancer genes.

As used herein, “cancer gene” can refer to any gene that has an effect on the formation, maintenance, proliferation, death, or survival of a cancer. It is understood and herein contemplated that “cancer gene” can comprise oncogenes, tumor suppressor genes, as well as gain or loss of function mutants there of. It is further understood and herein contemplated that where a particular combination of two or more cancer genes is discussed, disclosed herein are each and every permutation of the combination including the use of the gain or loss of functions mutants of the particular genes in the combination. It is further understood and herein contemplated that the disclosed combinations can include an oncogene and a tumor suppressor gene, two oncogenes, two tumor suppressor genes, or any variation thereof where gain or loss of function mutants are used. Thus, for example, disclosed herein are any combination of two or more of the cancer genes selected from the group consisting of ABL1, ABL2, AF15Q14, AF1Q, AF3p21, AF5q31, AKT, AKT2, ALK, ALO17, AML1, AP1, APC, ARHGEF, ARHH, ARNT, ASPSCR1, ATIC, ATM, AXL, BCL10, BCL11A, BCL11B, BCL2, BCL3, BCL5, BCL6, BCL7A, BCL9, BCR, BHD, BIRC3, BLM, BMPR1A, BRCA1, BRCA2, BRD4, BTG1, CBFA2T1, CBFA2T3, CBFB, CBL, CCND1, c-fos, CDH1, c-jun, CDK4, c-kit, CDKN2A-p14ARF, CDKN2A-p161NK4A, CDX2, CEBPA, CEP1, CHEK2, CHIC2, CHN1, CLTC, c-met, c-myc, COL1A1, COPEB, COX6C, CREBBP, c-ret, CTNNB1, CYLD, D10S170, DDB2, DDIT3, DDX10, DEK, EGFR, EIF4A2, ELKS, ELL, EP300, EPS15, erbB, ERBB2, ERCC2, ERCC3, ERCC4, ERCC5, ERG, ETV1, ETV4, ETV6, EVI1, EWSR1, EXT1, EXT2, FACL6, FANCA, FANCC, FANCD2, FANCE, FANCF, FANCG, FEV, FGFR1, FGFR1OP, FGFR2, FGFR3, FH, FIP1L1, FLI1, FLT3, FLT4, FMS, FNBP1, FOXO1A, FOXO3A, FPS, FSTL3, FUS, GAS7, GATA1, GIP, GMPS, GNAS, GOLGA5, GPC3, GPHN, GRAF, HEI10, HER3, HIP1, HIST1H4I, HLF, HMGA2, HOXA11, HOXA13, HOXA9, HOXC13, HOXD11, HOXD13, HRAS, HRPT2, HSPCA, HSPCB, hTERT, IGH, IGK, IGL, IL21R, IRF4, IRTA1, JAK2, KIT, KRAS2, LAF4, LASP1, LCK, LCP1, LCX, LHFP, LMO1, LMO2, LPP, LYL1, MADH4, MALT1, MAML2, MAP2K4, MDM2, MECT1, MEN1, MET, MHC2TA, MLF1, MLH1, MLL, MLLT1, MLLT10, MLLT2, MLLT3, MLLT4, MLLT6, MLLT7, MLM, MN1, MSF, MSH2, MSH6, MSN, MTS1, MUTYH, MYC, MYCL1, MYCN, MYH11, MYH9, MYST4, NACA, NBS1, NCOA2, NCOA4, NF1, NF2, NOTCH1, NPM1, NR4A3, NRAS, NSD1, NTRK1, NTRK3, NUMA1, NUP214, NUP98, NUT, OLIG2, p53, p27, p57, p16, p21, p73, PAX3, PAX5, PAX7, PAX8, PBX1, PCM1, PDGFB, PDGFRA, PDGFRB, PICALM, PIM1, PML, PMS1, PMS2, PMX1, PNUTL1, POU2AF1, PPARG, PRAD-1, PRCC, PRKAR1A, PRO1073, PSIP2, PTCH, PTEN, PTPN11, RAB5EP, RAD51L1, RAF, RAP1GDS1, RARA, RAS, Rb, RB1, RECQL4, REL, RET, RPL22, RUNX1, RUNXBP2, SBDS, SDHB, SDHC, SDHD, SEPT6, SET, SFPQ, SH3GL1, SIS, SMAD2, SMAD3, SMAD4, SMARCB1, SMO, SRC, SS18, SS18L1, SSH3BP1, SSX1, SSX2, SSX4, Stathmin, STK11, STL, SUFU, TAF15, TAL1, TAL2, TCF1, TCF12, TCF3, TCL1A, TEC, TCF12, TFE3, TFEB, TFG, TFPT, TFRC, TIF1, TLX1, TLX3, TNFRSF6, TOP1, TP53, TPM3, TPM4, TPR, TRA, TRB, TRD, TRIM33, TRIP11, TRK, TSC1, TSC2, TSHR, VHL, WAS, WHSC1L1 8, WRN, WT1, XPA, XPC, ZNF145, ZNF198, ZNF278, ZNF384, and ZNFN1A1. It is further understood that the disclosed combinations of two or more cancer genes can comprise 2, 3, 4, 5, 6, 7, 8, 9, or 10 cancer genes.

As discussed above, disclosed herein are combinations of cancer genes, wherein the cancer genes comprise an oncogene and loss of function of a tumor suppressor gene. It is understood and herein contemplated that there are many oncogenes known in the art. Thus, for example, disclosed herein are cancer gene combinations comprising an oncogene and a tumor suppressor gene wherein the oncogene is selected from the list of oncogenes consisting of ras, raf, Bcl-2, Akt, S is, src, Notch, Stathmin, mdm2, abl, hTERT, c-fos, c-jun, c-myc, erbB, HER2/Neu, HER3, c-kit, c-met, c-ret, flt3, AP1, AML1, axl, alk, fms, fps, gip, lck, MLM, PRAD-1, and trk. Therefore, disclosed herein are methods for identifying targets for the treatment of a cancer comprising performing an assay that measures differential expression of a gene, protein or micro RNAs and identifying those genes, proteins or micro RNAs that respond synergistically to the combination of two or more cancer genes, wherein the combination of two or more cancer genes comprises an oncogene and a tumor suppressor gene wherein the oncogene is selected from the list of oncogenes consisting of ras, raf, Bcl-2, Akt, S is, src, Notch, Stathmin, mdm2, abl, hTERT, c-fos, c-jun, c-myc, erbB, HER2/Neu, HER3, c-kit, c-met, c-ret, flt3, AP1, AML1, axl, alk, fms, fps, gip, lck, MLM, PRAD-1, and trk. It is understood that there are other means known in the art to accomplish this task other than evaluating synergistic response of gene expression to a combination of cancer genes. One such method, for example, involves developing rank-order by synergy score, multiplicativity score, or maximum p-value by N-test. While the multiplicativity score and differential expression via the N-test identify somewhat different sets of genes than the additive synergy score, all three methods perform similarly at isolating genes critical to tumor formation from non-essential genes. Thus, disclosed herein are methods for identifying targets for the treatment of a cancer comprising performing an assay that measures differential expression of a gene, protein or micro RNAs, evaluating the expression via additive synergy score, multiplicative synergy score, or N-test, and identifying those genes, proteins or micro RNAs that have differential expression in response to the combination of two or more cancer genes relative to the absence of said cancer genes or the presence of one cancer gene, wherein the combination of two or more cancer genes comprises an oncogene and a tumor suppressor gene wherein the oncogene is selected from the list of oncogenes consisting of ras, raf, Bcl-2, Akt, S is, src, Notch, Stathmin, mdm2, abl, hTERT, c-fos, c-jun, c-myc, erbB, HER2/Neu, HER3, c-kit, c-met, c-ret, flt3, AP1, AML1, axl, alk, fms, fps, gip, lck, MLM, PRAD-1, and trk.

Further disclosed are cancer gene combinations comprising an oncogene and a tumor suppressor gene and/or their gain or loss of function mutants wherein the tumor suppressor gene is selected from the list of tumor suppressor genes consisting of p53, Rb, PTEN, BRCA-1, BRCA-2, APC, p57, p27, p16, p21, p′73, p14ARF, Chek2, NF1, NF2, VHL, WRN, WT1, MEN1, MTS1, SMAD2, SMAD3, and SMAD4. Therefore, disclosed herein are methods for identifying targets for the treatment of a cancer comprising performing an assay that measures differential expression of a gene or protein and identifying those genes, proteins, or micro RNAs that respond synergistically to the combination of two or more cancer genes, wherein the combination of two or more cancer genes comprises an oncogene and a tumor suppressor gene and/or their gain or loss of function mutants wherein the tumor suppressor gene is selected from the list of tumor suppressor genes consisting of p53, Rb, PTEN, BRCA-1, BRCA-2, APC, p57, p27, p16, p21, p′73, p14ARF, Chek2, NF1, NF2, VHL, WRN, WT1, MEN1, MTS1, SMAD2, SMAD3, and SMAD4. Therefore disclosed herein are methods for identifying targets for the treatment of a cancer comprising performing an assay that measures differential expression of a gene or protein and identifying those genes, proteins, or micro RNAs that respond synergistically to the combination of two or more cancer genes, wherein the combination of two or more cancer genes comprises an oncogene and a tumor suppressor gene wherein the oncogene is selected from the list of oncogenes consisting of ras, raf, Bcl-2, Akt, S is, src, Notch, Stathmin, mdm2, abl, hTERT, c-fos, c-jun, c-myc, erbB, HER2/Neu, HER3, c-kit, c-met, c-ret, flt3, AP1, AML1, axl, alk, fms, fps, gip, lck, MLM, PRAD-1, and trk and wherein the tumor suppressor gene is selected from the list of tumor suppressor genes consisting of p53, Rb, PTEN, BRCA-1, BRCA-2, APC, p57, p27, p16, p21, p73, p14ARF, Chek2, NF1, NF2, VHL, WRN, WT1, MEN1, MTS1, SMAD2, SMAD3, and SMAD4. Thus, for example, specifically disclosed are cancer gene combinations comprising p53 and Ras.

It is understood that the cancer gene combinations can include combinations of only oncogenes and/or their gain or loss of function mutants. Therefore, disclosed herein are methods for identifying targets for the treatment of a cancer comprising performing an assay that measures differential expression of a gene or protein and identifying those genes, proteins, or micro RNAs that respond synergistically to the combination of two or more cancer genes, wherein the combination of two or more cancer genes comprises two or more oncogenes wherein the oncogenes are selected from the list of oncogenes consisting of ras, raf, Bcl-2, Akt, S is, src, Notch, Stathmin, mdm2, abl, hTERT, c-fos, c-jun, c-myc, erbB, HER2/Neu, HER3, c-kit, c-met, c-ret, flt3, AP1, AML1, axl, alk, fms, fps, gip, lck, MLM, PRAD-1, and trk. Likewise, it is understood that the cancer gene combinations can include combinations of only tumor suppressor genes and/or their gain or loss of function mutants. Therefore, disclosed herein are methods for identifying targets for the treatment of a cancer comprising performing an assay that measures differential expression of a gene or protein and identifying those genes, proteins, or micro RNAs that respond synergistically to the combination of two or more cancer genes, wherein the combination of two or more cancer genes comprises two or more tumor suppressor genes wherein the tumor suppressor gene is selected from the list of tumor suppressor genes consisting of p53, Rb, PTEN, BRCA-1, BRCA-2, APC, p57, p27, p16, p21, p′73, p14ARF, Chek2, NF1, NF2, VHL, WRN, WT1, MEN1, MTS1, SMAD2, SMAD3, and SMAD4.

The methods disclosed herein can be assayed by any means to measure differential expression of a gene or protein known in the art. Specifically contemplated herein are methods of identifying targets for the treatment of a cancer comprising performing an assay that measures differential expression of a gene. Specifically contemplated are methods of identifying targets for the treatment of a cancer comprising performing an assay that measures differential gene expression, wherein the assay is selected from the group of assays consisting of, Northern analysis, RNAse protection assay, PCR, QPCR, genome microarray, low density PCR array, oligo array, SAGE and high throughput sequencing. Also disclosed herein are methods of identifying targets for the treatment of a cancer comprising performing an assay that measures differential expression of a protein. Specifically contemplated are methods of identifying targets for the treatment of a cancer comprising performing an assay that measures differential protein expression wherein the assay is selected from the group of assays consisting of protein microarray, antibody-based or protein activity-based detection assays and mass spectrometry.

It is understood and herein contemplated that the methods disclosed herein can be combined with additional methods known in the art to further identify the targets, assess the effect of the targets on a cancer or screen for agents that interact with the targets and through the interaction modulate cancer. Therefore, disclosed herein are methods of identifying targets for the treatment of a cancer comprising performing an assay that measures differential expression of a gene or protein and identifying those genes, proteins, or micro RNAs that respond synergistically to the combination of two or more cancer genes and further comprising measuring the effect of the targets on neoplastic cell transformation in vitro, in vitro cell death, in vitro survival, in vivo cell death, in vivo survival, in vitro angiogenesis, in vivo tumor angiogenesis, tumor formation, tumor maintenance, or tumor proliferation. It is also understood that there are many means known in the art for measuring the effect of the targets. One such method is through the perturbation of one or more targets and assaying for a change in the tumor or cancer cells relative to a control. Thus, for example, disclosed herein are methods, wherein the effect of the targets is measured through the perturbation of one or more targets and assaying for a change in the tumor or cancer cells relative to a control wherein a difference in the tumor or cancer cells relative to a control indicates a target that affects the tumor.

### E. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular cancer gene or cooperation response gene is disclosed and discussed and a number of modifications that can be made to a number of molecules including the cancer gene or cooperation response gene are discussed, specifically contemplated is each and every combination and permutation of cancer gene or cooperation response gene and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C—F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Nucleic Acids

There are a variety of molecules disclosed herein that are nucleic acid based, including for example the nucleic acids that encode, for example, Arhgap24, Centd3, Dgka, Dixdc, Dusp15, Ephb2, F2rl1, Fgf18, Fgf7, Garn13, Gpr149, Hbegf, Igfbp2, Jag2, Ms4a10, Pard6g, Plxdc2, Rab40b, Rasl11a, Rb1, Rgs2, Rprm, Sbk1, Sema3d, Sema7a, Sfrp2, Stmn4, Wnt9a, Abat, Abca1, Ank, Atp8a1, Chst1, Cpz, Eno3, Kctd15, Ldhb, Man2b1, Mtus1, Nbea, Pla2g7, Pltp, Prss22, Rspo3, Scn3b, Slc14a1, Slc27a3, Sms, Sod3, Ccl9, Col9a3, Cxcl1, Cxcl15, Espn, Eva1, Fhod3, FHOS2, Igsf4a, Mcam, Mmp15, Parvb, Pvrl4, Ankrd1, Hey2, Hmga1, Hmga2, Hoxc13, Id2, Id4, Lass4, Notch3, Pitx2, Satb1, Dapk1, Dffb, Fas, Noxa, Perp, Bbs7, Ckmt1, Elav12, Gca, Mpp7, Mrpplf4, Oaf, Plac8, Rai2, Sbsn, Serpinb2, Tex15, Tnfrsf18, Unc45b, Zfp385, Bex1, Daf1, Tnnt2, and Zac1 as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556),

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to, for example, Arhgap24, Centd3, Dgka, Dixdc, Dusp15, Ephb2, F2rl1, Fgf18, Fgf7, Garn13, Gpr149, Hbegf, Igfbp2, Jag2, Ms4a10, Pard6g, Plxdc2, Rab40b, Rasl11a, Rb1, Rgs2, Rprm, Sbk1, Sema3d, Sema7a, Sfrp2, Stmn4, Wnt9a, Abat, Abca1, Ank, Atp8a1, Chst1, Cpz, Eno3, Kctd15, Ldhb, Man2b1, Mtus1, Nbea, Pla2g7, Pltp, Prss22, Rspo3, Scn3b, Slc14a1, Slc27a3, Sms, Sod3, Ccl9, Col9a3, Cxcl1, Cxcl15, Espn, Eva1, Fhod3, FHOS2, Igsf4a, Mcam, Mmp15, Parvb, Pvrl4, Ankrd1, Hey2, Hmga1, Hmga2, Hoxc13, Id2, Id4, Lass4, Notch3, Pitx2, Satb1, Dapk1, Dffb, Fas, Noxa, Perp, Bbs7, Ckmt1, Elav12, Gca, Mpp7, Mrpplf4, Oaf, Plac8, Rai2, Sbsn, Serpinb2, Tex15, Tnfrsf18, Unc45b, Zfp385, Bex1, Daf1, Tnnt2, and Zac1 as well as any other protein disclosed herein that are disclosed on Genbank, and these sequences and others are herein incorporated by reference in their entireties as well as for individual subsequences contained therein.

A variety of sequences are provided herein and these and others can be found in Genbank. Those of skill in the art understand how to resolve sequence discrepancies and differences and to adjust the compositions and methods relating to a particular sequence to other related sequences. Primers and/or probes can be designed for any sequence given the information disclosed herein and known in the art.

c) Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

d) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, shRNAs, siRNAs, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of Arhgap24, Centd3, Dgka, Dixdc, Dusp15, Ephb2, F2rl1, Fgf18, Fgf7, Garn13, Gpr149, Hbegf, Igfbp2, Jag2, Ms4a10, Pard6g, Plxdc2, Rab40b, Rasl11a, Rb1, Rgs2, Rprm, Sbk1, Sema3d, Sema7a, Sfrp2, Stmn4, Wnt9a, Abat, Abca1, Ank, Atp8a1, Chst1, Cpz, Eno3, Kctd15, Ldhb, Man2b1, Mtus1, Nbea, Pla2g7, Pltp, Prss22, Rspo3, Scn3b, Slc14a1, Slc27a3, Sms, Sod3, Ccl9, Col9a3, Cxcl1, Cxcl15, Espn, Eva1, Fhod3, FHOS2, Igsf4a, Mcam, Mmp15, Parvb, Pvrl4, Ankrd1, Hey2, Hmga1, Hmga2, Hoxc13, Id2, Id4, Lass4, Notch3, Pitx2, Satb1, Dapk1, Dffb, Fas, Noxa, Perp, Bbs7, Ckmt1, Elav12, Gca, Mpp7, Mrpplf4, Oaf, Plac8, Rai2, Sbsn, Serpinb2, Tex15, Tnfrsf18, Unc45b, Zfp385, Bex1, Daf1, Tnnt2, and Zac1 or the genomic DNA of Arhgap24, Centd3, Dgka, Dixdc, Dusp15, Ephb2, F2rl1, Fgf18, Fgf7, Garn13, Gpr149, Hbegf, Igfbp2, Jag2, Ms4a10, Pard6g, Plxdc2, Rab40b, Rasl11a, Rb1, Rgs2, Rprm, Sbk1, Sema3d, Sema7a, Sfrp2, Stmn4, Wnt9a, Abat, Abca1, Ank, Atp8a1, Chst1, Cpz, Eno3, Kctd15, Ldhb, Man2b1, Mtus1, Nbea, Pla2g7, Pltp, Prss22, Rspo3, Scn3b, Slc14a1, Slc27a3, Sms, Sod3, Ccl9, Col9a3, Cxcl1, Cxcl15, Espn, Eva1, Fhod3, FHOS2, Igsf4a, Mcam, Mmp15, Parvb, Pvrl4, Ankrd1, Hey2, Hmga1, Hmga2, Hoxc13, Id2, Id4, Lass4, Notch3, Pitx2, Satb1, Dapk1, Dffb, Fas, Noxa, Perp, Bbs7, Ckmt1, Elav12, Gca, Mpp7, Mrpplf4, Oaf, Plac8, Rai2, Sbsn, Serpinb2, Tex15, Tnfrsf18, Unc45b, Zfp385, Bex1, Daf1, Tnnt2, and Zac1 or they can interact with the polypeptide. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (kd) less than or equal to 10-6, 10-8, 10-10, or 10-12. A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with kds from the target molecule of less than 10-12 M. It is preferred that the aptamers bind the target molecule with a kd less than 10-6, 10-8, 10-10, or 10-12. Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a kd with the target molecule at least 10, 100, 1000, 10,000, or 100,000 fold lower than the kd with a background binding molecule. It is preferred when doing the comparison for a polypeptide for example, that the background molecule be a different polypeptide. For example, when determining the specificity of Arhgap24, Centd3, Dgka, Dixdc, Dusp15, Ephb2, F2rl1, Fgf18, Fgf7, Garn13, Gpr149, Hbegf, Igfbp2, Jag2, Ms4a10, Pard6g, Plxdc2, Rab40b, Rasl11a, Rb1, Rgs2, Rprm, Sbk1, Sema3d, Sema7a, Sfrp2, Stmn4, Wnt9a, Abat, Abca1, Ank, Atp8a1, Chst1, Cpz, Eno3, Kctd15, Ldhb, Man2b1, Mtus1, Nbea, Pla2g7, Pltp, Prss22, Rspo3, Scn3b, Slc14a1, Slc27a3, Sms, Sod3, Ccl9, Col9a3, Cxcl1, Cxcl15, Espn, Eva1, Fhod3, FHOS2, Igsf4a, Mcam, Mmp15, Parvb, Pvrl4, Ankrd1, Hey2, Hmga1, Hmga2, Hoxc13, Id2, Id4, Lass4, Notch3, Pitx2, Satb1, Dapk1, Dffb, Fas, Noxa, Perp, Bbs7, Ckmt1, Elav12, Gca, Mpp7, Mrpplf4, Oaf, Plac8, Rai2, Sbsn, Serpinb2, Tex15, Tnfrsf18, Unc45b, Zfp385, Bex1, Daf1, Tnnt2, and Zac1 aptamers, the background protein could be Arhgap24, Centd3, Dgka, Dixdc, Dusp15, Ephb2, F2rl1, Fgf18, Fgf7, Garn13, Gpr149, Hbegf, Igfbp2, Jag2, Ms4a10, Pard6g, Plxdc2, Rab40b, Rasl11a, Rb1, Rgs2, Rprm, Sbk1, Sema3d, Sema7a, Sfrp2, Stmn4, Wnt9a, Abat, Abca1, Ank, Atp8a1, Chst1, Cpz, Eno3, Kctd15, Ldhb, Man2b1, Mtus1, Nbea, Pla2g7, Pltp, Prss22, Rspo3, Scn3b, Slc14a1, Slc27a3, Sms, Sod3, Ccl9, Col9a3, Cxcl1, Cxcl15, Espn, Eva1, Fhod3, FHOS2, Igsf4a, Mcam, Mmp15, Parvb, Pvrl4, Ankrd1, Hey2, Hmga1, Hmga2, Hoxc13, Id2, Id4, Lass4, Notch3, Pitx2, Satb1, Dapk1, Dffb, Fas, Noxa, Perp, Bbs7, Ckmt1, Elav12, Gca, Mpp7, Mrpplf4, Oaf, Plac8, Rai2, Sbsn, Serpinb2, Tex15, Tnfrsf18, Unc45b, Zfp385, Bex1, Daf1, Tnnt2, and Zac1. Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a kd less than 10-6, 10-8, 10-10, or 10-12. Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J. 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos. 5,168,053, 5,624,824, 5,683,873, 5,728,521, 5,869,248, and 5,877,162.

2. Nucleic Acid Delivery

In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 107 to 109 plaque forming units (pfu) per injection but can be as high as 1012 pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

3. Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

a) Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids, such as Arhgap24, Centd3, Dgka, Dixdc, Dusp15, Ephb2, F2rl1, Fgf18, Fgf7, Garn13, Gpr149, Hbegf, Igfbp2, Jag2, Ms4a10, Pard6g, Plxdc2, Rab40b, Rasl11a, Rb1, Rgs2, Rprm, Sbk1, Sema3d, Sema7a, Sfrp2, Stmn4, Wnt9a, Abat, Abca1, Ank, Atp8a1, Chst1, Cpz, Eno3, Kctd15, Ldhb, Man2b1, Mtus1, Nbea, Pla2g7, Pltp, Prss22, Rspo3, Scn3b, Slc14a1, Slc27a3, Sms, Sod3, Ccl9, Col9a3, Cxcl1, Cxcl15, Espn, Eva1, Fhod3, FHOS2, Igsf4a, Mcam, Mmp15, Parvb, Pvrl4, Ankrd1, Hey2, Hmga1, Hmga2, Hoxc13, Id2, Id4, Lass4, Notch3, Pitx2, Satb1, Dapk1, Dffb, Fas, Noxa, Perp, Bbs7, Ckmt1, Elav12, Gca, Mpp7, Mrpplf4, Oaf, Plac8, Rai2, Sbsn, Serpinb2, Tex15, Tnfrsf18, Unc45b, Zfp385, Bex1, Daf1, Tnnt2, and Zac1 into the cell without degradation and include a promoter yielding expression of the gene in the cells into which it is delivered. In some embodiments the vectors are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### (1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### (2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### (3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### (4) Large payload viral vectors

Molecular genetic experiments with large human herpesviruses have provided a means whereby large heterologous DNA fragments can be cloned, propagated and established in cells permissive for infection with herpesviruses (Sun et al., Nature Genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA>150 kb to specific cells.

EBV recombinants can maintain large pieces of DNA in the infected B-cells as episomal DNA. Individual clones carried human genomic inserts up to 330 kb appeared genetically stable the maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA>220 kb and to infect cells that can stably maintain DNA as episomes.

Other useful systems include, for example, replicating and host-restricted non-replicating vaccinia virus vectors.

b) Non-Nucleic Acid Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed vectors for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci. USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral intergration systems can also be incorporated into nucleic acids which are to be delivered using a non-nucleic acid based system of deliver, such as a liposome, so that the nucleic acid contained in the delivery system can be come integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

c) In Vivo/Ex Vivo

As described above, the compositions can be administered in a pharmaceutically acceptable carrier and can be delivered to the subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

4. Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 by in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

5. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms/disorder are/is effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody, for treating, inhibiting, or preventing a cancer, the efficacy of the therapeutic antibody can be assessed in various ways well known to the skilled practitioner. For instance, one of ordinary skill in the art will understand that a composition, such as an antibody, disclosed herein is efficacious in treating or inhibiting a cancer in a subject by observing that the composition reduces tumor size or prevents a further increase in other indicators of tumor survival or growth including but not limited to neoplastic cell transformation in vitro, in vitro cell death, in vivo cell death, in vitro angiogenesis, in vivo tumor angiogenesis, tumor formation, tumor maintenance, or tumor proliferation or further decrease in in vitro or in vivo survival.

The compositions that inhibit Arhgap24, Centd3, Dgka, Dixdc, Dusp15, Ephb2, F2rl1, Fgf18, Fgf7, Garn13, Gpr149, Hbegf, Igfbp2, Jag2, Ms4a10, Pard6g, Plxdc2, Rab40b, Rasl11a, Rb1, Rgs2, Rprm, Sbk1, Sema3d, Sema7a, Sfrp2, Stmn4, Wnt9a, Abat, Abca1, Ank, Atp8a1, Chst1, Cpz, Eno3, Kctd15, Ldhb, Man2b1, Mtus1, Nbea, Pla2g7, Pltp, Prss22, Rspo3, Scn3b, Slc14a1, Slc27a3, Sms, Sod3, Ccl9, Col9a3, Cxcl1, Cxcl15, Espn, Eva1, Fhod3, FHOS2, Igsf4a, Mcam, Mmp15, Parvb, Pvrl4, Ankrd1, Hey2, Hmga1, Hmga2, Hoxc13, Id2, Id4, Lass4, Notch3, Pitx2, Satb1, Dapk1, Dffb, Fas, Noxa, Perp, Bbs7, Ckmt1, Elav12, Gca, Mpp7, Mrpplf4, Oaf, Plac8, Rai2, Sbsn, Serpinb2, Tex15, Tnfrsf18, Unc45b, Zfp385, Bex1, Daf1, Tnnt2, and Zac1 interactions disclosed herein may be administered prophylactically to patients or subjects who are at risk for a cancer.

Other molecules that interact with Arhgap24, Centd3, Dgka, Dixdc, Dusp15, Ephb2, F2rl1, Fgf18, Fgf7, Garn13, Gpr149, Hbegf, Igfbp2, Jag2, Ms4a10, Pard6g, Plxdc2, Rab40b, Rasl11a, Rb1, Rgs2, Rprm, Sbk1, Sema3d, Sema7a, Sfrp2, Stmn4, Wnt9a, Abat, Abca1, Ank, Atp8a1, Chst1, Cpz, Eno3, Kctd15, Ldhb, Man2b1, Mtus1, Nbea, Pla2g7, Pltp, Prss22, Rspo3, Scn3b, Slc14a1, Slc27a3, Sms, Sod3, Ccl9, Col9a3, Cxcl1, Cxcl15, Espn, Eva1, Fhod3, FHOS2, Igsf4a, Mcam, Mmp15, Parvb, Pvrl4, Ankrd1, Hey2, Hmga1, Hmga2, Hoxc13, Id2, Id4, Lass4, Notch3, Pitx2, Satb1, Dapk1, Dffb, Fas, Noxa, Perp, Bbs7, Ckmt1, Elav12, Gca, Mpp7, Mrpplf4, Oaf, Plac8, Rai2, Sbsn, Serpinb2, Tex15, Tnfrsf18, Unc45b, Zfp385, Bex1, Daf1, Tnnt2, and Zac1 which do not have a specific pharmaceutical function, but which may be used for tracking changes within cellular chromosomes or for the delivery of diagnostic tools for example can be delivered in ways similar to those described for the pharmaceutical products.

The disclosed compositions and methods can also be used for example as tools to isolate and test new drug candidates for various cancers including but not limited to lymphoma, B cell lymphoma, T cell lymphoma, mycosis fungoides, Hodgkin's Disease, leukemias, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, gastric cancer, colon cancer, cervical cancer, cervical carcinoma, breast cancer, and epithelial cancer, bone cancers, renal cancer, bladder cancer, genitourinary cancer, esophageal carcinoma, large bowel cancer, metastatic cancers hematopoietic cancers, sarcomas, Ewing's sarcoma, synovial cancer, soft tissue cancers; and testicular cancer.

### F. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### Synergistic Up-Regulation of Plac8 by Oncogenic Mutations and the Cancer Phenotype

Malignant transformation arises from the sequential accumulation of multiple genetic mutations in normal cells that free the cell from the normal proliferation constraints, which results in uncontrolled, neoplastic growth. Analysis of gene expression changes as a result of malignant transformation in human tumor samples has been used to classify tumors, predict tumor behavior and determine whether primary tumors will metastasize. Genomic analysis can also identify gene expression changes associated with oncogene and tumor suppressor mutations in cancer. While the understanding of how the oncogenic activation of Ras and the loss of p53 tumor suppressor activity cooperate is incomplete, it is known that both activated Ras and p53 inactivation regulate the expression of many target genes, either directly or through signaling effectors. These studies have independently provided a great deal of information about activated Ras and p53 inactivation signaling respectively, however they have not addressed the potential cooperative regulation of gene expression by these oncogenes. Cooperating oncogenic mutations can affect multiple cancer cell traits, such as cell cycle progression and survival, which are mediated, at least in part, by gene expression changes. Matrix metalloprotease 9 (MMP9) was found to be cooperatively up-regulated by activated Ras and mp53. When MMP-9 expression was knocked-down in RasV12 and mp53 transformed cells via shRNAs, tumor formation in a xenograft model was significantly reduced and in vitro assays demonstrated an inhibition of invasiveness. Therefore, cooperatively regulated transcription targets exist in cells transformed by Ras activation and p53 inactivation and mediate specific biological properties required for the cancer phenotype. Identifying more of these cooperatively regulated genes presents an opportunity to discover new genes critical to the cancer phenotype.

To identify more cooperatively regulated genes on a genomic scale a micro-array analysis of YAMC, vector, mp53, RasV12 and mp53/Ras (transformed) cell polysomal RNA was conducted. This analysis revealed 538 differentially expressed genes (p<0.01; N-test, WestfallYoung adjusted) between mp53/Ras and YAMC control cells of which 95 annotated genes responded synergistically to mp53 and Ras. These 95 genes were termed “cooperation response genes”, in which 14 out of 24 of the genes significantly reduced tumor growth upon perturbation to YAMC levels, versus 1 out of 14 of non-synergistic differentially significantly reduced tumor growth, suggesting an enrichment of genes essential to the cancer phenotype in genes that are synergistically regulated by mp53 and Ras. Interestingly, 16% of the cooperation response genes are of unknown function, which presents an opportunity to identify novel genes involved in previously described properties of the cancer phenotype, or even novel biological properties specific to the cancer cell.

Cooperatively up-regulation of the gene Plac8, which is of unknown function, is essential to tumorigenicity of mp53/Ras cells and is the strongest inhibitor of tumor formation upon perturbation of the CRGs tested thus far. Also demonstrated herein is that Plac8 expression is essential to tumor formation of p53 inactivation and Ras pathway activation harboring human cancer cells lines HT-29, CAPAN-2, PANC-1 and Panc1 0.05, which also contain many other oncogenic mutations. These data demonstrate that Plac8 is an essential gene to the cancer phenotype regardless of oncogenic load or cell background and warrants further investigation into Plac8 function in cancer.

a) Plac8 is a Cooperatively Up-Regulated Gene at the Total and Polysomal RNA Level.

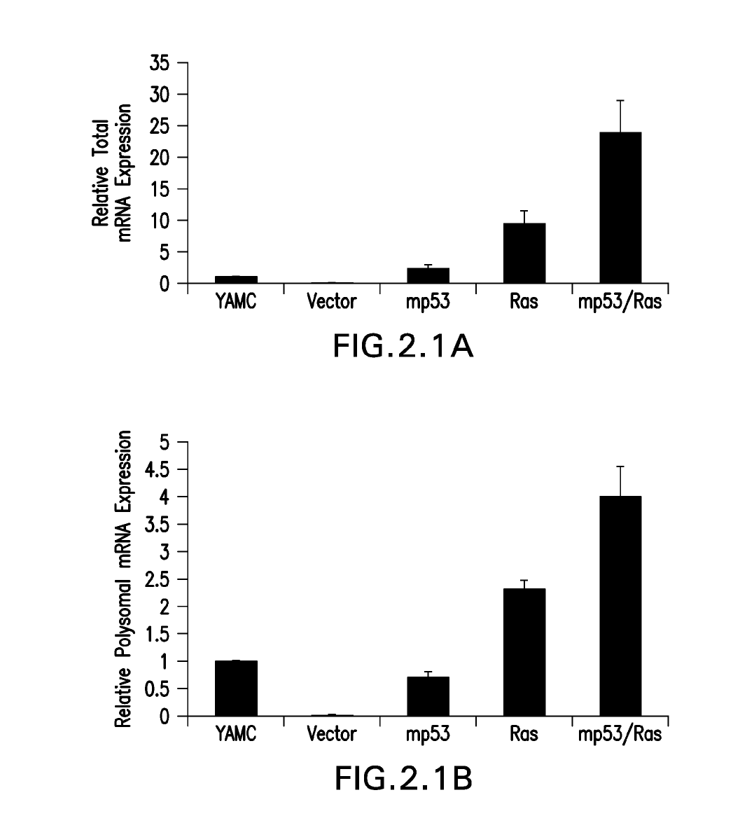

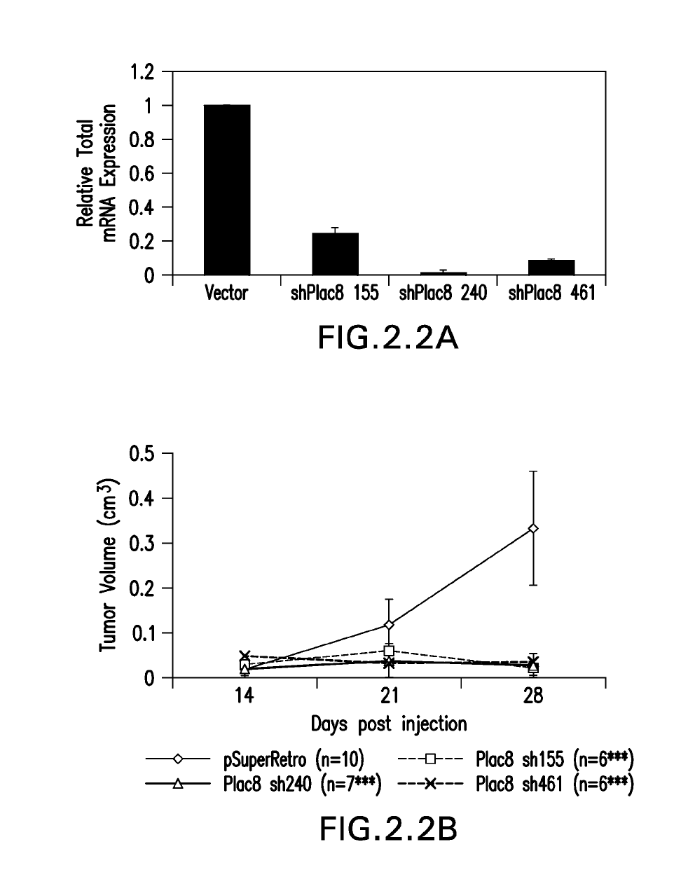

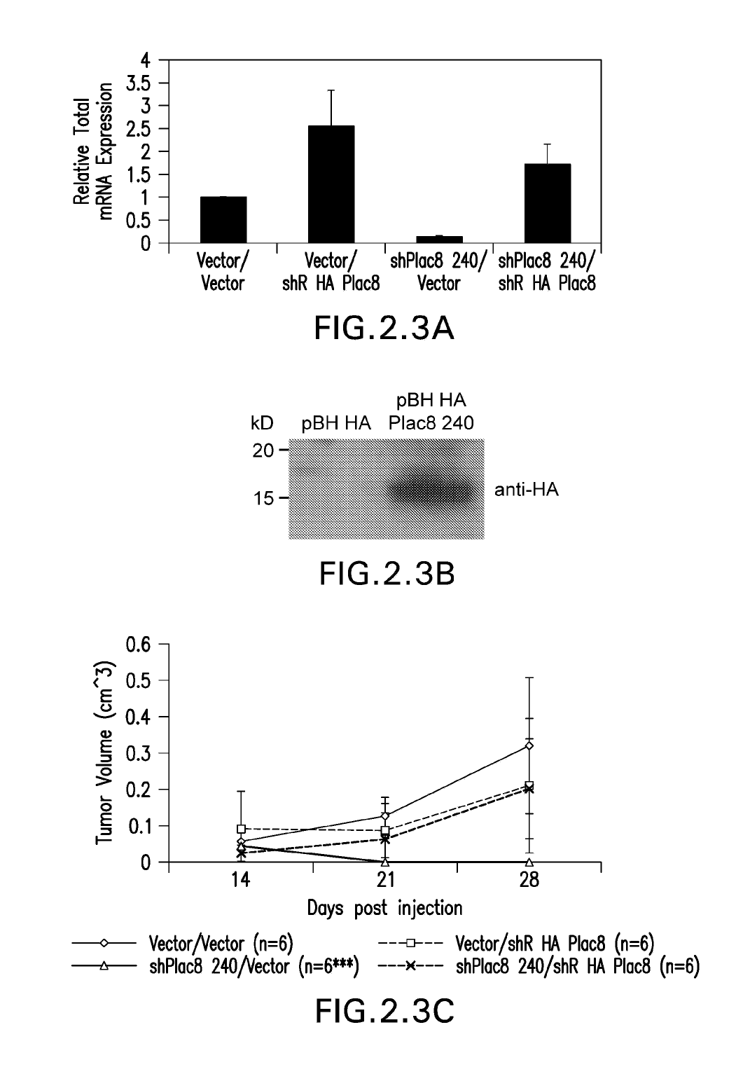

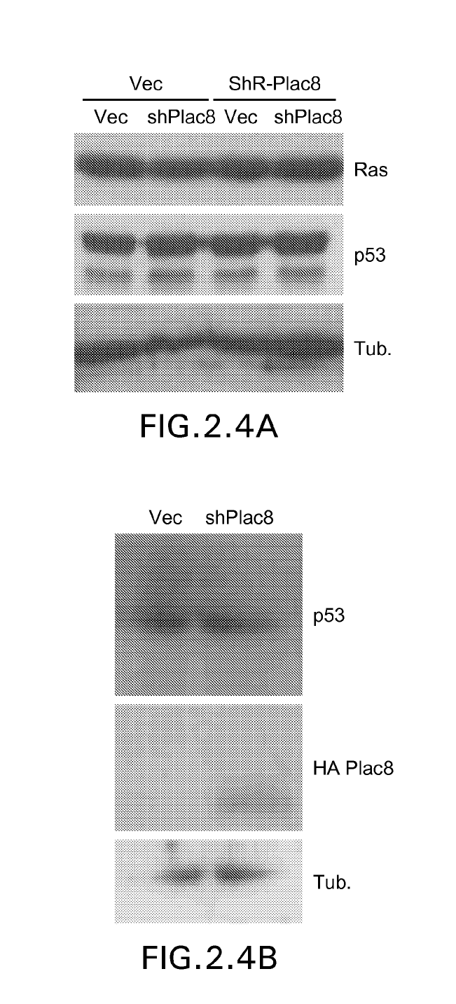

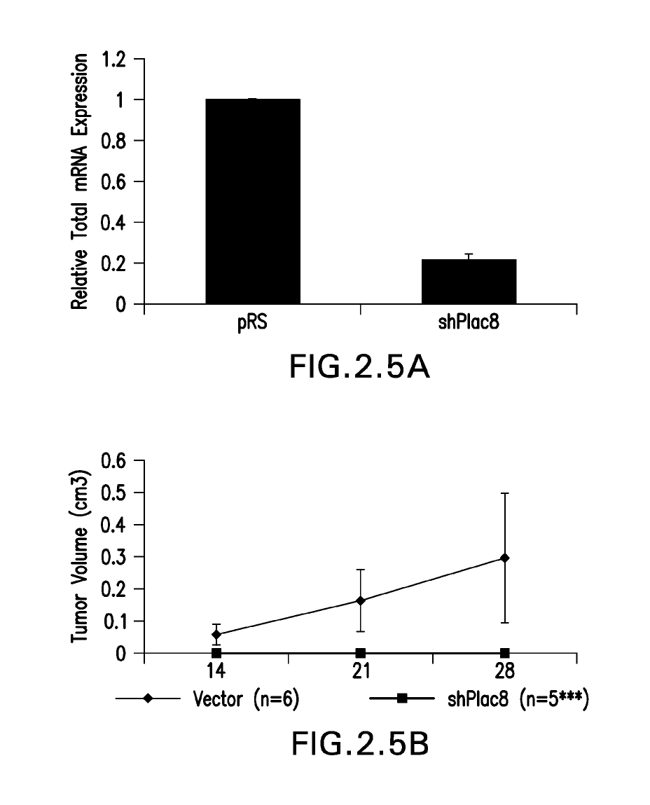

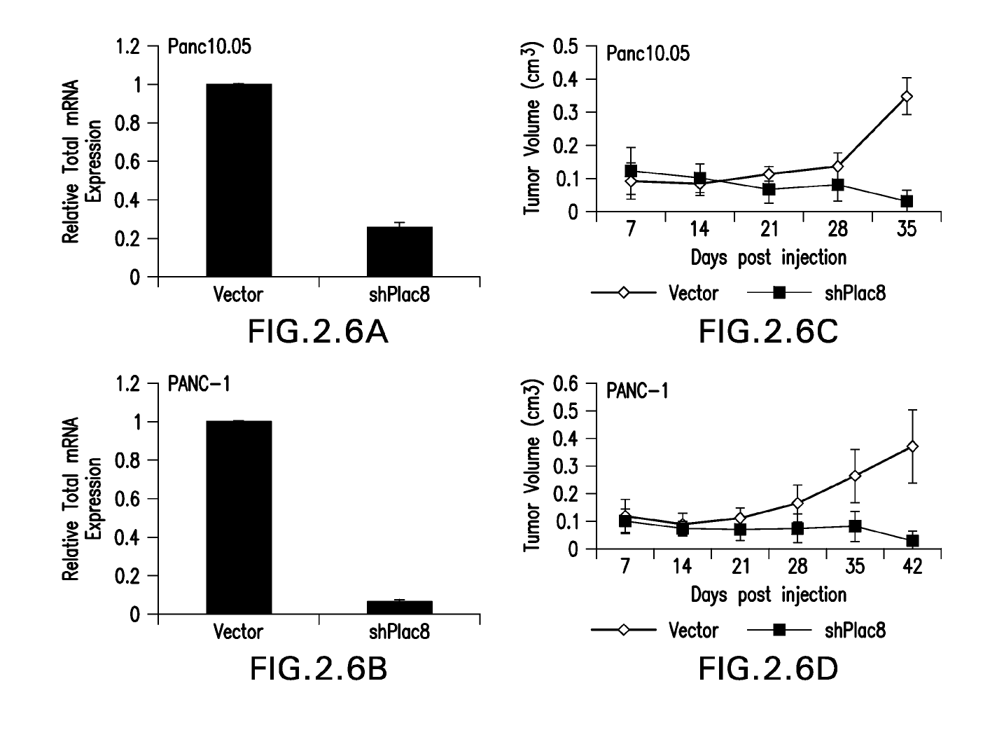

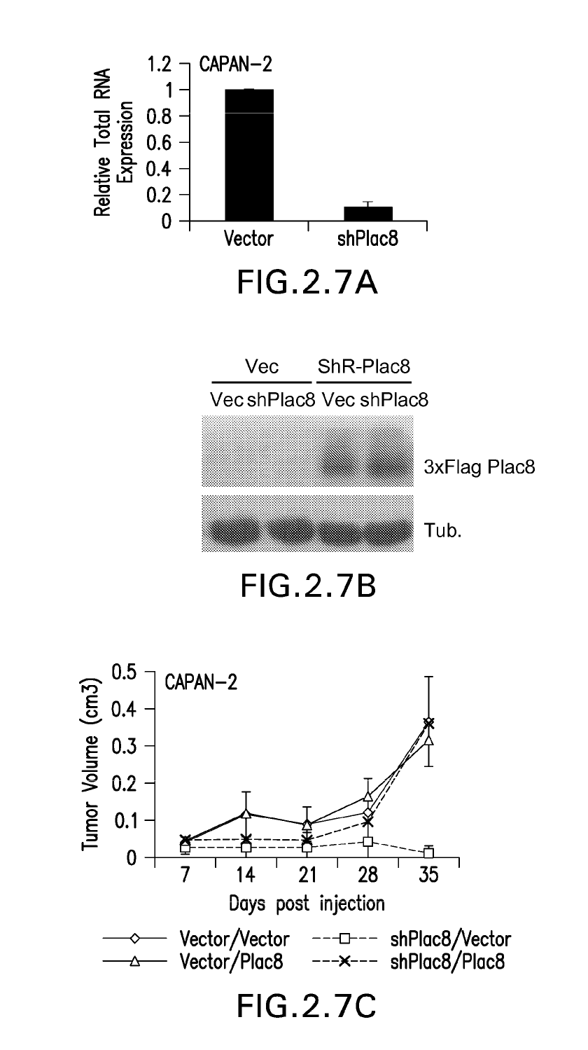

To independently confirm the synergistic up-regulation of Plac8 identified in the microarray analysis, reverse transcription and quantitative polymerase chain reactions (RT-qPCR) were conducted on total RNA samples from the parental, vector control, single oncogene and mp53/Ras (transformed) cells. The induction of Plac8 expression from YAMC to mp53, RasV12 and Transformed cells is 2.3, 9.4, and 23.8 fold respectively in total RNA samples (FIG. 2.1a). Plac8 responds to both oncogenic mutations alone, most notably Ras, but is synergistically up-regulated by oncogene cooperation as observed in the micro-array analysis (FIG. 2.1b).

b) Cooperative Up-Regulation of Plac8 is Required for Tumor Formation

To characterize the contribution of the cooperatively up-regulated genes to the transformed state their expression was perturbed by siRNA-mediated knock-down of gene expression. Gene expression perturbation is accomplished with the pSuperRetro system, which encodes for the production of short hairpin RNAs (shRNA) via the H1 Pol-III promoter. To identify if the plac8 gene is important for the transformed state the expression Plac8 was perturbed with three independent knock-down constructs targeting distinct nucleotide sequences to control for off-target effects (FIG. 2.2a). The shPlac8 155, 240 and 461 siRNA constructs can knock-down Plac8 total RNA expression levels to 76%, 99% and 92% of vector control levels quantitated by real-time qPCR, respectively.

To explore how a gene perturbation affects the cancer phenotype, mp53/Ras transformed cells harboring plac8 gene perturbations or respective vector controls were injected into nude mice to determine tumor formation capacity. In vivo tumor formation data for Plac8 knock-down demonstrates that upon injection into nude mice, tumor formation was significantly inhibited compared to vector control (FIG. 2.2b). These data indicate that Plac8 loss-of-function results in loss of tumor formation.

shRNA mediated knock-down can also perturb mRNA transcript expression of genes with similar sequences to the target gene, which can result in non-specific, off-target effects. Specificity of the Plac8 shRNA mediated loss of tumor formation was confirmed via Plac8 genetic rescue. This experiment was conducted by introducing silent mutations in the Plac8 gene via site-directed mutagenesis for resistance to the shRNA. To identify if a protein is produced a HA-epitope was also added to the N-terminus of the protein. Plac8 RNA expression was confirmed by RT-qPCR analysis conducted on total RNA from Plac8 knock-down, Plac8 rescue and vector control cells (FIG. 2.3a). Moreover, production of the HA tagged protein was demonstrated by immunoblotting of cell extracts for the HA epitope, revealing a protein of the expected molecular weight at approximately 14 kD (FIG. 2.3b). Upon injection into nude mice the vector control, Plac8 over-expressing, and Plac8 rescue cells all form tumors, where as the Plac8 knock-down cells do not after 4 weeks (FIG. 2.3c).

Plac8 up-regulation is a down-stream event of p53 loss-of-function and Ras activation. Nevertheless, it is possible that Plac8 affects mp53 or Ras protein levels. It has been previously demonstrated that Plac8 over-expression in Rat1a cells can induce p53 degradation. To test this possibility Plac8 knock-down, Plac8 over-expression, Plac8 rescue and vector control cells for p53 and Ras were immunoblotted, which showed that mp53 and Ras protein levels were unchanged between these cell lines (FIG. 2.4a), indicating that Plac8 functions downstream of mp53 and Ras. To test if endogenous p53 is degraded by Plac8 over-expression, Plac8 was over-expressed in Ras cells and immunoblotted against p53 (FIG. 2.4b). p53 protein levels are not changed by Plac8 over-expression, indicating that that Plac8 does not increase p53 degradation in the cells. These data thus indicate that tumor formation requires Plac8 downstream of the oncogenic mutations in mp53/Ras transformed cells and Plac8 does not contribute to p53 loss-of-function dependant transformation.

c) Plac8 is Required for Tumor Formation Regardless of Oncogenic Load or Cell Background.

Nearly all human colorectal adenocarcinoma cell lines contain oncogenes c-myc, H-ras, K-ras, N-ras, myb, fos and p53 oncogenic mutations, while pancreatic adenocarcinoma cell lines typically contain K-ras, p53, p161NK4, DPC4 and FHIT mutations. These extra oncogenic mutations render tumor formation independent of Plac8 expression in human cancer. Two of the three independent human Plac8 siRNA targets were verified to knock-down Plac8 total RNA expression in HT-29 cells to 89% compared to vector control by real-time qPCR (FIG. 2.5a). Moreover, in contrast to vector controls, these Plac8 knockdown cell lines do not form tumors (FIG. 2.5b). To test the effects of Plac8 perturbation in other types of human cancer cells Plac8 was knocked-down in the pancreatic adenocarcinoma cell lines CAPAN-2, PANC-1, and Panc1 0.05 to 90%, 94%, and 74% as compared to vector control, respectively (FIG. 2.6a,b; FIG. 2.7a) and evaluated tumor formation capacity upon transplanting the cells into immunocompromised nude mice (FIG. 2.6c,d; FIG. 2.7c). Plac8 knock down inhibited tumor formation in all of these cell lines. Moreover, inhibition of tumor formation by Plac8 knock down was rescued in CAPAN-2 cells by ectopic expression of murine 3× Flag-tagged Plac8, which is resistant to the human Plac8 targeting shRNA (FIG. 2.7b,c). These data indicate that Plac8 is required for tumor formation in pancreatic and colorectal adenocarcinoma cell backgrounds and is required for the cancer phenotype regardless of the presence of oncogenic mutations in addition to mutant Ras and mutant p53.

d) Summary

Malignant transformation by mp53 and Ras induces synergistic changes in gene expression that are enriched in genes essential for the cancer phenotype. The expression of one of these genes, Plac8, is synergistically up-regulated by mp53 and Ras, and has the largest inhibitory effect on tumor formation when its expression is readjusted to YAMC cell levels via shRNA mediated knock-down in mp53/Ras cells, and can be rescued by expression of an shRNA-resistant Plac8. This is downstream of the oncogenic mutations, because Plac8 shRNA-mediated knock-down has no effect on Ras or mp53 protein in transformed cells. Thus, synergistic up-regulation of Plac8 is essential to the cancer phenotype downstream of the initiating oncogenic mutations. Plac8 over-expression was previously described to transform Rat1a cells by inducing p53 degradation through Akt and Mdm2 activation, however, a change in p53 protein levels in Plac8 KD or over-expression transformed cells and in Plac8 over-expressing Ras cells was not observed herein. Moreover, in mp53/Ras transformed cells p53 function is deactivated indicating that Plac8 must have an essential function independent of p53 in malignant cells.

Plac8 was also required in both human colorectal and pancreatic adenocarcinoma cell lines, indicating that the malignant state is dependent on Plac8 in multiple cell backgrounds. Moreover, HT-29, PANC-1, CAPAN-2, and Panc10.05 human cancer cell lines carry multiple oncogenic mutations in addition to p53 and Ras or Raf, indicating additional oncogenic mutations cannot compensate for Plac8 loss of function in malignant cells. These data indicate that Plac8 has a novel function that is essential to the cancer phenotype in a variety of contexts, and warrants further investigation into Plac8 function in cancer.

### 2. Example 2

### Plac8 is an Internal Lysosomal Protein Required for Autophagosomal/Lysosomal Fusion

Genes cooperatively regulated by mp53 and Ras are enriched for genes essential to the cancer phenotype. Out of the cooperation response genes currently known to contribute to the cancer phenotype Plac8 perturbation has the largest inhibitory effect on tumor formation, and is required for human colorectal and pancreatic adenocarcinoma cell line tumorigenicity, but is of unknown function in cancer. These data detailed herein demonstrated that Plac8 is an essential gene to the cancer phenotype in the presence of Ras, p53 and other oncogenic mutations in various cell backgrounds. This strict requirement for plac8 expression for the cancer phenotype prompted in depth investigation into Plac8 function in cancer. Herein is shown that Plac8 is an internal lysosomal protein that is required for autophagosomal/lysosomal fusion and ultimately completion of the autophagy process.

Autophagy or “self-eating” is a strategy for cells to survive under metabolic stress by degrading damaged macromolecules and organelles to recycle metabolites for energy and anabolism. Autophagy was initially thought to suppress tumor formation due to an increase in tumorigenesis in transgenic mice with heterozygous knock-out of Beclin1, a protein that is involved in autophagosome formation, as well as monoallelic inactivating mutations in the Beclin1 activated complex protein UVRAG in a variety of human cancers, suggesting that inhibition of autophagy promoted malignant transformation. It was also demonstrated that KO of ATG5, another gene involved in autophagosome formation, with over-expression of the anti-apoptotic gene Bcl-2 in iBMK cells enhanced tumorigenicity, again indicating that elimination of autophagy genes promoted tumor formation, suggesting that the autophagy process was tumor inhibitory.

Recent data suggested that autophagy has a role in promoting cancer. Loss or mutation of p53, which occurs in almost 50% of all cancers, by shRNAmediated p53 KD, p53 KO or introduction of p53 R1 75H induces autophagy and re-introduction of WT p53 into p53 KO HCT116 colorectal adenocarinoma cells suppressed autophagy. An increase in autophagy has also been shown to protect cancer cells from metabolic stress induced by chemo- and radiotherapy, as well as metabolic stress from poor vasculature, which typically results in necrosis. To reconcile the seemingly conflicting data it has also been suggested that both too much or too little autophagy can be detrimental to the cancer cell and there is an optimal rate at which autophagy is required for the cancer phenotype, however no data has been provided thus far to validate or negate this hypothesis.

a) Plac8 is a Cooperatively Up-Regulated, Internal Lysosomal Protein that is Induced by Hypoxia and Nutrient Starvation Indicating a Role in Autophagy.

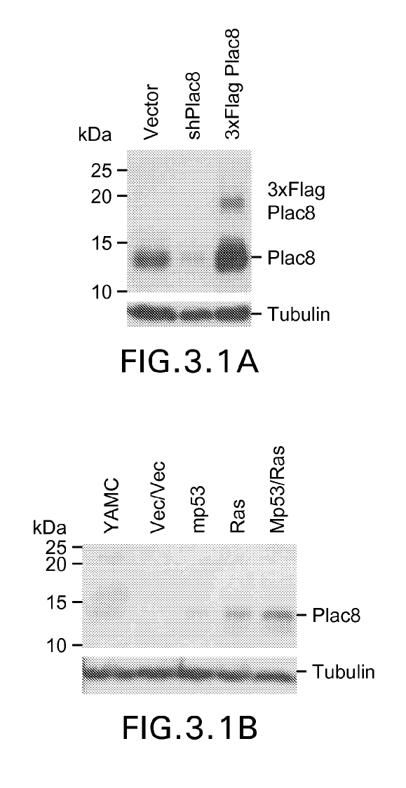

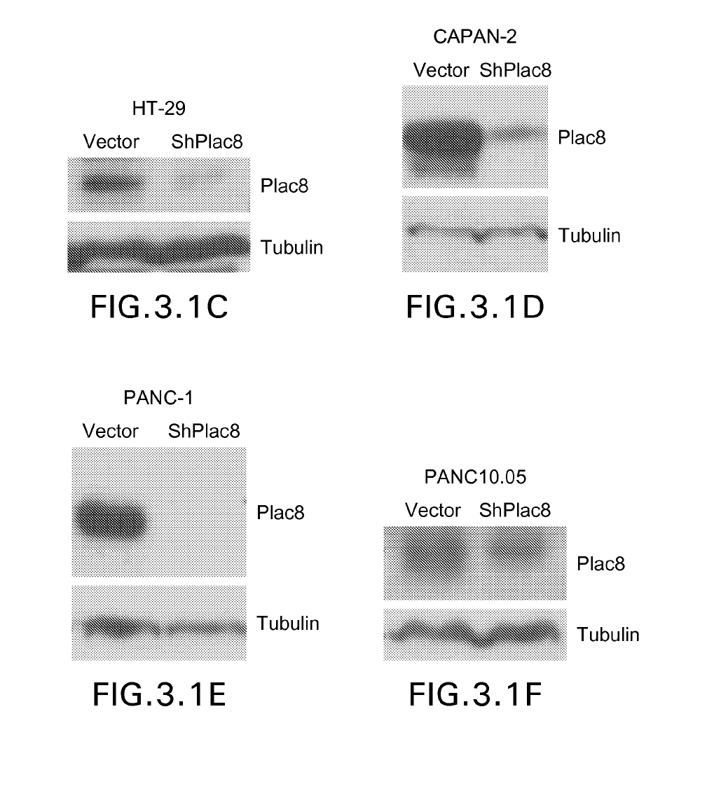

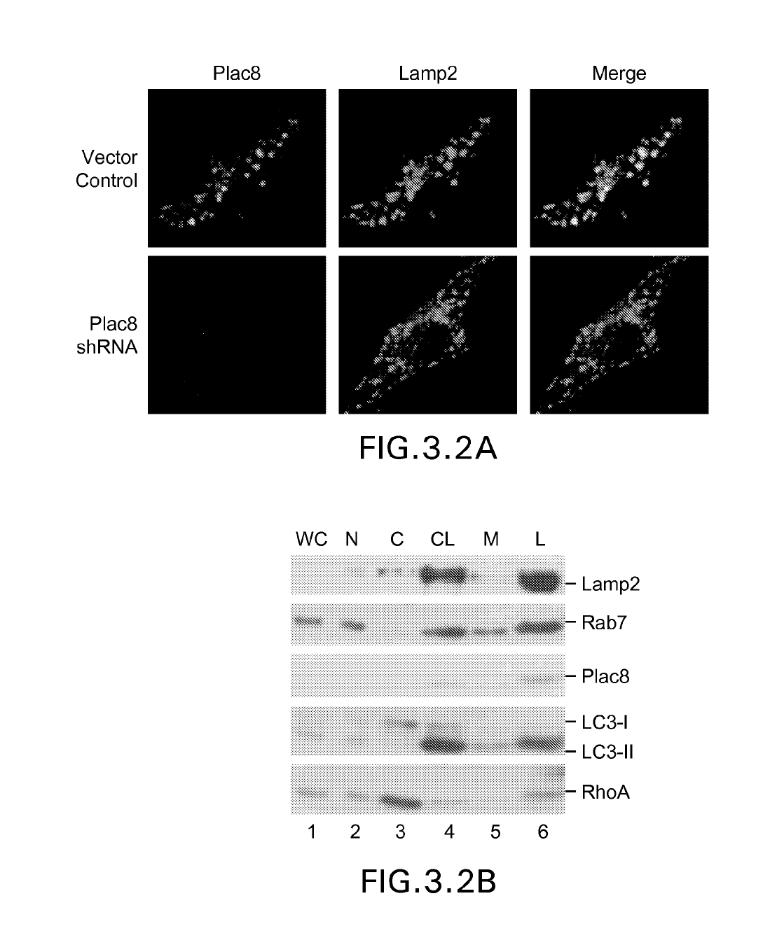

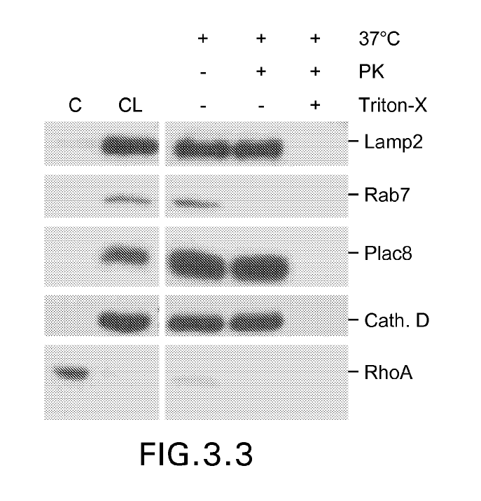

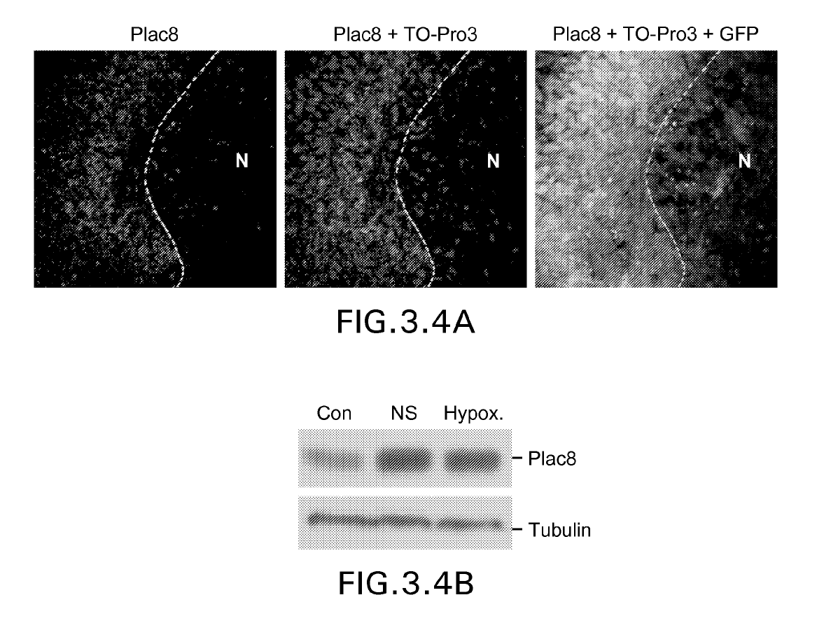

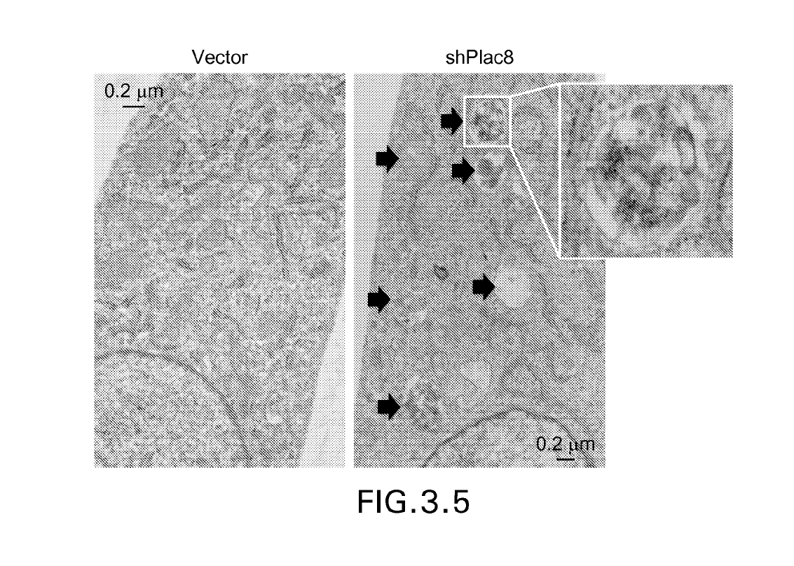

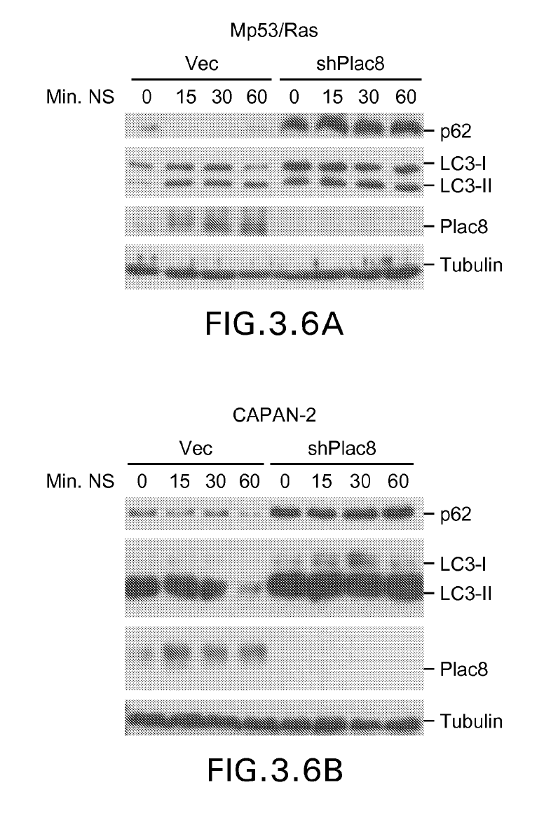

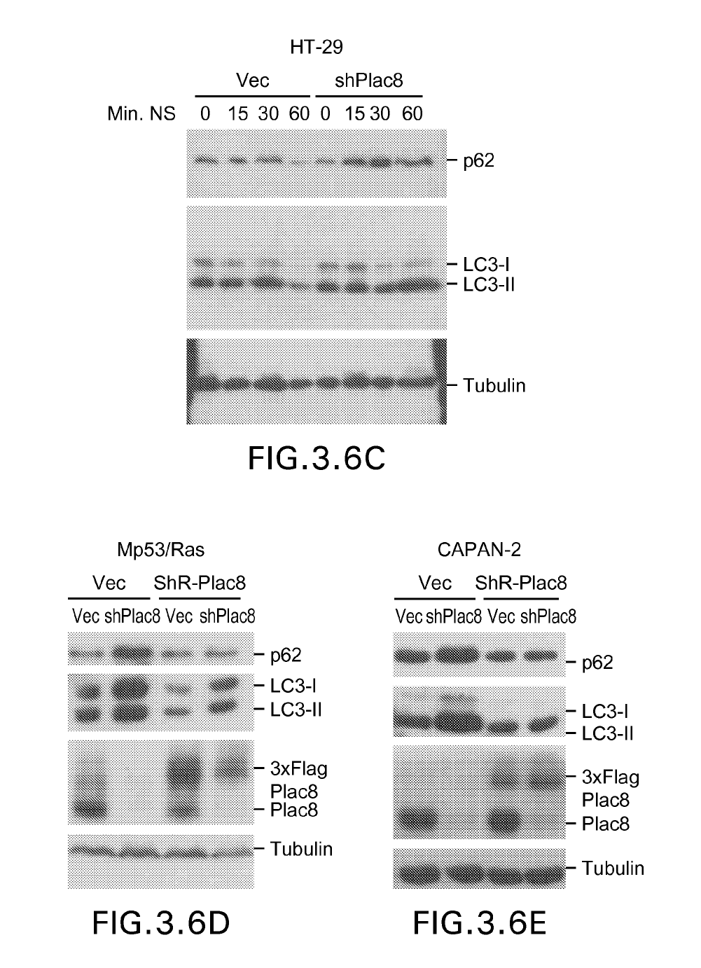

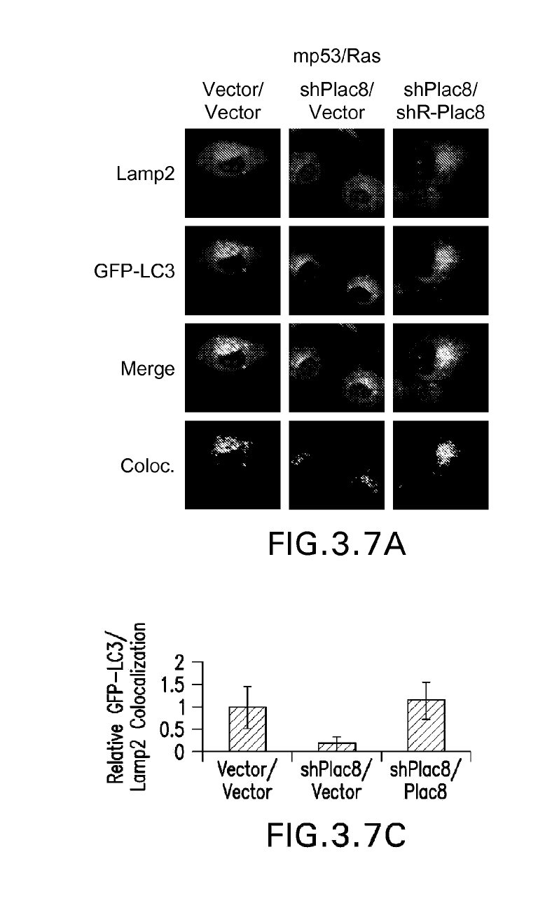

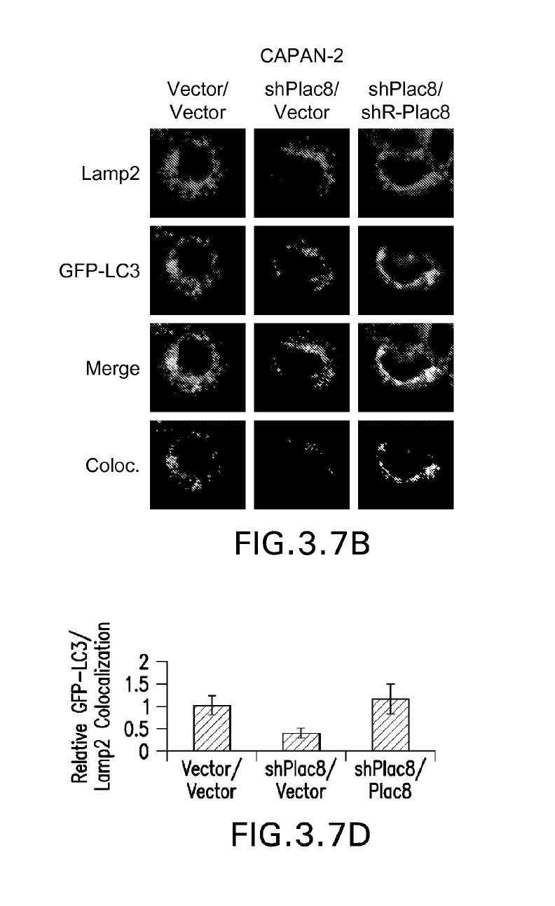

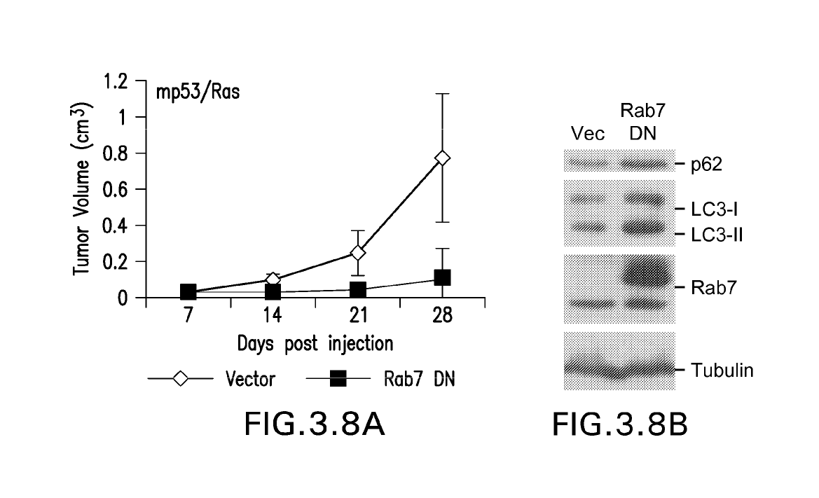

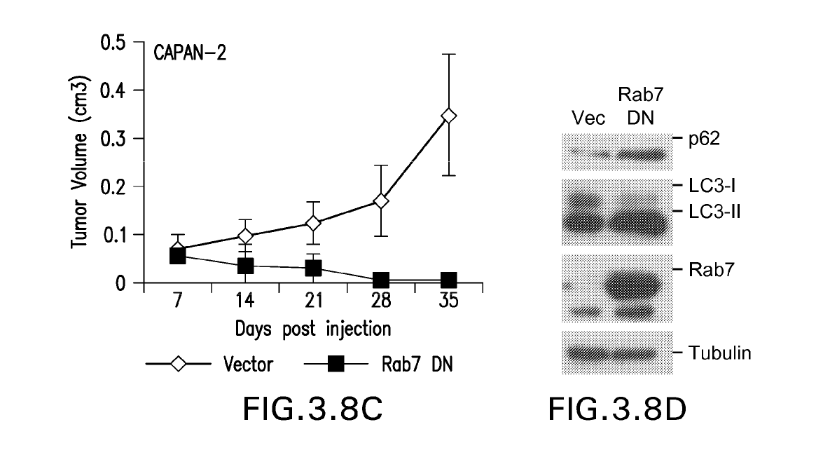

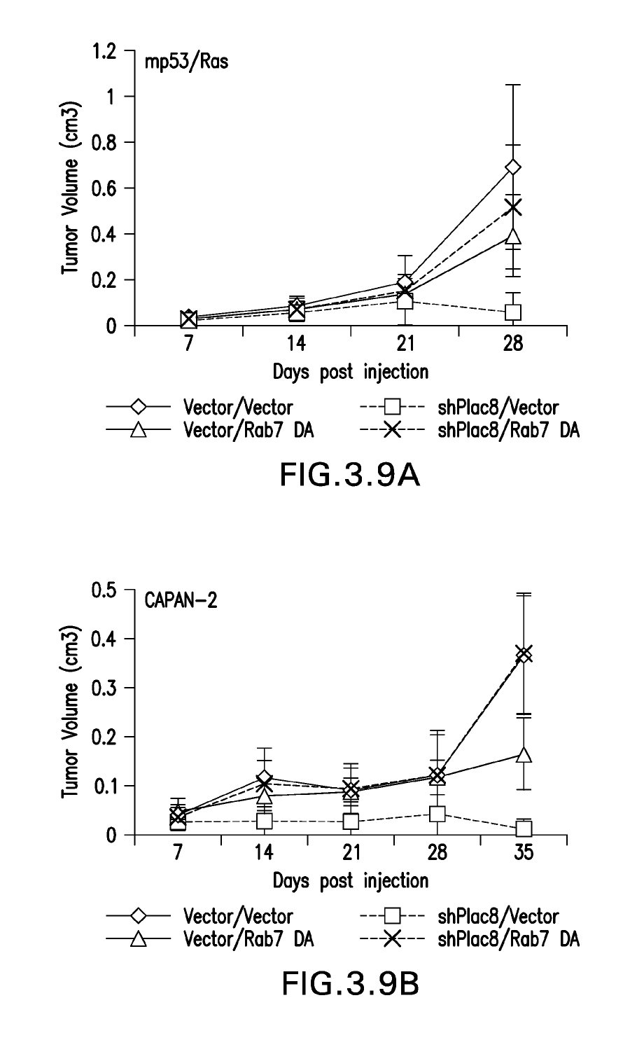

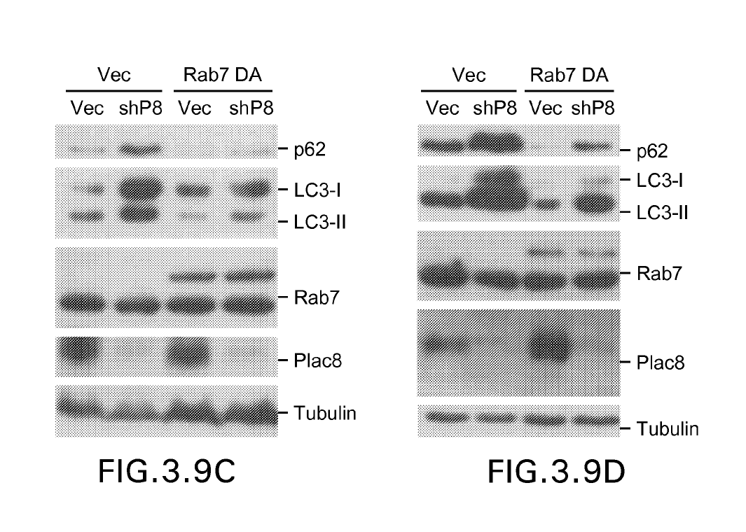

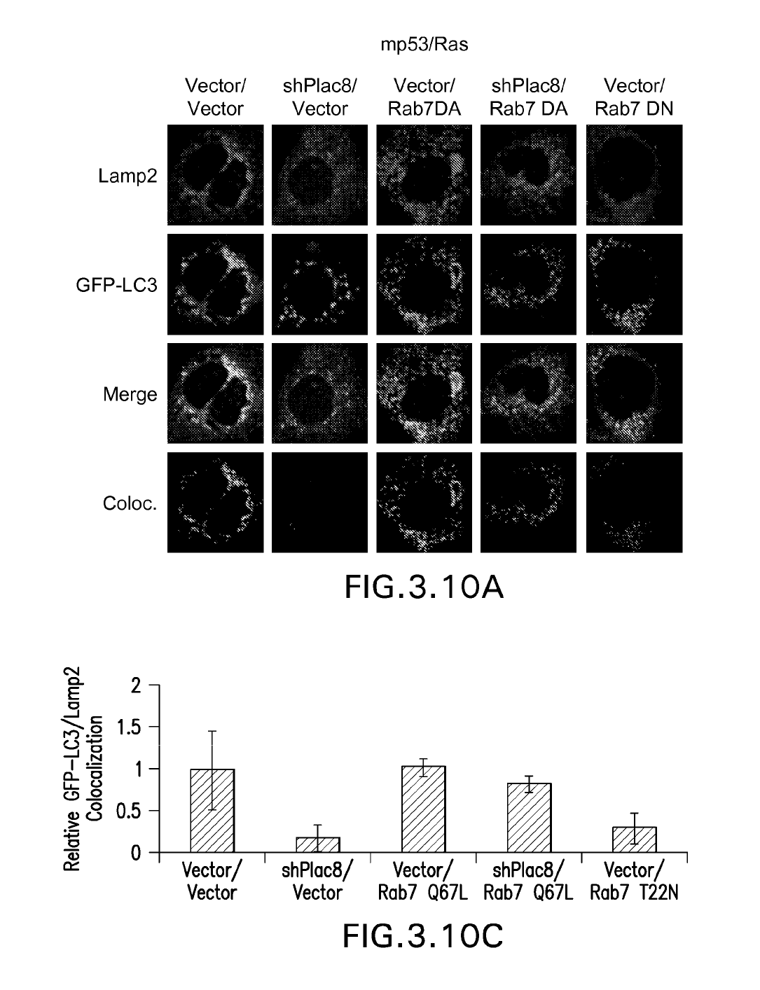

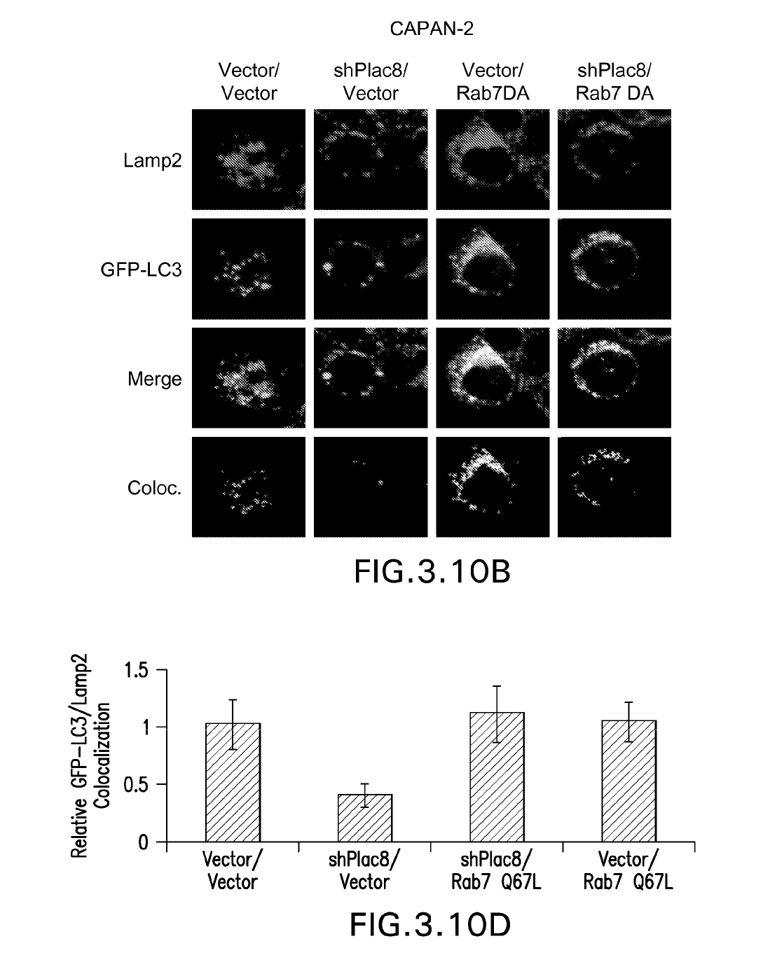

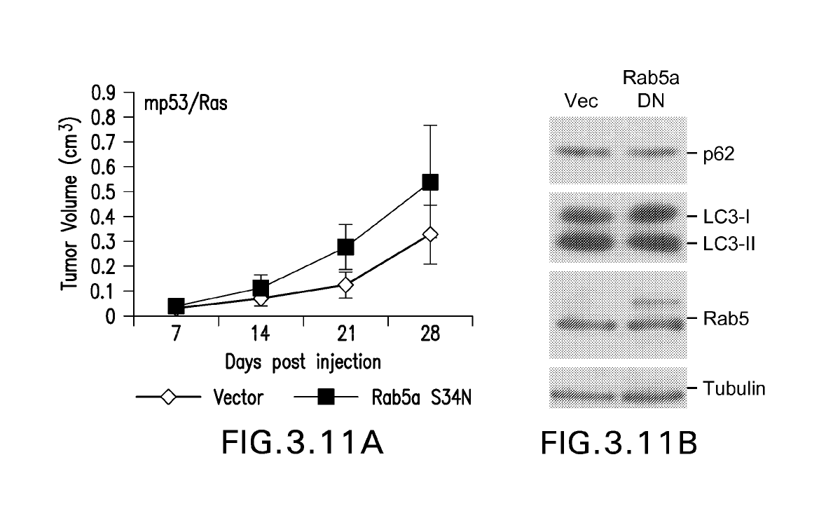

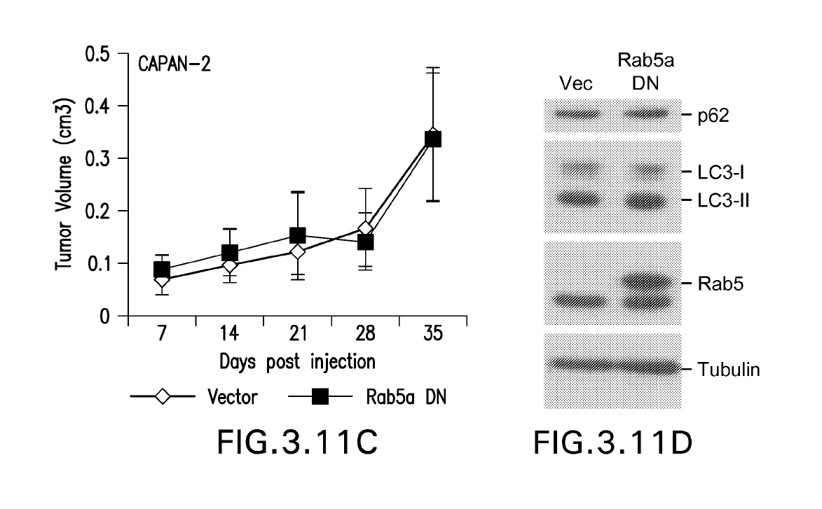

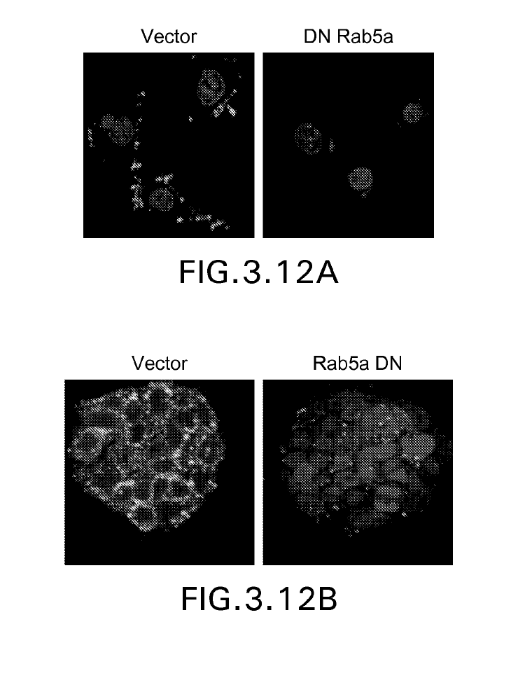

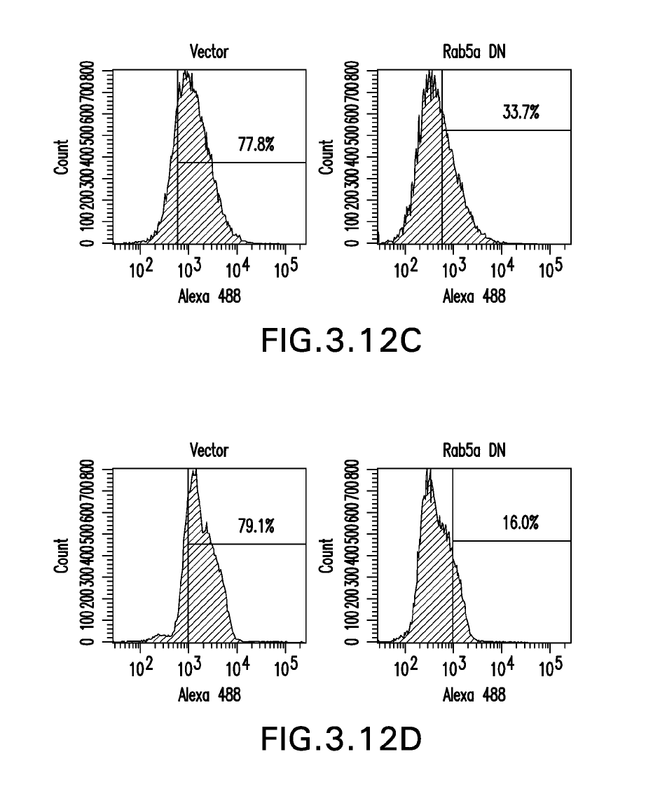

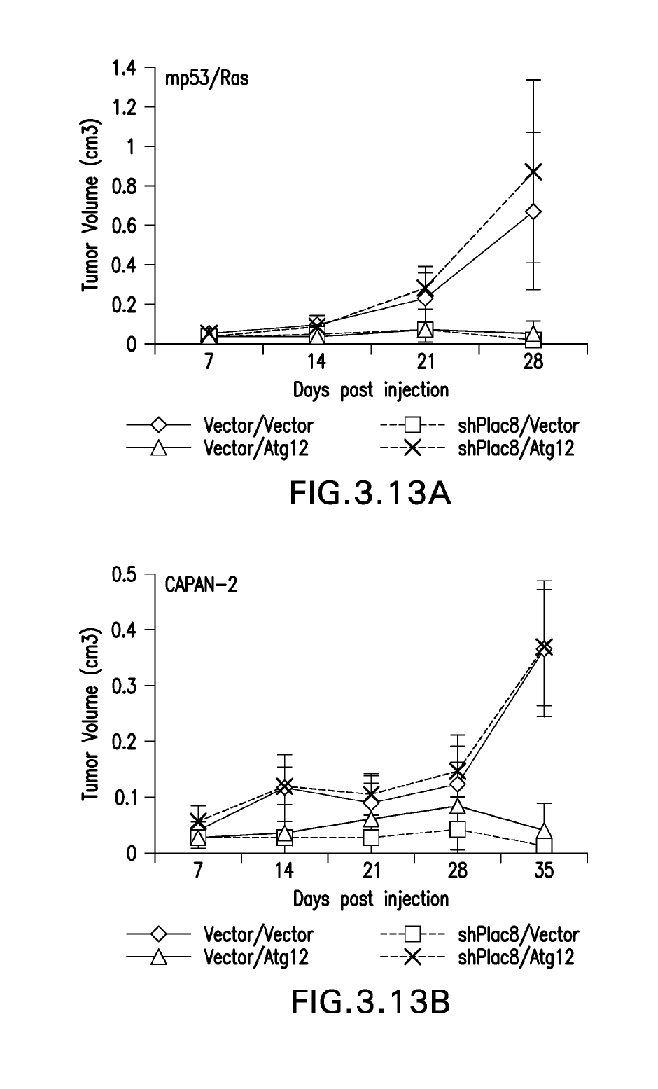

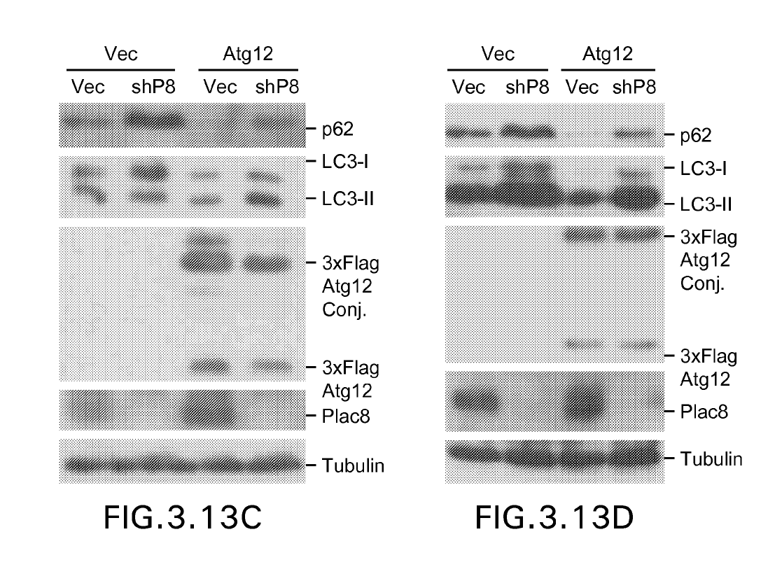

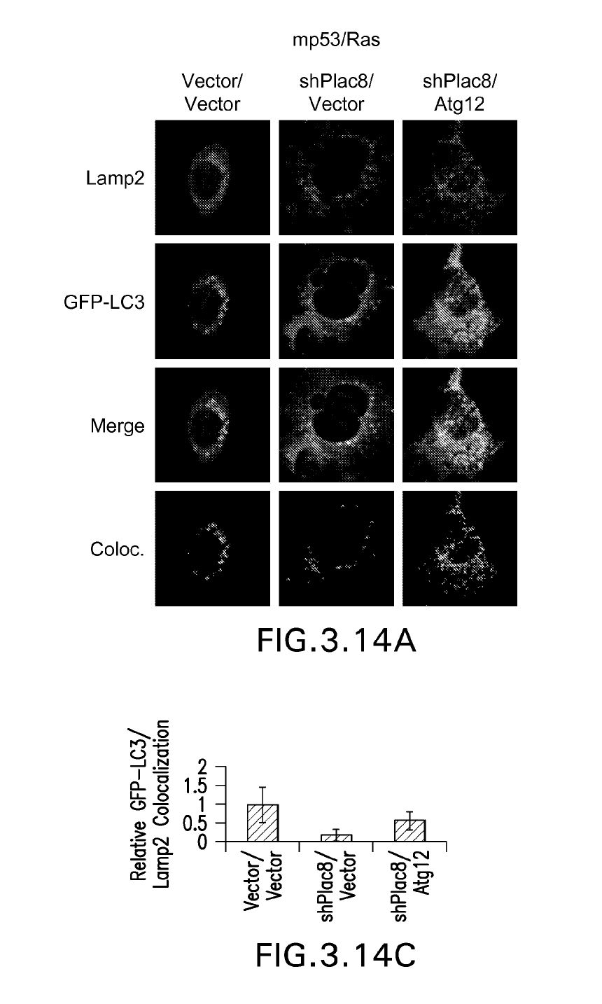

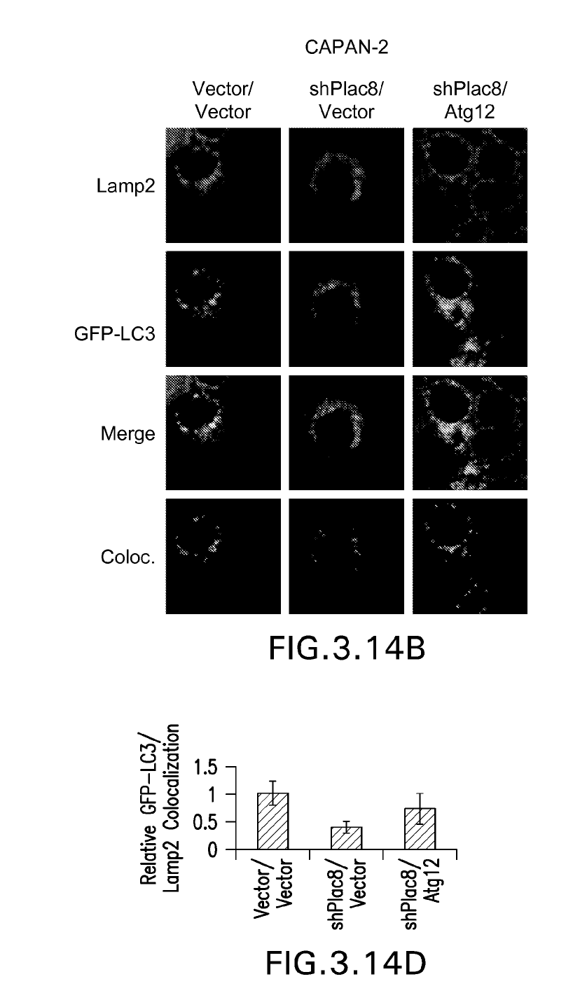

To explore in what process Plac8 is involved, a Plac8 antibody was generated by immunizing rabbits with the C-terminal 16 amino acids of the murine Plac8 protein conjugated to KLH. The antibody recognized a 14 kDa protein knocked-down by Plac8 shRNA, as well as, an exogenously expressed Plac8 protein (FIG. 3.1a) in a manner consistent with similar analyses reported by Ledford et al. The antibody also recognized the human form of Plac8, which is knocked-down by Plac8 shRNA (FIG. 3.1b-d). As observed for plac8 polysomal RNA, the Plac8 protein also shows a cooperative up-regulation by Mp53 and RasV12 (FIG. 3.1b). To determine the sub-cellular localization of the endogenous Plac8 protein, Mp53/RasV12 cells were immunostained with the Plac8 antibody, which showed a punctate staining that partially co-localized with the lysosomal protein Lamp2, indicating lysosomal compartmentalization (FIG. 3.2a).

To confirm lysosomal localization of Plac8 lysosomes were isolated by sub-cellular fractionation and density centrifugation, which showed an enrichment of Plac8 protein together with the known lysosomal proteins Lamp2 and Rab7 in the lysosomal fractions (FIG. 3.2b). The Plac8 protein has also been previously described to be enriched in the granular fraction of neutrophils, which is a modified form of lysosome. To determine whether Plac8 is an internal or external lysosomal protein lysosomes were isolated and exposed them to Proteinase K or Proteinase K plus Triton-X (FIG. 3.3). The Plac8 protein is protected from degradation by Proteinase K similar to the internal lysosomal proteins Lamp2 and CathepsinD, whereas the external lysosomal protein Rab7 is degraded, indicating that the Plac8 protein is an internal lysosomal protein.

To further identify a role for Plac8 in the cancer phenotype the expression of Plac8 was explored in mp53/RasV12 tumors that were labeled with GFP to unequivocally identify injected tumor cells. Plac8 expression was visualized via immunofluorescence by staining tumors with the Plac8 antibody and was found induced around areas of tissue showing nuclear deficiency indicative of necrosis (FIG. 3.4a). Such areas have previously been shown to up-regulate autophagy.

Autophagy is a cellular defense mechanism for metabolic stresses such as nutrient starvation and hypoxia. Proteins involved in the autophagy process are commonly up-regulated under these conditions. For example, the internal lysosomal protein Lamp2 has been previously shown to be induced by hypoxic and nutrient starvation stress, due to a role in autophagy. Therefore Mp53/RasV12 cells were exposed to hypoxic and nutrient starvation stress, which resulted in the accumulation of Plac8 protein indicating a possible role in autophagy (FIG. 3.4b). Ledford et al. also demonstrated that KO of Plac8 in neutrophils inhibited intracellular bacterial killing, indicating a possible defect in phagosome maturation, which employs the same components as autophagosome maturation. These data taken together indicated a possible role for Plac8 in the process of autophagy.

b) Plac8 KD Results in an Accumulation of Autophagosomes and Autophagosomal Markers from an Inhibition of Autophagosomal/Lysosomal Fusion.

To explore if Plac8 knock-down (KD) affects autophagy Mp53/RasV12 vector control and Plac8 KD cells were compared by transmission electron microscopy to determine changes in autophagy by ultra-structural identification of autophagosomal structures. This revealed an accumulation of autophagosomes in Plac8 shRNA-mediated KD cells over vector control (FIG. 3.5). Plac8 KD also resulted in the accumulation of the biochemical autophagic markers p62 and LC3 in murine mp53/RasV12 (FIG. 3.6a), as well as human Capan-2 (FIG. 3.6b) and HT-29 (FIG. 3.6c) cancer cell lines after 60 minutes of nutrient starvation. Notably, p63 and LC3 were restored to similar protein levels found in vector control cells upon expression of an shRNA-resistant Plac8 protein in both mp53/Ras and Capan-2 cells lines (FIG. 3.6d, 3.6e). The accumulation of autophagosomes and autophagosomal markers indicates that Plac8 KD results in a change in the rate of the autophagy process, either a stimulation or inhibition.

The Plac8 KD dependent accumulation of autophagosomes and autophagosomal biochemical markers can be due to an induction of autophagosome formation (on-rate) or inhibition in autophagosome clearance via autophagosomal/lysosomal fusion (off-rate). The specific accumulation of p62 and LC3-II, which is specifically degraded upon lysosomal fusion, indicates a block in autophagosomal/lysosomal fusion. To explore if Plac8 is required for autophagosomal/lysosmal fusion a GFP-LC3 fusion protein was expressed to label autophagosomes and immunostained for Lamp2, a lysosomal marker. Co-localization of GFP-LC3 and Lamp2 indicates the formation of an autolysosome, or the completion of autophagosomal/lysosomal fusion. Plac8 KD results in a 70-80% reduction in GFP-LC3/Lamp2 co-localization compared to vector control, which was rescued by the expression of an shRNA-resistant Plac8, thus indicating that Plac8 promotes autophagosomal/lysosomal fusion (FIG. 3.7a-d).

c) Rab7 Activity is Required for Tumor Formation and Constitutive Activation of Rab7 Rescues Plac8 KD Tumor Formation Inhibition.

To determine if the autophagosomal/lysosomal fusion is the tumor essential process for which Plac8 is required, a determination was made as to whether the autophagosomal/lysosomal fusion process was tumor inhibitory. Autophagosomal/lysomal fusion is also controlled, at least in part, by the Ras-like GTPase Rab7 (Gutierrez, et al., 2004; Jager, et al., 2004), and expression of the Rab7 T22N dominant negative mutant (Rab7 DN) has been shown to inhibit autophagosomal/lysosomal fusion (Gutierrez, et al., 2004; Jager, et al., 2004). Expression of Rab7 DN resulted in an inhibition of tumor formation and accumulation of p62 and LC3, phenocopying Plac8 KD (FIG. 3.8a-3.8d). Moreover, the tumor-inhibitory effect of Plac8 knock down and the accumulation of p62 and LC3 following Plac8 KD was reversed by the over-expression of the Rab7 Q67L dominant active mutant (Rab7 DA) (FIG. 3.9a-d), that activates autophagosomal/lysosomal fusion. Thus, the effects of Plac8 KD on both tumor formation and autophagy can be suppressed by constitutive activation of Rab7. GFP-LC3/Lamp2 colocalization was also inhibited by the expression of Rab7 DN (FIG. 3.10a-d) and loss of GFP-LC3/Lamp2 colocalization mediated by Plac8 KD was rescued by expression of Rab7 DA indicating the inhibition of autophagosomal/lysosomal fusion by Plac8 KD can be rescued by activated Rab7. Furthermore these data indicate that autophagosomal/lysosmal fusion is required for the cancer phenotype.

Rab7 in-activation inhibits tumor formation, however, expression of a DN Rab protein non-specifically disrupts endosomal trafficking, which requires multiple different Rab proteins, and thereby result in tumor formation inhibition independent of the autophagy process. Moreover, the molecular machinery employed in autophagosomal/lysosomal fusion, such as Rab7, is required for endosomal/lysosomal fusion, which is also required for tumor formation in some cancers. To determine if the inhibition of tumor formation and autophagic marker accumulation is specific to Rab7 in-activation and autophagosomal/lysosomal fusion a Rab5a dominant negative mutant was expressed, which has been shown to inhibit early endocytosis/phagocytosis, thereby dissecting the contribution of the endocytic process from the autophagic process. The expression of Rab5aDN increased tumor size over vector control (FIG. 3.11a, 3.11c) and had no effect on p62 or LC3 levels (FIG. 3.11b, 3.11d), indicating that Rab5a inactivation does not have effect autophagy or tumor formation capacity of malignant cells. Rab5a inactivation did inhibit endocytosis/phagocytosis, as indicated be a decrease in uptake of fluorescently labeled dextran in mp53/Ras transformed and CAPAN-2 cells (FIG. 3.12a-d), indicating that in these cell lines the inhibition of endocytosis does not inhibit the cancer phenotype.

d) Over-Activation of Autophagy by Atg12 Overexpression Rescues Plac8 KD

That both Plac8 function and Rab7 activity are required for autophagosomal/lysosomal fusion and tumor formation has been demonstrated herein, however, Plac8 KD-mediated inhibition of tumor formation can be due to another lysosomal process essential to the cancer phenotype. To determine if the Plac8 KD-induced loss of tumor formation is specifically due to a loss of autophagy completion, Atg12, a gene required for autophagosomal formation, was overexpressed in Plac8 KD cells. Atg12 overexpression restored tumor formation capacity to Plac8 KD cells, but surprisingly Atg12 overexpression was tumor inhibitory without Plac8 KD, indicating an epistatic interaction between Atg12 and Plac8 (FIG. 3.13a, 3.13b). Western blot of the autophagic markers p62 and LC3 revealed that p62 and LC3 protein levels were suppressed by overexpression of ATG12, but p62 and LC3-I are restored to vector levels by KD of Plac8 (FIG. 3.13c, 3.13d). LC3-II levels are higher in Atg12 over-expression/Plac8 KD cells then vector control indicating that even though autophagy is induced the process is restricted at the point of degradation. Analysis of the autophagosomal/lysosomal fusion demonstrated that Atg12 rescue of Plac8 KD resulted in more colocalization of GFP-LC3 and LAMP2 (FIG. 3.14a-d), indicating that an increase in autophagosome formation can compensate for an inhibition in autophagosomal/lysosomal fusion. These data indicate that Plac8 is specifically required for autophagosome maturation, and Plac8 KD inhibition of tumor formation results from the inhibition of the autophagy process.

e) Summary

Plac8 is a cooperatively up-regulated gene by mp53 and Ras and is required for tumor formation in multiple cancer cell lines. However, Plac8 function in cancer is unknown. Herein is disclosed that Plac8 is an internal lysosomal protein through immunofluorescent partial co-staining of Plac8 and Lamp2, a known lysosomal protein, and enrichment of the Plac8 protein in lysosomal fractions. The discovery that Plac8 localizes to lysosomes is consistent with prior date showing that Plac8 is enriched in the granular endosomal isolate of neutrophils, which is a modified form of lysosome. Lysosomes primarily serve as the bulk degradation centers of the cell, indicating that Plac8 has some involvement in a degradation process. These data coupled with increased Plac8 expression around necrotic centers in tumors, and increased Plac8 protein under nutrient starvation and hypoxic conditions, which has been previously shown for proteins involved in the autophagy process, suggested that Plac8 was involved in autophagosomal/lysosomal fusion. Consistent with this hypothesis, Ledford et. al. also demonstrated that neutrophils derived from Plac8 KO mice have reduced phagocytosed bacterial killing which employs the same cellular machinery as autophagosomal/lysosomal fusion.

Additionally, the tumor inhibitory effect of Plac8 KD is due to requirement of Plac8 in autophagosomal/lysosomal fusion. Plac8 KD leads to an accumulation of autophagosomes identified via electron microscopy, accumulation of the marker for autophagy LC3 and p62, and an inhibition of Lamp-2/GFP-LC3 colocalization, which are all indicative of an inhibition of autophagosomal/lysosomal fusion. Moreover DN Rab7, a known inhibitor of autophagosomal/lysosomal fusion, phenocopies Plac8 KD tumor inhibition and expression of DA Rab7 on a Plac8 KD background rescues tumor formation, as well as, LC3 and p62 accumulation and Lamp2/GFP-LC2 colocalization, indicating that Plac8 is required for autophagosomal/lysososmal fusion and this process is essential for tumor formation. These data are consistent with studies demonstrating that the pharmacological inhibitors of autophagosome fusion, chloroquinine or baflomycin A1 were effective at inhibiting tumor formation of lymphoma, colon cancer, lung cancer, and pancreatic cancer cells. Also disclosed herein is that this is specific to autophagosomal/lysosomal fusion by expression of DN Rab5a, an inhibitor of endocytosis, which share the same molecular machinery as autophagy. DN Rab5a expression slightly enhanced tumor formation in transformed YAMC cells and no effect on Capan-2 cells indicating that tumor formation specifically requires autophagosomal/lysosomal fusion and not endosomal/lysosomal fusion. Disclosed herein, for the first time, that Plac8, Rab7 activity and ultimately autophagosomal/lysosomal fusion are essential to the cancer phenotype and this is specific to autophagosomal/lysosomal fusion and not the endocytic process.

The contribution of Plac8 specifically to autophagy was demonstrated by rescuing Plac8 KD tumor inhibition, LC3 and p62 accumulation and partially Lamp2/GFP-LC3 colocalization with over-expression of the autophagy formation gene Atg12. However, over-expression of Atg12 individually also inhibited tumor formation, indicating that over-activation of autophagy is also tumor inhibitory. These two pieces of seemingly conflicting data reflect the current ideas about autophagy in cancer which suggest that autophagy can be tumor promoting and inhibitory, however, herein disclosed for the first time that this phenomena appears to be true in the same cell, suggesting that there is an optimal rate for autophagy. For the design of rational therapies to specifically modulate autophagy in cancer a determination can be made if this optimal rate for autophagy is more, less or unchanged from normal to transformed cells. The rate of autophagy can be set by oncogenic mutations, including Ras and mp53; however, oncogenic mp53 and Ras effects on autophagy have only been investigated individually. Since transformation only occurs with the cooperation of mp53 and Ras, and the resulting cancer phenotype is dependant on autophagy, understanding how oncogene cooperativity affects autophagy is of great interest.

### 3. Example 3

### Autophagy is Cooperatively Induced by Oncogenic Mutations and an Optimal Rate of Autophagy is Essential to the Cancer Phenotype

Plac8 supports autophagy and that the autophagy process is required for the cancer phenotype. The cooperative up-regulation of Plac8 in response to mutant Ras and p53 raises the question as to whether the autophagy process is induced by cooperating oncogenic mutations. Moreover for the design of rational therapies to specifically modulate autophagy in cancer a determination can be made as to whether autophagy is activated, inactivated or unchanged from normal to transformed cells. Single oncogene activation, such as PI3K and Akt1, or tumor suppressor loss, such as PTEN, DAPK1, and TSC1 or TSC2, inhibit the autophagy process, suggesting that autophagy is tumor suppressive. However, over-expression of c-myc or p53 loss or mutation has been shown to promote autophagy; suggesting that autophagy can co-exist with and can promote oncogenesis. However, the aforementioned studies have thus far only examined the effects of individual oncogenes on autophagy. Introduction of single oncogenes into normal cells can have vastly different effects on cell behavior than when introduced with a cooperating oncogene and multiple oncogenic mutations are required for cellular transformation and oncogenesis. Therefore studying autophagy in a model system where contributions from individual oncogenes and cooperating oncogenes can be ascertained reveals how autophagy activity is changed from the normal to the transformed state.

a) Autophagosomal/Lysosomal Fusion is Cooperatively Induced.

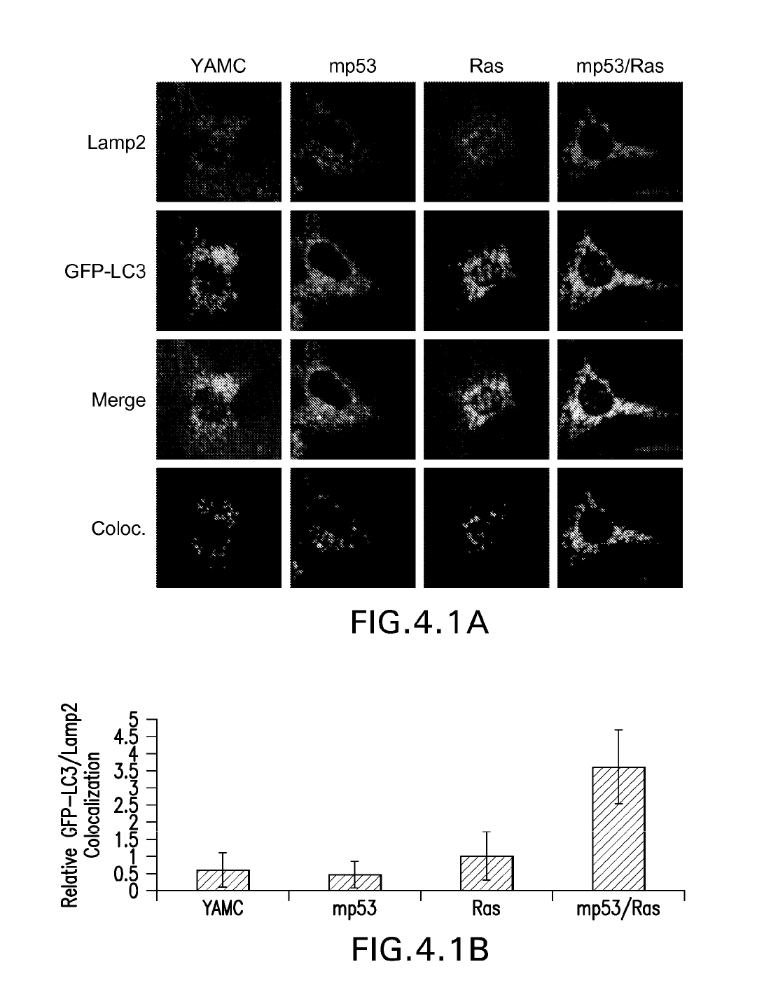

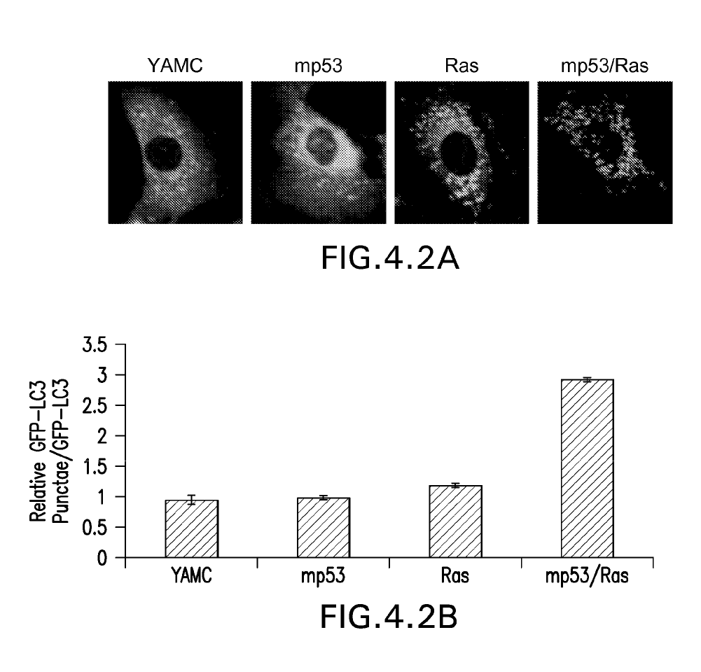

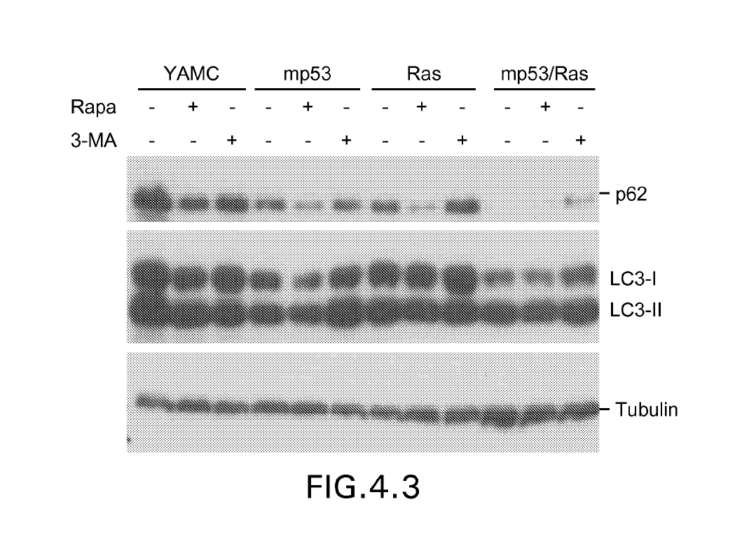

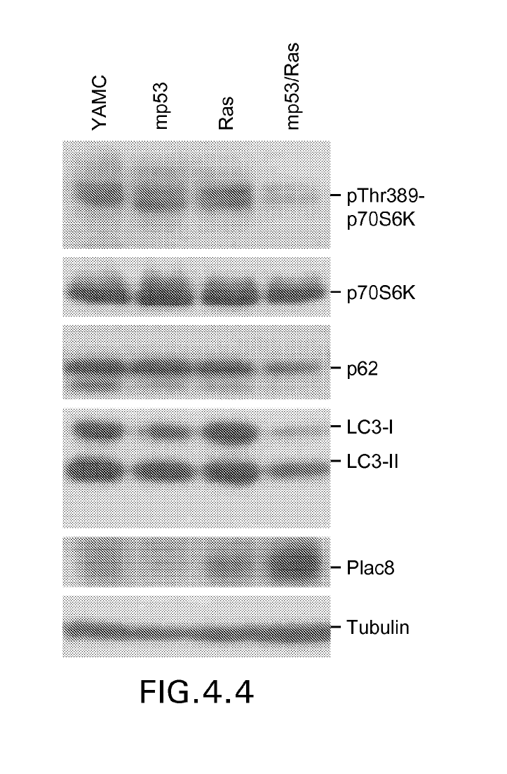

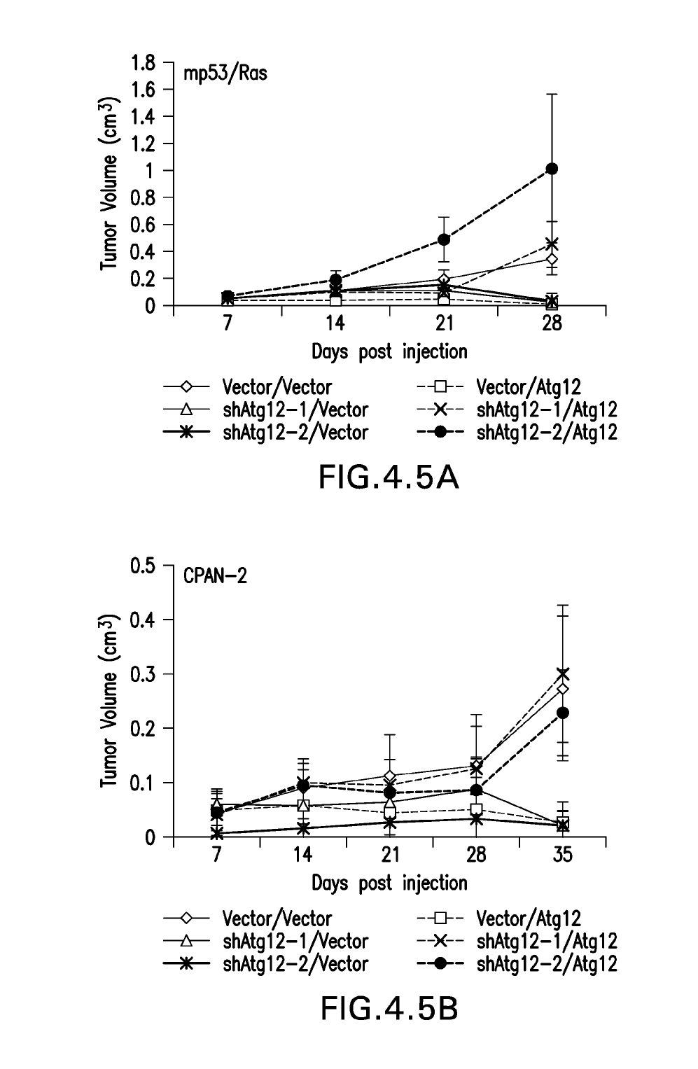

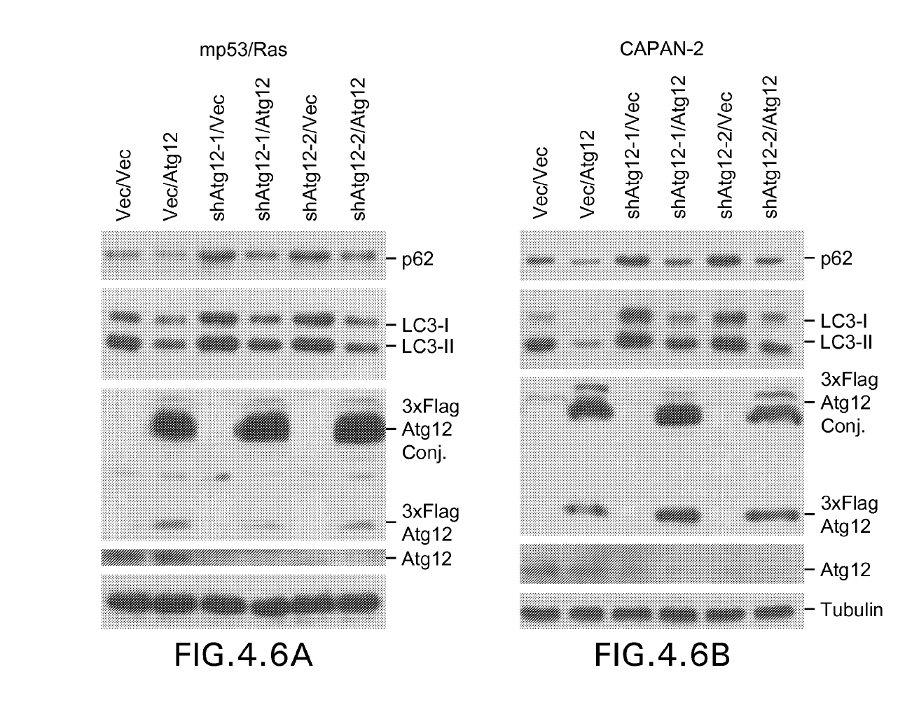

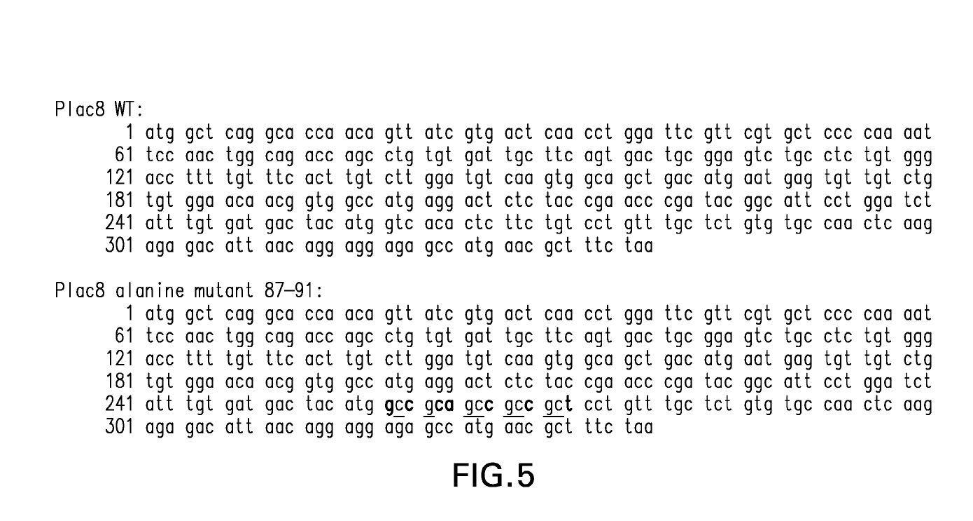

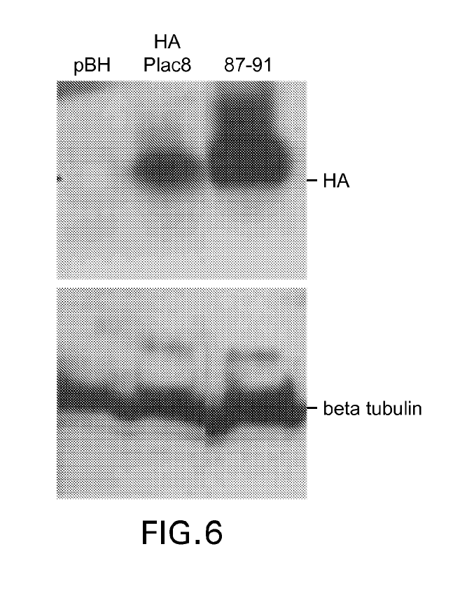

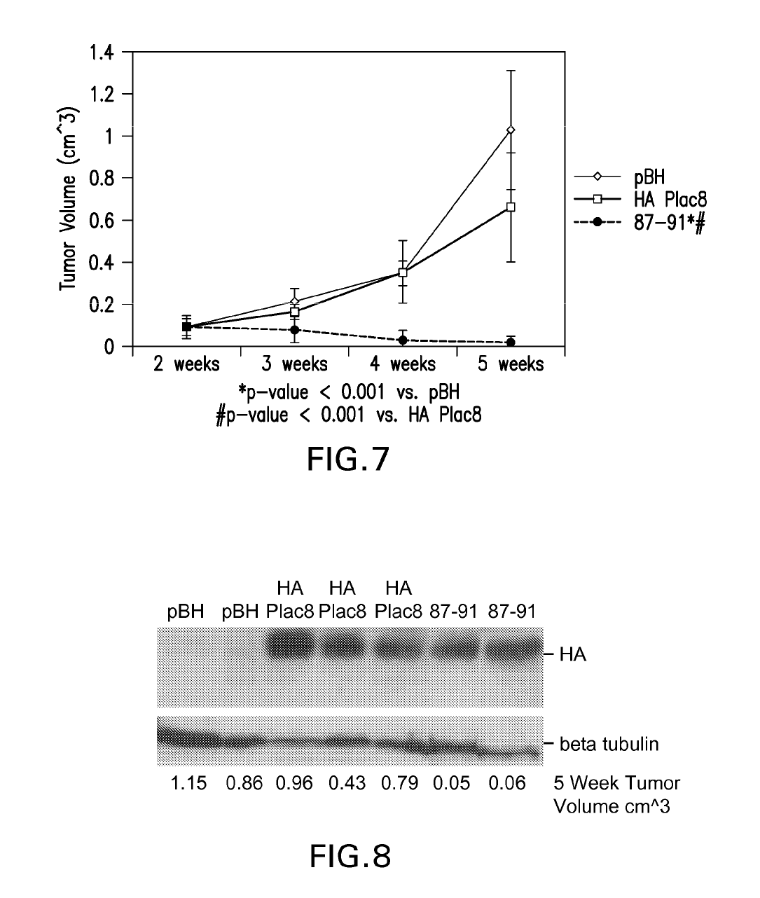

The cooperative up-regulation of Plac8 and its requirement for optimal autophagosomal/lysosomal fusion, raises the question whether the autophagosomal/lysosomal fusion process is cooperatively induced by mp53 and Ras. To examine the relative levels of autophagosomal/lysosomal fusion in normal and transformed cells GFP-LC3 was expressed via infection in YAMC cells, YAMC cells expressing mp53, RasV12, or both mutant proteins together and monitored GFP-LC3 co-localization with the lysosomal protein Lamp2. These measurements were performed under cell starvation to maximize the process of autophagosome formation (FIG. 4.1a,b). Notably, the colocalization of GFP-LC3 and Lamp2 is synergistically induced by mp53/Ras indicating that the autophagosomal/lysosmal fusion process is cooperatively induced by RasV12 and mp53.

b) Autophagosome Formation is Synergistically Induced by Cooperating Oncogenic Mutations.

The cooperative induction of autophagosomal/lysosomal fusion by mp53 and Ras demonstrates that one phase of autophagy is up-regulated. However, because the autophagy fusion process cannot proceed without the formation of autophagosomes, these data suggest that, similar to autophagosomal/lysosomal fusion, autophagosome formation is synergistically induced by cooperating oncogenic lesions. To test for this possibility, ectopic GFP-LC3 was expressed in YAMC cells, YAMC cells expressing mp53, Ras, or both mp53 and Ras together and analyzed cells for the number of emerging GFP punctae in the cells. LC3 is a cytoplasmic protein that is inserted into the membrane of the autophagosome when an autophagosome is formed (Kabeya et al., 2000) and by expressing a GFP-LC3 fusion autophagosomes can be identified by GFP-LC3 punctae. An increase in the conversion of diffuse cytoplasmic GFP-LC3 to GFPLC3 punctae indicates an increase in autophagosome formation.

The number of GFP-LC3 punctae per diffuse cytoplasmic GFP-LC3 signal was not increased by mp53, slightly increased Ras alone, and synergistically increased by mp53 and RasV12 (FIG. 4.2a, b), which indicates that autophagosome formation is cooperatively induced by Rasv12 and mp53. To further investigate cooperative autophagy induction the biochemical markers of autophagy induction, p62 degradation and conversion of LC3-I to LC3-II, in YAMC, single oncogene and mp53/Ras cells were analyzed. p62 degradation and LC3 conversion were slightly increased single oncogene cells, but further induced by cooperating oncogenes compared to YAMC cells (FIG. 4.3), indicating that autophagy is cooperatively induced by mp53 and Ras. Another possibility is that the autophagy process is not functionally inducible or limited in YAMC, mp53, and Ras. To determine if the autophagy process was still functionally inducible in YAMC, Ras, mp53 and mp53/Ras cells were treated with a pharmacological inhibitor of mTOR, thereby inducing autophagy and proteins lysates were blotted for the autophagy markers LC3 and p62 (FIG. 4.3). The data show that p62 degradation and LC3 conversion are increased in rapamycin treated YAMC, mp53 and Ras cells but are unchanged in Transformed cells. This indicates that the autophagy process is functionally inducible by mTOR deactivation in YAMC, mp53 and Ras cells, but mp53/Ras cells are insensitive to further mTOR deactivation by rapamycin.

The degradation of p62 and conversion of LC3 could also be due to another mechanism besides autophagy. To identify if the p62 degradation and LC3 conversion is specifically due to autophagy YAMC, mp53, Ras, and mp53/Ras cells were treated with 3-methyladenine, a specific pharmacologic inhibitor of autophagosome formation (FIG. 4.3). The data shows that p62 and LC3-I increase upon treatment of 3-methyadenine in mp53, Ras, and mp53/Ras cells indicating that the degradation of p62 and LC3 conversion is specific to the autophagy process. These data indicate that autophagosome formation is specifically and synergistically activated by mp53 and Ras, but only minimally or not at all by either oncogenic mutation alone.

The insensitivity only in mp53/RasV12 cells to the mTOR inhibitor rapamycin indicates that mTOR may be deactivated in these cells. The activity of mTOR can be examined indirectly via measuring the phosphorylation of p70S6K at Thr389, a specific phosphorylation substrate of mTOR. Cell extracts from YAMC, single oncogene and mp53/RasV12 cells cultured under normal growth conditions were immunoblotted for phospho-Thr389-p70S6K and total p70S6K (FIG. 4.4). The phosphorylation of p70S6K at Thr389 is synergistically down-regulated by mp53 and RasV12, while the total levels of p70S6K are equivalent, indicating that mTOR activity is specifically inhibited only when both oncogenic proteins are present. This indicates that the activation of autophagy by mp53 and Ras is consistent with synergistic deactivation of mTOR.

c) Autophagy is Required for Tumor Formation, but is Also Tumor Inhibitory if Over-Induced.

Autophagosome maturation is critical to the cancer phenotype, however, the cooperative up-regulation of autophagosome formation indicates that the formation process is critical to the cancer phenotype as well. To determine whether cooperative up-regulation of autophagy is required for tumorigenesis Atg12, a gene involved in autophagosome formation, was knocked-down using two independent constructs in mp53/RasV12 and Capan-2 cells. In addition, a genetic rescue experiment was performed by introducing a shRNA resistant form of ATG12 into these cell backgrounds to test for ATG12 specificity of the observed effects. Tumor formation was inhibited by Atg12 KD in both mp53/Ras and CAPAN-2 cell lines and was rescued by expression of the exogenous shRNA resistant Atg12 (FIG. 4.5a, 4.5c). Moreover, knock-down of Atg12 resulted in the inhibition of LC3 conversion and the accumulation of p62, indicating that autophagosome formation was inhibited (FIG. 4.6a,b) LC3 conversion and p62 degradation was restored to normal levels by expression of a shRNA-resistant form of Atg12, indicating that effects on LC3 and p62 were specific to Atg 12.

Over-expression of Atg12 led to an increase in p62 degradation and LC3-I conversion and LC3-II degradation, indicated that the whole autophagy process was further induced. This further induction of autophagy by Atg12 over-expression resulted in an inhibitory effect on tumor formation. These data indicate that both over-activating or inhibiting the autophagy process is detrimental to the cancer phenotype, and that a balance of the autophagy process appears to be essential to the cancer phenotype.

d) Summary

It is disclosed herein that the autophagy fusion process is synergistically induced by mp53 and Ras. Colocalization of GFP-LC3 labeled autophagosomes and anti-Lamp2 immunostained lysosomes was synergistically up-regulated by mp53 and Ras. This is due to, at least in part to cooperative up-regulation of Plac8. These data indicate that autophagosomal/lysosomal fusion is not only required for tumor formation, but is also activated by oncogene cooperativity, indicating that autophagosomal/lysosomal fusion is an important cooperatively induced biological process of the cancer phenotype.

Also disclosed herein is that autophagosome formation is synergistically induced by mp53 and Ras and is controlled, at least in part, by cooperative inhibition of mTOR activity. The proportion of GFP-LC3 punctae versus diffuse GFP-LC3 was synergistically up-regulated by mp53 and Ras together versus single oncogenes or vector control cells, indicating autophagosome formation is cooperatively induced. Moreover mp53 and Ras also increase degradation of p62 and conversion of LC3 over single oncogenes or vector control cells, which is insensitive to the autophagy inducing, mTOR inhibitor rapamycin, indicating that autophagy is cooperatively induced possibly by inactivation of mTOR. Furthermore phosphorylation of the mTOR target, p70-S6K, is specifically inhibited only in mp53/Ras transformed cells, indicating that mTOR kinase activity is synergistically inhibited in cells containing both mp53 and Ras. This is consistent with the data presented by Tasdemir et al. who demonstrated that loss of p53 in HT1 16 colorectal adenocarcinoma cell line, which contains an activating Ras mutation, inhibits mTOR activity and induces autophagy. Therefore, mp53 and Ras inhibit mTOR kinase activity, which contributes to the cooperative induction of autophagosome formation. These data also indicate that the whole autophagy process is induced in transformation and can be a cell protective biological process integral to the cancer phenotype.

The essential contribution of autophagosome formation to the cancer phenotype is demonstrated by specific inhibition of the autophagy process by Atg12 shRNAs and rescue with an exogenously expressed, shRNA resistant Atg12. Therefore, autophagosome formation and fusion are both cooperatively induced by mp53 and Ras, and both the autophagosome formation and fusion processes are essential to malignant cells. However, further induction of the autophagosome formation or fusion processes by Atg12 or DA Rab7, as shown herein, are also tumor inhibitory. Consistent with this is data for the autophagosome formation gene Beclin1 in malignancy, where monoallelic deletion of Beclin1 in mice was tumorigenic, however the tumors that arose still maintained one copy of the Beclin1 gene and expressed WT levels of Beclin1. These data indicate that curtailing autophagy over-induction by Beclin1 monoallelic deletion is tumor promoting, but autophagy is still maintained in the tumor by conservation of the other allele. Taken together the data and data for Beclin1 indicate there is an optimal rate of autophagy that is essential to the cancer phenotype. In conclusion further induction or inhibition of autophagy can inhibit tumor formation, however cooperative malignant transformation by mp53 and Ras specifically induces the autophagy process, indicating that inhibition of autophagy specifically targets transformed cells.

e) The Cooperatively Up-Regulated Gene Plac8 is Essential to Malignant Transformation.

The data demonstrate that synergistic up-regulation of the gene Plac8 by mutant mp53 and activated Ras is required for malignant transformation. Herein is shown that Plac8 had the largest tumor inhibitory effect upon shRNA mediated knockdown of the 24 cooperation response genes tested. Also disclosed is that neither Plac8 expression nor its knockdown by shRNA altered mp53 or Ras protein levels, indicating that the mechanism for Plac8 inhibition of tumor formation is downstream of the oncogenic mutations. Plac8 is also up-regulated in human bladder, pancreatic, ovarian, and brain cancers when compared to adjacent normal tissue, suggesting a role for Plac8 in multiple types of human cancer. For HT-29 human colorectal and CAPAN-2, Panc1 0.05, and PANC-1 human pancreatic cancer cell lines Plac8 shRNA mediated knockdown significantly inhibited tumor growth. The data thus demonstrate that synergistic regulation of the Plac8 gene by cooperating oncogenic mutations is an important feature of malignant cell transformation.

The dependence of the transformed phenotype on the initiating oncogenes, where removal of the initiating oncogenes results in the loss of the transformed phenotype has been termed “oncogene addiction”. This is suggestive of an oncogene dependent, downstream network responsible for the cancer phenotype that collapses upon removal of the initiating oncogenes. Oncogenes showing such behavior are potential targets for cancer therapeutics but are limited in number. Herein are identified non-oncogenes, such as Plac8, that function downstream of the initiating oncogenes and upon removal also result in the loss of the transformed phenotype, indicating a possible addiction to these nononcogenes. This non-oncogene addiction indicates genes responsible for the downstream effects of oncogenic mutations, and thus increase the number of potential intervention targets beyond mutated oncogenes.

f) Plac8 is Required for Autophagosomal/Lysosomal Fusion.

Specifically the autophagosomal/lysosomal fusion is the process underlying malignant transformation for which Plac8 is required. The inhibition of autophagosomal/lysosomal fusion has been shown to result in an accumulation of autophagosomes, autophagosomal markers p62 and LC3, and a decrease in colocalization of the lysosomal marker Lamp2 and the autophagosome marker GFP-LC3. Indeed upon loss-of function experiments by Plac8 shRNA mediated knock down (KD) or expression of dominant-negative (DN) Rab7, a gene required for autophagosomal lysosomal fusion an accumulation of p62 and LC3 proteins, an accumulation of autophagosomes by electron microscopic analysis of Plac8 KD cells versus vector control, and a decrease in Lamp2/GFP-LC3 colocalization in both Plac8 KD and DN Rab7 expressing cells were demonstrated. Furthermore it is disclosed herein that Plac8 KD and DN Rab7 inhibit tumor formation, and conversely that rescuing the rate of autophagosomal/lysosomal fusion in Plac8 KD cells by expression of constitutively activated Rab7 restores p62 and LC3 levels, Lamp2/GFP-LC3 colocalization and the ability of the cells to form tumors. Autophagosomal/lysosomal fusion is essential for the transformed state. In support of this idea, the data further indicates that the malignant state is indeed specifically dependent on the autophagy process, as inhibiting endocytosis by preventing fusion of endosomes with lysosomes via expression of DN Rab5a does not inhibit tumorigenicity.

Additional support for the conclusion that tumor formation specifically requires autophagosomal/lysosomal fusion came from experiments designed to increase the rate of autophagy by over-expressing Atg12 in Plac8 KD cells. This restores p62 and LC3 levels, restores Lamp2/GFP-LC3 colocalization and rescues tumor formation. Consistent with the work inhibition of autophagosomal/lysosomal fusion by pharmacologic alkylinization of the lysosomal lumen by Cholorquine or Bafilomycin A1 also inhibits tumor growth, although the alkalinizing action of Chloroquine and Bafilomycin A1 on lysosomes is not specific to autophagosomal/lysosomal fusion in cancer cells and has many other effects on lysosomal function in normal cells. Conversely, the synergistically up-regulated nature of Plac8 indicates that Plac8 up-regulation is a specific regulation point for autophagosomal/lysosomal fusion in malignant cells.

Inhibiting other lysosomal proteins shown to be involved in the autophagy fusion process such as Lamp2 and Rab7 are also therapeutic targets, however many lysosomal protein loss-of-function mutations in humans are associated with overt disease phenotypes. Lamp2 truncation mutations results in Danon's Disease in humans, a glycogen storage disorder associated with hypertrophic cardiomyopathy and skeletal muscle weakness, with a similar phenotype in Lamp2 knock out mice. Rab7 loss-of-function mutations in humans have been linked to the ulcerating peripheral neuropathy Charcot-Marie-Tooth syndrome type 2B. Inhibiting the function of these proteins may result in drastic side effects similar to the genetic disease phenotypes. Plac8 may be an exception as the Plac8 knock out mouse is viable with no overt phenotype and mutations in Plac8 have not been linked to human disease, however Plac8 is still important for the malignant phenotype. Why Plac8 is an exception is unknown, however, it may be possible that since Plac8 only partially co-localizes with Lamp2, the Plac8 positive/Lamp2 negative vesicles may represent a sub-class of lysosomes that are distinctly required in the autophagy process in malignant cells, however further testing is need to examine this possibility.

g) Autophagosome Formation and Fusion to Lysosomes are Cooperatively Induced by mp53 and Ras.

The role of the cooperatively up-regulated gene Plac8 in autophagosomal/lysosomal fusion led to investigating whether the autophagosomal/lysosomal fusion process is cooperatively up-regulated in transformed cells. Indeed, Lamp2/GFP-LC3 colocalization is synergistically up-regulated by mp53 and Ras indicating that the autophagosomal/lysosomal fusion process is also cooperatively up-regulated by mp53 and Ras. Similarly, autophagosome formation is induced only in the presence of both mp53 and activated Ras, presumably via reduction of mTOR activity, an inhibitor of autophagosome formation. mTOR deactivation in the presence of p53 deactivation and Ras activation is consistent with data from Tasdemir et. al., who demonstrated that p53 deactivation in HCT116 cells, which contain a Ras activating mutation, resulted in mTOR deactivation and increased autophagosome formation, however, the individual contributions of the oncogenic mutations were not ascertained. The laboratory has previously demonstrated that oncogenic Ras/Raf activation can have simultaneously opposing or even completely opposite signaling effects that change upon introduction of p53 inactivation, which contribute to the unrestricted proliferation and invasive altered cell biology required for the cancer phenotype. Previous studies have indicated that oncogenic Ras can induce autophagy through MAPK signaling and inhibit autophagy through PI3K signaling, which activates mTOR.

h) Autophagic Balance is Essential to Malignant Transformation.

Inhibition of autophagosomal/lysosomal fusion through Plac8 shRNA mediated KD or expression of DN Rab7, or inhibition of autophagosome formation through Atg12 KD inhibit tumor formation indicating that autophagy is essential to malignant transformation. Autophagy has been generally described as a catabolic process, involved in the degradation of cellular components. This appears counter-intuitive in the face of rapid proliferation and anabolic metabolism in the cancer cell. However, in light of the fact that autophagy specifically degrades damaged proteins and organelles, autophagy can be described as a bulk cellular recycling mechanism in which metabolites are released for anabolism that are otherwise trapped within non-functional cellular components. Indeed it has been demonstrated that induction of autophagy via loss of p53 confers a resistance to ATP depletion and cell death induced by metabolic stress, which is lost when autophagy is inhibited. Oxygen and nutrient supply is often low in malignant tumors due to the rapid growth and low vascularization, and cancer cells also do not efficiently convert metabolites to energy because of a reduction of oxidative phosphorylation. Therefore, an increase in cellular recycling in the face of decreased nutrients and increased proliferation makes efficient use of what metabolites are available, thus increasing the fitness of malignant cells. Conversely, over-activation of autophagy can lead to cellular self-cannibalism, resulting in destruction of functional components and decreasing the fitness of malignant cells. Indeed, over-activation of autophagy either by expression of DA Rab7 or over-expression of Atg12 also inhibits tumor formation. Furthermore, re-setting autophagy back to baseline levels in Atg12 over-expressing cells by either Plac8 KD, expression of DA Rab7 or Atg12 knock down, as demonstrated by re-adjustment of LC3 conversion and p62 degradation back to vector control levels, re-establishes tumor formation capacity. Together these data indicate that there is an optimal level of autophagy activity that is essential to the cancer cell. Thus, either inducing or inhibiting autophagy inhibits cancer growth. However, because cooperative transformation induces autophagy, suppression of autophagy yields cancer cell specificity for intervention, as a cancer specific autophagy gene, Plac8, was identified which is an ideal intervention target.

### 4. Example 4

### Cell Culture

YAMC, mp53, Ras, mp53/Ras cells were maintained at 33 C in water-jacketed humidified incubators with 5% CO₂ in RPMI (Gibco) medium supplemented with 10% (v/v) fetal bovine serum (FBS) (Hyclone), 2.5 ug/mL gentamicin (Gibco), 1× insulin-selenium-transferrin-A (ITS-A) (Gibco) and 25 U/ml interferon—(R&D Systems). Derivative mp53/Ras cells infected with pSuper.retro, pBabe, FG12/FUG12, pLKO.1, and/or pLenti6/Ubc/V5 constructs were maintained at 39 C in RPMI medium supplemented with 10% (v/v) FBS, 2.5 μg/mL gentamicin, and 1×ITS-A. All YAMC, Vector, mp53, Ras, mp53/Ras and derivative mp53/Ras cells were cultured on 1 μg/cm² collagen I-coated (BD Biosciences). HT-29 and PANC-1 cell lines were maintained at 37 C in a humidified water-jacketed incubator with 5% CO₂ in DMEM (Gibco) containing 10% FBS, 100 g/ml kanamycin (Sigma) and 2 μg/mL gentamicin. CAPAN-2 and Panc1 0.05 cell lines were maintained at 37 C in a humidified water-jacketed incubator with 5% CO₂ in RPMI containing 10% FBS, 100 g/ml kanamycin and 2 g/mL gentamicin. Ecotropic phoenix, amphitropic phoenix, and 293TN viral producer cells were maintained at 37 C in a humidified water-jacketed incubator with 5% CO₂ in DMEM containing 10% FBS, 100 g/ml kanamycin (Sigma) and 2 g/mL gentamicin.

### 5. Example 5

### Retrovirus-Mediated Gene Transfer

Prior to transfection for viral production of ecotropic phoenix cells (murine cell specific) were selected for two weeks with 400 ug/mL Hygromycin (Invitrogen) and 1 ug/ml diphtheria toxin (Sigma) for two weeks prior to use, then passaging for another week without selective antibiotics keeping the cells cultures below approximately 70% confluency. For amphitropic phoenix cells the same procedure was followed except omitting the diphtheria toxin selection. Approximately 6 hours before transfection cells were seeded at a density of 2.5×10⁵ cells per 10 cm dish in 10 ml of DMEM media. Replication-deficient murine infectious retroviruses were generated by transiently transfecting ecotropic phoenix producer cells with 20 g of DNA (in 5001 of ddH2O including 62.51 of 2M CaCl₂) by standard calcium phosphate precipitation using 5001 of 2×HBS (280 mM NaCl, 10 mM KCl, 1.5 mM Na₂HPO₄ 2H2O, 12 mM dextrose, 50 mM HEPES, pH 7.05) overnight in 10 ml of DMEM containing 10% FBS, 1 00 g/ml kanamycin and 2 g/mL gentamicin. The 2×HBS was added drop-wise to water/DNA/CaCl₂ mixture with vortexing and then added to phoenix cell media after incubating the precipitation reactions for 15 minutes at room temperature. Replication-deficient human infectious retroviruses were generate by transiently transfecting amphitropic phoenix producer cells with 10 g of vector DNA and 10 g of VSVG vector DNA (in 5001 of ddH2O including 62.51 of 2M CaCl₂) by standard calcium phosphate precipitation using 5001 of 2×HBS overnight in 10 ml of DMEM containing 10% FBS, 100 g/ml kanamycin and 2 g/mL gentamicin. After overnight transfection the media of the phoenix cells was removed and replaced with 4 ml of DMEM containing 10% FBS, 100 g/ml kanamycin and 2 g/mL gentamicin. Twenty-four hours before infection target mp53/Ras cells were plated at 2.5×10⁵ onto collagen-I coated 10 cm dishes and HT-29 target cells at 7.5×10⁵ were plated on 10 cm dishes. Approximately 24 hours later viral supernatants from phoenix cells were collected, filtered through 0.45 um syringe filters (Pall), overlaid onto target cells, and polybrene (sigma) was added to a 8 ug/ml final concentration to facilitate infection efficiency. After 90 minutes, the infectious media was removed from the target cells and another fresh viral supernatant was overlaid onto the target cells with polybrene. This was repeated 10 more times for pSUPER.retro vectors and 4 more times for pBabe vectors. After completion of the last infection, supernatants were removed off of the target cells and 10 ml of fresh maintenance media was added. Target cells were allowed to proliferate for approximately two days and then were trypsinized and plated into maintenance media containing either 5 ug/ml puromycin for puromycin resistant vectors and 250 ug/ml of hygromycin for hygromycin resistant vectors, or both for cells infected with both antibiotic resistant vectors.

### 6. Example 6

### Lentivirus-Mediated Gene Transfer

Approximately 6 hours prior to tranfection 3×10⁶ 293TN cells were plated per 10 cm dish in 6 ml of DMEM containing 10% FBS, 100 g/ml kanamycin and 2 g/mL gentamicin. Replication-deficient human infection lentiviruses were generated by transfecting 239TN lentiviral producer cells with 2 ug of lentiviral vector, 1.5 ug of VSV-G vector and 3 ug of Pax2 packaging vector DNA were mixed in 600 uL of PBS and 18 uL of Fugene-HD (Roche) was added and mixed by tapping the tube. Transfection mixture was incubated for 30 minutes at room temperature then added drop wise to 293TN cell media. After overnight transfection the media of the 293TN cells was removed and replaced with 5 ml of DMEM containing 10% FBS, 100 g/ml kanamycin and 2 g/mL gentamicin. Twenty-four hours before infection target CAPAN-2, PANC-1 and Panc 10.05 cells were plated at 1×10⁶ cells per 10 cm dish. Approximately 24 hours later viral supernatants from 293TN cells were collected, filtered through 0.45 um syringe filters (Pall), overlaid onto target cells, and polybrene was added to a 8 ug/ml final concentration to facilitate infection efficiency. After approximately 4 hours, the infectious media was removed from the target cells and another fresh viral supernatant was overlaid onto the target cells with polybrene. This was repeated one more time for FUG12 and pLKO.1 vectors, for a total of three infections and four more times for pLenti6/Ubc/V5 vectors. After completion of the last infection, supernatants were removed off of the target cells and 10 ml of fresh maintenance media was added. Target cells were allowed to proliferate for approximately two days, then were trypsinized and plated into maintenance media containing either 5 ug/ml puromycin for puromycin resistant vectors and 5 ug/ml of blasticidin for blasticidin resistant vectors, or both for cells infected with both antibiotic resistant vectors.

### 7. Example 7

### Immunocompromised Mouse Tumorigenicity Assay

Cells were cultured on 15 cm dishes for two days without selective antibiotics at 39 C for mp53/Ras cells and at 37 C for HT-29, CAPAN-2, PANC-1, and 5 Panc10.05. Cells were plated at the following densities: mp53/Ras—7.5×10⁵, HT-29 —1.5×10⁶, CAPAN-2 —4×10⁶, PANC—4×10⁶, and PANC 10.05—2×10⁶. Cells were trypsinized, counted and re-suspended in RPMI medium lacking supplements for mp53/Ras, CAPAN-2, and Panc10.05, and DMEM lacking supplements for HT-29 and PANC-1 at a density of 5×10⁵/100 ul for mp53/Ras and CAPAN-2 cells, 1.25×10⁵/100 ul for HT-29 cells, 5×10⁶/100 ul for PANC-1, 2×10⁶/100 ul for Panc10.05. Matrigel (BD biosciences) was mixed with PANC-1 and Panc10.05 cell/medium mixture at a 1:1 ratio. Cell mixtures were then injected intra-dermally, bilaterally into the flanks of CD1-Foxn1 nude mice (Uracell) 5×10⁵-2000-143 (1)) in the following numbers: mp53/Ras cells, HT29 1.25×10⁵ cells, 5×10⁵ CAPAN-2-cells. Cell mixtures were injected intradermally, bilaterally into the flanks of NOD/SCID (UCAR-20060-166 (1)) mice in the following cell numbers per injection: PANC-1 —2.5×10⁶ cells and Panc10.05 —1×10⁶ cells. Tumor volume was measured every week for 4 weeks for mp53/Ras and HT-29 cells, 5 weeks for CAPAN-2 cells, and 6 weeks for PANC-1.

### 8. Example 8

### Quantitative RT-PCR

YAMC, vector, mp53, Ras, and mp53/Ras cells were cultured for two days at 39 C in RPMI with 10% FBS medium and gentamicin in collagen-I coated 10 cm dishes. Cells were then washed twice with PBS and cultured for an additional day in RPMI with gentamicin at 39 C. HT-29, CAPAN-2, PANC-1 and Panc10.05 cells were cultured for two days at 37 C in maintenance media without selective antibiotics. Cells were plated at the following densities: YAMC—3×10⁵, mp53 —2.75×10⁵, Ras and mp53/Ras—2.5×10⁵, HT-29, CAPAN-2, PANC-1 and 5 Panc 10.05 —7.5×10⁵ cells per 10 cm dish. Cells were trypsinized, pelleted at 1,200 rpm for 3 minutes at 4 C, snap-frozen in liquid Nand stored at −80 C.

Total RNA was extracted using Qiashredder and RNAeasy Mini RNA extraction kits (Qiagen). Five ug of total RNA was used for reverse transcription reactions. The RNA was first mixed with 10 ul 5× first strand buffer, 5 ul 0.1M dithothrietol, 5 ul 10 pm/ul random hexamers (Invitrogen) and 2 ul 10 mM dNTPs (Invitrogen) and denatured for 5 minutes at 85 C. Reverse transcription reactions were then incubated for 2 minutes on ice and 1 ul of RNaseOUT (Invitrogen) and 1 ul of Single Strand II reverse transcriptase (Invitrogen) were added to each reaction. Reverse transcription reactions were then incubated at 42 C for one hour. Quantitative PCR reactions were prepared in triplicate using (per reaction) 1 ul cDNA, 12.5 ul SYBR Green (BioRad), 5 ul 1 pmol/ul forward and reverse qPCR primers, and 6.5 ul of ddH2O. All primer sets used an annealing temperature of 58 C and 40 cycles. PCR reactions were run on an iCycler (BioRad). Fluorescence intensity values were analyzed byCt method to generate relative fold expression values normalized to RhoA for murine and GAPDH for human samples and then to YAMC or vector-infected control samples.

### 9. Example 9

### Western Blotting

YAMC, vector, mp53, Ras, and mp53/Ras cells were cultured for two days at 39 C in RPMI with 10% FBS medium and gentamicin in collagen-I coated 15 cm dishes. HT-29, CAPAN-2, PANC-1 and Panc 10.05 cells were cultured for two days at 37 C in maintenance media without selective antibiotics on 15 cm dishes. For protein extraction cells were washed twice with ice cold phosphate buffered saline, carefully scraped off plate into ice cold PBS, pelleted at 1200 rpm for 3 minutes at 4 C, and lysed in an equal volume of ice cold RIPA buffer (50 mM TrisHCl (pH 7.5), 150 mM NaCl, 1% Nonidet P-40 (IGEPAL), 0.5% sodium deoxycholate, 0.1% sodium dodecyl sulfate) supplemented with a protease inhibitor cocktail (Roche Diagnostics), 1 uM phenylmethanesulfonyl fluoride (Sigma) and 50 uM NaF (Sigma). The cell lysates were transferred to 1.5 mL eppendorf tubes and incubated at 4 C for 30 minutes with mixing. Lysates were spun down at 10,000 g for 10 minutes and the supernatants were transferred to new 1.5 ml eppendorf tubes. Protein concentration was quantified using Bradford reagent (BioRad) and a Genesys 1 0UV spectrophotometer (Spectronic Unicam). The appropriate volume of 5×SDS Loading buffer was then added (1.5M TrisHCl (pH 6.8), 50% glycerol, 10% sodium dodecyl sulfate, 25%-mercaptoethanol, and 0.125% bromophenol blue), samples were boiled for five minutes on ice, and then incubated on ice. Proteins from cell lysates were separated by 10 or 15% SDS-PAGE and semi-dry transferred to PVDF membranes (Millipore). Membrane was blocked with phosphate buffer saline-0.1% Tween 20 (PBST) with 5% milk for 1 hr. at room temperature. Proteins were detected with primary antibodies at the following dilutions in PBST with 5% milk at 4 C overnight: HA-tagged Plac8 —1:1000 dilution of HA antibody (Roche), —Tubulin—1:1000 of—Tubulin antibody (Santa Cruz), Plac8 —1:500 of Plac8 antibody (PRF&L, Inc.), Lamp2 —1:5000 of Lamp2 antibody (Abcam), Rab7 —1:1000 Rab7 antibody (Sigma), LC3 —1:2000 of LC3B antibody (Sigma), RhoA—1:1000 RhoA antibody (Santa Cruz), Cathepsin D—1:1000 Cathepsin D antibody (Santa Cruz), p62 —1:5000 of p62 antibody (PROGEN), 3× Flag-tagged proteins—1:1000 dilution of 3× Flag antibody and 1:2500 of HRP-conjugated 3× Flag antibody (Sigma), Rab5a—1:1000 dilution of Rab5 antibody (Abcam), phosphop70S6K (Thr389)—1:1000 dilution of phospho-p70S6K (Thr389) antibody (Cell Signaling), p70S6K—1:1000 dilution of p70S6K antibody (Cell Signaling), Atg12—1:250 Atg12 antibody (Sigma). After overnight primary antibody incubation, membranes were washed three times for 10 minutes each with PBST at room temperature. Membranes with primary antibodies that were not HRP-conjugated were then incubated with the appropriate secondary HRP-conjugated antibody at a 1:5000 dilution in PBST with 5% milk for 1 hr. at room temperature. Membranes were then washed three times for 10 minutes each with PBST at room temperature, and developed with ECL plus (GE Healthcare) for chemiluminescent protein detection.

### 10. Example 10

### Immunofluorescent Staining of Cells

Cells were plated onto collagen-1 coated 22 mm glass coverslips (BD Biosciences) in 6 well plates at 5×10⁴ cells per well. The cells were allowed to proliferate for two days in the appropriate maintenance media at 39 C for mp53/Ras cells and 37 C for human cancer cells lines. The plated cells were then washed twice with PBS and 100% methanol at −20 C was carefully overlayed onto the cells and incubated at −20 C for five minutes. The methanol was removed, the cells were washed twice with PBS, and then blocked with PBS with 5% goat serum and 0.1% Triton-X at 37 C for one hour. After one hour the blocking solution was removed and the primary antibody(s) was added in the appropriate dilution in PBS with 5% goat serum and 0.1% Triton-X as follows: Plac8 —1:50 Plac8 antibody (PRF&L), Lamp2 —1:100 Lamp2 antibody (Abcam). The cells were incubated with primary antibody overnight at 4 C with shaking. The next day cells were washed 3× for 10 minutes with PBS, then stained with the appropriate secondary antibody at a 1:100 dilution in PBS with 5% goat serum and 0.1% Triton-X for 1 hr. at room temperature in the dark. The immunostained cells were then washed 3× for 10 minutes with PBS, and mounted in VectaSheild Mounting Media (Vector Labs). Cells were analyzed and imaged using a Leica inverted confocal microscope (Lieca).

### 11. Example 11

### Subcellular Fractionation and Lysosome Isolation

mp53/Ras cells were plated at 7.5×10⁵ cells on 15 collagen-I coated 15 cm dishes and allowed to proliferate for two days at 39 C in RPMI medium supplemented with 10% (v/v) FBS, 2.5 ug/mL gentamicin. The cells were then washed twice with ice cold PBS, carefully scraped off into cold PBS and then pelleted at 1,200 rpm for 5 minutes at 4 C. A small fraction of the cells was retained and lysed in RIPA buffer for the whole cell lysate (WC) sample. Lysosomes were then isolated using a Lysosomal Isolation Kit (Sigma). In short the cell pellet was resuspended in 2.7 volumes of 1× extraction buffer, then homogenized in a Dounce homogenizer for 25 strokes on ice. The homogenized sample was then centrifuged at 1000×g for 10 minutes. The pellet was saved as the nuclear fraction (N) sample. The supernatant was then centrifuged at 20,000×g for 20 minutes. The supernatant was removed and saved as the cystosolic fraction (C) sample, and the crude lysosomal fraction pellet was resuspended in a minimal volume of 1× extraction buffer. A small aliquot was saved for the crude lysosomal fraction (CL) sample. To isolate lysosomes from other organelles 505 ul of Optiprep and 275 ul of Optiprep dilution buffer were added per 800 ul of resuspended crude lysosomal fraction. CaCladded to final concentration of 2 was 8 mM, the solution was mixed, and then incubated on ice for 15 minutes. The solution was centrifuged at 5000×g for 10 minutes at 4 C. The supernatant was removed and saved as the purified lysosomal fraction (L) sample and the pellet was saved as the microsomal pellet (M) sample. The pellet samples were resuspended in RIPA buffer, the concentration of protein in all the fraction samples was quantified by Bradford reagent, SDS sample buffer was added to all samples, and the samples were boiled as described in the Western Blotting section. Proteins in each fraction were separated and immunoblotted as described in the Western Blotting section.

### 12. Example 12

### Proteinase-K (PK) Treatment of Lysosomes

The crude lysosmal fraction was isolated as described in the subcelluar fractionation and lysosome isolation section and resuspended in 50 mM Tris buffer, pH 7.4. The total protein was quantified as described in the western blotting section. The crude lysosomal fraction was aliquoted into three 1.5 ml eppendorf tubes containing 10 ug of protein. One sample was incubated at 37 C for 30 minutes, 0.5 ug/ml of PK was added to another sample and incubated at 37 C for 30 minutes, 0.5 ug·ml of PK and 1.0% Triton-X was added to the last sample and incubated at 37 C for 30 minutes. After incubation samples were placed on ice and 1 mM of PMSF was added to quench PK activity. SDS sample buffer was added, the samples were boiled, and the samples were analyzed by western blotting as described in the western blotting section.

### 13. Example 13

### Tumor Sectioning and Immunofluorescent Staining

GFP expressing mp53/Ras cells were FAC sorted for GFP expression and injected into CD1-Foxn1 nude mice as described in the immunocompromised mouse tumorigenicity assay section. Tumors were then dissected from mice after 4 weeks and embedded in CryoMount cryogenic mounting media (Triangle Biomedical Sciences) at −20 C. The mounted tumors were then cryosectioned to 40 um sections and mounted on slides. To fix the tumor tissue the slides were dipped into 100%, −20 C methanol for 5 minutes, then washed 3× with PBS. Tumor sections were blocked with PBS with 5% goat serum and 0.1% Triton-X for 1 hr. at 37 C in a humidification chamber, the blocking solution was removed, then the primary Plac8 antibody was added at a 1:50 dilution in PBS with 5% goat serum and 0.1% Triton-X, and the sections were incubated overnight at 4 C in a humidification chamber. The next day the tumor sections were washed 3× for 10 minutes with PBS and the secondary anti-Rabbit-Alexa546 antibody was added at a 1:100 dilution in PBS with 5% goat serum and 0.1% Triton-X with a 1:5000 dilution of Topro3 Iodide (Molecular Probes) for 1 hr at 37 C in a humidification chamber. The tumor sections were then washed 3× for 10 minutes each in the dark, overlaid with VectaSheild Mounting Media (Vector Labs), and a glass coverslip placed overtop. Tumor sections were analyzed and imaged using a Leica inverted confocal microscope.

### 14. Example 14

### Quantification of GFP-LC3 Punctae Formation and Lamp2/GFP-L C3 Colocalization

GFP-LC3 expressing cells were fixed, stained, mounted, and imaged by the methods described in previous section titled Immunofluorescent staining of cells. To quantify GFP-LC3 punctae images were analyzed using the ImageJ plug-in Watershed Segmentation. The image produced by selecting Object/Background binary was inverted and overlaid on top of the GFP-LC3 image. The resulting image was quantified by measuring the mean green and blue signals per image and dividing the blue signal by the total green signal to get the amount of punctae per total GFP-LC3 expressed in the cell. This ratio was then normalized to the mean YAMC ratio of punctae formation. To quantify GFP-LC3 colocalization images were analyzed using the ImageJ plug-in Colocalization Finder. The images produced highlight colocalization from the red channel (Lamp2) and green channel (GFP-LC3) in white. These images are then merged and the mean green signal and blue signal (quantifies white signal) are quantified per image. The mean blue signal is then divided by the green signal to derive a ratio of colocalization per green (GFP-LC3) signal. These ratios are then normalized to the mean vector control or YAMC ratio.

### 15. Example 15

### Endocytosis of Fluorescently Labeled Dextran

Cells were plated at 2.5×10⁵ cells for vector and Rab5a DN mp53/Ras cells on collagen-I coated 10 cm dishes, and 7.5×10⁵ cells for vector and Rab5a DN CAPAN-2 cells on 10 cm dishes and allowed to proliferate for two days in maintenance media. Cells were then treated with 50 uM of Alexa-488 labeled 10,000 MW dextran (Invitrogen) for 1 hr in maintenance medium. The cells were then washed 3× with PBS and trypsinized. Cells were pelleted, resuspended in PBS with 1% BSA, transferred to FACS tubes (Falcon), and placed on ice. DAPI was added to a final concentration of 1 mM and cells were FACS analyzed for Alexa-488 signal, with DAPI exclusion of dead cells. To image cells mp53/Ras and CAPAN-2 cells infected with vector control virus or stabily expressing Rab5a DN were plated onto glass coverslips as described in the immunofluorescent staining of cells section. Cells were then treated with 50 uM of Alexa-488 labeled 10,000 MW dextran for 1 hr in maintenance medium. The media containing dextran was removed and the cells were washed 3 times with PBS. The cells were then fixed with 4% paraformaldehyde, stained with Topro3 iodide, mounted on slides and imaged as described in the immunofluorescent staining of cells section.

### G. SEQUENCES

Linkers and Targets for pSUPER.retro Used in this Thesis
pSUPER.retro-Neutral Linker:

Forward-GATCCCCAGGCAGTGCGATCCTCCGTTTCAAGAGATATCCG
GTAATCTCCAAATTTTTTGGAAA
Reverse-AGCTTTTCCAAAAAATTTGGAGATTACCGGATATCTCTTGA
AACGGAGGATCGCACTGCCTGGG

shRNA Target Sequences:

Murine Plac8 155- CTGGCAGACCAGCCTGTGT
Murine Plac8 240 - GTGGCAGCTGACATGAATG
Murine Plac8 461 - GCTCAACTCAGCACACACT
Human Plac8 259 - GTTGCAGCTGATATGAATG
Human Plac8 464 - GCTCTTACCGAAGCAACAA
Murine Atg12 705 - GGAGACTGAAGTTGTATGT
Murine Atg12 1860 - GCAGACTGAAAGTTTAAGA
Human Atg12 G-9 - CGAATGTAATGTGAATGGAAT
Human At12 G-12 - TGTTGCAGCTTCCTACTTCAA
Real-Time PCR primers used in this thesis
Murine Plac8 Forward -D GCTCAGGCACCAACAGTTATC
Murine Plac8 Reverse -D GCTGCCACTTGACATCCAAG
Human Plac8 Forward -D GTGCCTTGGGTGTCAAGTTG
Human Plac8 Reverse -D CCAGGGATGCCATATCGGG
Murine RhoA Forward -D AGCTTGTGGTAAGACATGCTTG
Murine RhoA Reverse -D GTGTCCCATAAAGCCAACTCTAC
Human GAPDH Forward - ACCACAGTCCATGCCATCAC
Human GAPDH Reverse -D TCCACCACCCTGTTGCTGTA
Cloning primers used in this thesis
Murine Plac8 Forward -D CGCGGATCCACCATGGCTCAGGCACCAAC
Murine Plac8 Reverse -D CGCGTCGACCTAGAAAGCGTTCATGGCTCTCCT
LC3B Cloning Forward -D CGCAGATCTACCATGCCGTCCGAGAAGAC
LC3B Cloning Reverse -D CGCGTCGACCTACACAGCCATTGCTGTCCCG
GFP-LC3 Cloning Forward - CGCACCGGTCCACCATGGTGAGCAAGGG
GFP-LC3 Cloning Reverse -D CGCCGTACGTACACAGCCATTGCTGTCCCG
Rab7 Cloning Forward -DCGCTGATCAACCATGACCTCTAGGAAGAAAGTGTTG
Rab7 Cloning Reverse -D CGCGTCGACCTAACAACTGCAGCTTTCTGC
Atg12 Cloning Forward - CGCGGATCCACCATGTCGGAAGATTCAGAGGTTGT
Atg12 Cloning Reverse - CGCGTCGACCTATCCCCATGCCTGGGATTT
3xFlag F - CGGACTAGTCCACCATGGACTACAAAGACCATG
Site directed mutagenesis primers used in this thesis
Plac8 240 Forward - GTCTTGGATGTCAGGTCGCCGCGGACATGAACGAGTGTTGTCTGTG
Plac8 240 Reverse - CACAGACAACACTCGTTCATGTCCGCGGCGACCTGACATCCAAGAC
Rab7 T22N Forward - CTCTGGTGTTGGAAAGAACTCTCTCATGAACCAGTA
Rab7 T22N Reverse - TACTGGTTCATGAGAGAGTTCTTTCCAACACCAGAG
Rab7 Q67L Forward - GGACACAGCCGGTCTAGAACGGTTCCAG
Rab7 Q67L Reverse - CTGGAACCGTTCTAGACCGGCTGTGTCC
Rab5a S34N Forward - GGAGAGTCTGCTGTTGGCAAAAACAGCCTGGTTCTTCGCTTTGTG
Rab5a S34N Reverse - CACAAAGCGAAGAACCAGGCTGTTTTTGCCAACAGCAGACTCTCC
Linkers for pBabe N-Terminal Tag Vectors
3xFlag Tag Forward - GATCACCATGGACTACAAAGACCATGACGGTGATTAT
AAAGATCATGACATCGACTACAAGGATGACGATGACA AGGGATCCAGCACA
3xFlag Tag Reverse - CTGGATCCCTTGTCATCGTCATCCTTGTAGTCGATGT
CATGATCTTTATAATCACCGTCATGGTCTTTGTAGTCC ATGGT
HA Tag Forward - GATCACCATGGGATACCCATACGACGTCCCAGATTACGCCACTGGAG
HA Tag Reverse - GATCACCATGGGATACCCATACGACGTCCCAGATTACGCCACTGGAG

### H. REFERENCES

Abate-Shen, C., and Shen, M. M. (2000). Genes Dev 14, 2410-34. Amaravadi, R. K., Yu, D., Lum, J. J., Bui, T., Christophorou, M. A., Evan, G. I., Thomas-Tikhonenko, A., and Thompson, C. B. (2007). J Clin Invest 117, 326-36.
Bardeesy, N., and DePinho, R. A. (2002). Nat Rev Cancer 2, 897-909. Berry, D. L., and Baehrecke, E. H. (2007). Cell 131, 1137-48.
Bertucci, F., Salas, S., Eysteries, S., Nasser, V., Finetti, P., Ginestier, C., Charafe-Jauffret, E., Loriod, B., Bachelart, L., Montfort, J., Victorero, G., Viret, F., Ollendorff, V., Fert, V., Giovaninni, M., Delpero, J. R., Nguyen, C., Viens, P., Monges, G., Birnbaum, D., and Houlgatte, R. (2004). Onco gene 23, 1377-91.
Bjorkoy, G., Lamark, T., Brech, A., Outzen, H., Perander, M., Overvatn, A., Stenmark, H., and Johansen, T. (2005). J Cell Biol 171, 603-14. Brahimi-Horn, M. C., Chiche, J., and Pouyssegur, J. (2007). Curr Opin Cell Biol 19, 223-9.
Brech, A., Ahlquist, T., Lothe, R. A., and Stenmark, H. (2009). Mol. Oncol.
Bucci, C., Parton, R. G., Mather, I. H., Stunnenberg, H., Simons, K., Hoflack, B., and Zerial, M. (1992). Cell 70, 715-28.
Carlsson, S. R., Roth, J., Piller, F., and Fukuda, M. (1988). J Biol Chem 263, 18911-9.
Chen, X., and Wang, Z. (2001). EMBO Rep 2, 68-74.
Cuenco, G. M., and Ren, R. (2001). Oncogene 20, 8236-48.
D'Abaco, G. M., Whitehead, R. H., and Burgess, A. W. (1996). Mol Cell Biol 16, 884-91.
Degenhardt, K., Mathew, R., Beaudoin, B., Bray, K., Anderson, D., Chen, G., Mukherjee, C., Shi, Y., Gelinas, C., Fan, Y., Nelson, D. A., Jin, S., and White, E. (2006). Cancer Cell 10, 51-64.
Dinneen, J. L., and Ceresa, B. P. (2004). Exp Cell Res 294, 509-22.
Elenbaas, B., Spirio, L., Koerner, F., Fleming, M. D., Zimonjic, D. B., Donaher, J. L., Popescu, N.C., Hahn, W. C., and Weinberg, R. A. (2001). Genes Dev 15, 50-65.
Fan, C., Wang, W., Zhao, B., Zhang, S., and Miao, J. (2006). Bioorg Med Chem 14, 3218-22.
Fanidi, A., Harrington, E. A., and Evan, G. I. (1992). Nature 359, 554-6. Fearon, E. R., and Vogelstein, B. (1990). Cell 61, 759-67.
Fortunato, F., Burgers, H., Bergmann, F., Rieger, P., Buchler, M. W., Kroemer, G., and Werner, J. (2009). Gastroenterology 137, 350-60, 360 e1-5. Furuta, S., Hidaka, E., Ogata, A., Yokota, S., and Kamata, T. (2004). Oncogene 23, 3898-904.
Gutierrez, M. G., Munafo, D. B., Beron, W., and Colombo, M. I. (2004). J Cell Sci 117, 2687-97.
Hahn, W. C., Counter, C. M., Lundberg, A. S., Beijersbergen, R. L., Brooks, M. W., and Weinberg, R. A. (1999). Nature 400, 464-8.
Hanahan, D., and Weinberg, R. A. (2000). Cell 100, 57-70.
Hannon, G. J. (2002). Nature 418, 244-51.
Harrison, B., Kraus, M., Burch, L., Stevens, C., Craig, A., Gordon-Weeks, P., and Hupp, T. R. (2008). J Biol Chem 283, 9999-1 0014.
Hendrix, N. D., Wu, R., Kuick, R., Schwartz, D. R., Fearon, E. R., and Cho, K. R. (2006). Cancer Res 66, 1354-62.
Hezel, A. F., Kimmelman, A. C., Stanger, B. Z., Bardeesy, N., and Depinho, R. A. (2006). Genes Dev 20, 1218-49.
Houlden, H., King, R. H., Muddle, J. R., Warner, T. T., Reilly, M. M., Orrell, R. W., and Ginsberg, L. (2004). Ann Neurol 56, 586-90.
Hoyer-Hansen, M., Bastholm, L., Mathiasen, I. S., Elling, F., and Jaattela, M. (2005). Cell Death Differ 12, 1297-309.
Iacobuzio-Donahue, C. A., Maitra, A., Olsen, M., Lowe, A. W., van Heek, N. T., Rosty, C., Walter, K., Sato, N., Parker, A., Ashfaq, R., Jaffee, E., Ryu, B., Jones, J., Eshleman, J. R., Yeo, C. J., Cameron, J. L., Kern, S. E., Hruban, R. H., Brown, P. 0., and Goggins, M. (2003). Am J Pathol 162, 1151-62.
Ingvarsson, S. (1999). Semin Cancer Biol 9, 277-88.
Jager, S., Bucci, C., Tanida, I., Ueno, T., Kominami, E., Saftig, P., and Eskelinen, E. L. (2004). J Cell Sci 117, 4837-48.
Jones, S., Zhang, X., Parsons, D. W., Lin, J. C., Leary, R. J., Angenendt, P., Mankoo, P., Carter, H., Kamiyama, H., Jimeno, A., Hong, S. M., Fu, B., Lin, M. T., Calhoun, E. S., Kamiyama, M., Walter, K., Nikolskaya, T., Nikolsky, Y., Hartigan, J., Smith, D. R., Hidalgo, M., Leach, S. D., Klein, A. P., Jaffee, E. M., Goggins, M., Maitra, A., Iacobuzio-Donahue, C., Eshleman, J. R., Kern, S. E., Hruban, R. H., Karchin, R., Papadopoulos, N., Parmigiani, G., Vogelstein, B., Velculescu, V. E., and Kinzler, K. W. (2008). Science 321, 1801-6.
Jung, C. H., Jun, C. B., Ro, S. H., Kim, Y. M., Otto, N. M., Cao, J., Kundu, M., and Kim, D. H. (2009). Mol Biol Cell 20, 1992-2003.
Kabeya, Y., Mizushima, N., Ueno, T., Yamamoto, A., Kirisako, T., Noda, T., Kominami, E., Ohsumi, Y., and Yoshimori, T. (2000). EMBO J. 19, 5720-8. Kinzler, K. W., and Vogelstein, B. (1996). Cell 87, 159-70.
Kondo, Y., Kanzawa, T., Sawaya, R., and Kondo, S. (2005). Nat Rev Cancer 5, 726-34.
Land, H., Parada, L. F., and Weinberg, R. A. (1983a). Science 222, 771-8. Land, H., Parada, L. F., and Weinberg, R. A. (1983b). Nature 304, 596-602. Ledford, J. G., Kovarova, M., and Koller, B. H. (2007). JImmunol 178, 5132-43. Levine, B. (2007). Nature 446, 745-7.
Levine, B., and Klionsky, D. J. (2004). Dev Cell 6, 463-77.
Levine, B., and Kroemer, G. (2008). Cell 132, 27-42.
Liang, C., Feng, P., Ku, B., Dotan, I., Canaani, D., Oh, B. H., and Jung, J. U. (2006). Nat Cell Biol 8, 688-99.
Liang, X. H., Jackson, S., Seaman, M., Brown, K., Kempkes, B., Hibshoosh, H., and Levine, B. (1999). Nature 402, 672-6.
Lloyd, A. C., Obermuller, F., Staddon, S., Barth, C. F., McMahon, M., and Land, H. (1997). Genes Dev 11, 663-77.
Luhua, S., Ciftci-Yilmaz, S., Harper, J., Cushman, J., and Mittler, R. (2008). Plant Physiol 148, 280-92.
Lum, J. J., DeBerardinis, R. J., and Thompson, C. B. (2005). Nat Rev Mol Cell Biol 6, 439-48.
Maiuri, M. C., Tasdemir, E., Criollo, A., Morselli, E., Vicencio, J. M., Carnuccio, R., and Kroemer, G. (2009). Cell Death Differ 16, 87-93.
Maiuri, M. C., Zalckvar, E., Kimchi, A., and Kroemer, G. (2007). Nat Rev Mol Cell Biol 8, 741-52.
Maron, B. J., Roberts, W. C., Arad, M., Haas, T. S., Spirito, P., Wright, G. B., Almquist, A. K., Baffa, J. M., Saul, J. P., Ho, C. Y., Seidman, J., and Seidman, C. E. (2009). JAMA 301, 1253-9.
Mathew, R., Karp, C. M., Beaudoin, B., Vuong, N., Chen, G., Chen, H. Y., Bray, K., Reddy, A., Bhanot, G., Gelinas, C., Dipaola, R. S., Karantza Wadsworth, V., and White, E. (2009). Cell 137, 1062-75.
McMurray, H. R., Sampson, E. R., Compitello, G., Kinsey, C., Newman, L., Smith, B., Chen, S. R., Klebanov, L., Salzman, P., Yakovlev, A., and Land, H. (2008). Nature 453, 1112-6.
Meggouh, F., Bienfait, H. M., Weterman, M. A., de Visser, M., and Baas, F. (2006). Neurology 67, 1476-8.
Meresse, S., Gorvel, J. P., and Chavrier, P. (1995). J Cell Sci 108 (Pt 11), 3349-58.
Mizushima, N., Sugita, H., Yoshimori, T., and Ohsumi, Y. (1998). J Biol Chem 273, 33889-92.
Mizushima, N., Yamamoto, A., Hatano, M., Kobayashi, Y., Kabeya, Y., Suzuki, K., Tokuhisa, T., Ohsumi, Y., and Yoshimori, T. (2001). J CellBiol 152, 657-68.
Moore, P. S., Beghelli, S., Zamboni, G., and Scarpa, A. (2003). Mol Cancer 2, 7. Ohta, T., Arakawa, H., Futagami, F., Fushida, S., Kitagawa, H., Kayahara, M., Nagakawa, T., Miwa, K., Kurashima, K., Numata, M., Kitamura, Y., Terada, T., and Ohkuma, S. (1998). JPathol 185, 324-30.
Pankiv, S., Clausen, T. H., Lamark, T., Brech, A., Bruun, J. A., Outzen, H., Overvatn, A., Bjorkoy, G., and Johansen, T. (2007). J Biol Chem 282, 24131-45.
Pattingre, S., Bauvy, C., and Codogno, P. (2003). J Biol Chem 278, 16667-74. Qu, X., Yu, J., Bhagat, G., Furuya, N., Hibshoosh, H., Troxel, A., Rosen, J., Eskelinen, E. L., Mizushima, N., Ohsumi, Y., Cattoretti, G., and Levine, B. (2003). J Clin Invest 112, 1809-20.
Ramaswamy, S., Ross, K. N., Lander, E. S., and Golub, T. R. (2003). Nat Genet. 33, 49-54.
Rogulski, K., Li, Y., Rothermund, K., Pu, L., Watkins, S., Yi, F., and Prochownik, E. V. (2005). Oncogene 24, 7524-41.
Roper, E., Weinberg, W., Watt, F. M., and Land, H. (2001). EMBO Rep 2, 145-50.
Ruley, H. E. (1983). Nature 304, 602-6.
Saftig, P., Beertsen, W., and Eskelinen, E. L. (2008). Autophagy 4, 510-2.
Sanchez-Carbayo, M., Socci, N. D., Lozano, J., Saint, F., and Cordon-Cardo, C. (2006). J Clin Oncol 24, 778-89.
Schulze, A., Lehmann, K., Jefferies, H. B., McMahon, M., and Downward, J. (2001). Genes Dev 15, 981-94.
Settembre, C., Fraldi, A., Jahreiss, L., Spampanato, C., Venturi, C., Medina, D., de Pablo, R., Tacchetti, C., Rubinsztein, D.C., and Ballabio, A. (2008). Hum Mol Genet. 17, 119-29.
Song, W. Y., Martinoia, E., Lee, J., Kim, D., Kim, D. Y., Vogt, E., Shim, D., Choi, K. S., Hwang, I., and Lee, Y. (2004). PlantPhysiol 135, 1027-39.
Stypmann, J., Janssen, P. M., Prestle, J., Engelen, M. A., Kogler, H., Lullmann-Rauch, R., Eckardt, L., von Figura, K., Landgrebe, J., Mleczko, A., and Saftig, P. (2006). Basic Res Cardiol 101, 281-91.
Sun, L., Hui, A. M., Su, Q., Vortmeyer, A., Kotliarov, Y., Pastorino, S., Passaniti, A., Menon, J., Walling, J., Bailey, R., Rosenblum, M., Mikkelsen, T., and Fine, H. A. (2006). Cancer Cell 9, 287-300.
Suzuki, C., Isaka, Y., Takabatake, Y., Tanaka, H., Koike, M., Shibata, M., Uchiyama, Y., Takahara, S., and Imai, E. (2008). Biochem Biophys Res Commun 368, 100-6.
Tanaka, Y., Guhde, G., Suter, A., Eskelinen, E. L., Hartmann, D., LullmannRauch, R., Janssen, P. M., Blanz, J., von Figura, K., and Saftig, P. (2000). Nature 406, 902-6.
Tanida, I., Nishitani, T., Nemoto, T., Ueno, T., and Kominami, E. (2002). Biochem Biophys Res Commun 296, 1164-70.
Tasdemir, E., Maiuri, M. C., Galluzzi, L., Vitale, I., Djavaheri-Mergny, M., D'Amelio, M., Criollo, A., Morselli, E., Zhu, C., Harper, F., Nannmark, U., Samara, C., Pinton, P., Vicencio, J. M., Carnuccio, R., Moll, U. M., Madeo, F., Paterlini-Brechot, P., Rizzuto, R., Szabadkai, G., Pierron, G., Blomgren, K., Tavernarakis, N., Codogno, P., Cecconi, F., and Kroemer, G. (2008). Nat Cell Biol 10, 676-87.
Tsukada, M., and Ohsumi, Y. (1993). FEBS Lett 333, 169-74.
Tsuneoka, M., Umata, T., Kimura, H., Koda, Y., Nakajima, M., Kosai, K., Takahashi, T., Takahashi, Y., and Yamamoto, A. (2003). Cell Struct Funct 28, 195-204.
Tunon, T., Guerrero, D., Urchaga, A., Nishino, I., Ayuso, T., Matsuda, Y., Caballero, M. C., Berjon, J., and Imizcoz, M. A. (2008). Neuromuscul Disord 18, 167-74.
van't Veer, L. J., Dai, H., van de Vijver, M. J., He, Y. D., Hart, A. A., Mao, M., Peterse, H. L., van der Kooy, K., Marton, M. J., Witteveen, A. T., Schreiber, G. J., Kerkhoven, R. M., Roberts, C., Linsley, P. S., Bernards, R., and Friend, S. H. (2002). Nature 415, 530-6.
Verhoeven, K., De Jonghe, P., Coen, K., Verpoorten, N., Auer-Grumbach, M., Kwon, J. M., FitzPatrick, D., Schmedding, E., De Vriendt, E., Jacobs, A., Van Gerwen, V., Wagner, K., Hartung, H. P., and Timmerman, V. (2003). Am J Hum Genet. 72, 722-7.
Witz, I. P. (2008). Adv Cancer Res 100, 203-29.
Wood, L. D., Parsons, D. W., Jones, S., Lin, J., Sjoblom, T., Leary, R. J., Shen, D., Boca, S. M., Barber, T., Ptak, J., Silliman, N., Szabo, S., Derso, Z., Ustyanksky, V., Nikolskaya, T., Nikolsky, Y., Karchin, R., Wilson, P. A., Kaminker, J. S., Zhang, Z., Croshaw, R., Willis, J., Dawson, D., Shipitsin, M., Willson, J. K., Sukumar, S., Polyak, K., Park, B. H., Pethiyagoda, C. L., Pant, P. V., Ballinger, D. G., Sparks, A. B., Hartigan, J., Smith, D. R., Suh, E., Papadopoulos, N., Buckhaults, P., Markowitz, S. D., Parmigiani, G., Kinzler, K. W., Velculescu, V. E., and Vogelstein, B. (2007). Science 318, 1108-13.
Xia, M., and Land, H. (2007). Nat Struct Mol Biol 14, 215-23.
Yue, Z., Jin, S., Yang, C., Levine, A. J., and Heintz, N. (2003). Proc Natl Acad Sci USA 100, 15077-82.
Zhao, R., Gish, K., Murphy, M., Yin, Y., Notterman, D., Hoffman, W. H., Tom, E., Mack, D. H., and Levine, A. J. (2000). Genes Dev 14, 981-93.
Zheng, Y., Zhao, Y. L., Deng, X., Yang, S., Mao, Y., Li, Z., Jiang, P., Zhao, X., and Wei, Y. (2009). Cancer Invest 27, 286-92.

What is claimed is:

1. A method of treating a cancer in a subject comprising administering to the subject an agent that modulates the rate of autophagy in the cancer.

2. The method of claim 1, wherein the agent increases the rate of autophagy.

3. The method of claim 2, wherein the agent is a protein or nucleic acid that encodes a protein that activates autophagy.

4. The method of claim 3, wherein the protein or nucleic acid that encodes a protein that activates autophage is selected from the group consisting of Plac8, ATG1, ATG2, ATG3, ATG4, ATG5, ATG6, ATG7, ATG8, ATG8, ATG10, ATG11, ATG12, ATG13, ATG14, ATG15, ATG16, ATG17, ATG18, ATG19, ATG20, ATG21, ATG22, ATG23, ATG24, ATG25, ATG26, ATG27, ATG28, ATG29, ATG30, ATG31, ATG101, LC3, RAB7, VPS15, VPS35, UVRAG, Beclin1, BCL2, BCL-XL, ULK1 ULK2, ULK3, ULK4, DapK1, FIP200, TSC1, TSC2, AMPK, Redd1, CAMKKbeta, LKB, MO25, STRAD, and PTEN.

5. The method of claim 2, wherein the agent is a siRNA or small molecule which binds to a protein that inhibits authophagy or nucleic acid that encodes said protein.

6. The method of claim 5, wherein the protein is selected from the group consisting of mTOR, Raptor, Deptor, Rictor, Protor, PRAS40, LST8, Rheb, RAG A, RAG B, RAG C, RAG D, AKT, PDK1, PI3K, IRS1, Insulin/IGF1 receptor, ERK, MEK, RAF, SIN1, MAP4K3, SLC7A5, and SLC3A2.

7. The method of claim 1, wherein the agent decrease the rate of autophagy.

8. The method of claim 7, wherein the agent is a protein, peptide, or nucleic acid that encodes a protein or peptide, wherein the agent acts as a competitive inhibitor of an protein that activates autophagy or its overexpression has a tumor inhibitory effect.

9. The method of claim 8, wherein the competitive inhibitor competes with a protein selected from the group consisting of Plac8, ATG1, ATG2, ATG3, ATG4, ATG5, ATG6, ATG7, ATG8, ATG8, ATG10, ATG11, ATG12, ATG13, ATG14, ATG15, ATG16, ATG17, ATG18, ATG19, ATG20, ATG21, ATG22, ATG23, ATG24, ATG25, ATG26, ATG27, ATG28, ATG29, ATG30, ATG31, ATG101, LC3, RAB7, VPS15, VPS35, UVRAG, Beclin1, Rab40b, BCL2, BCL-XL, ULK1 ULK2, ULK3, ULK4, FIP200, TSC1, TSC2, AMPK, Redd1, CAMKKbeta, LKB, MO25, STRAD, and PTEN.

10. The method of claim 5, wherein the agent is a siRNA or small molecule which binds to a protein that activates autophagy or nucleic acid that encodes said protein.

11. The method of claim 10, wherein the protein is selected from the group consisting of Plac8, ATG1, ATG2, ATG3, ATG4, ATG5, ATG6, ATG7, ATG8, ATG8, ATG10, ATG11, ATG12, ATG13, ATG14, ATG15, ATG16, ATG17, ATG18, ATG19, ATG20, ATG21, ATG22, ATG23, ATG24, ATG25, ATG26, ATG27, ATG28, ATG29, ATG30, ATG31, ATG101, LC3, RAB7, VPS15, VPS35, UVRAG, Beclin1, Rab40b, BCL2, BCL-XL, ULK1 ULK2, ULK3, ULK4, Plac8, FIP200, TSC1, TSC2, AMPK, Redd1, CAMKKbeta, LKB, MO25, STRAD, and PTEN.

12. The method of claim 11, wherein the agent binds to Plac8.

13. The method of claim 10, wherein the small molecule is selected from the group consisting of Chloroquine and Bafilomycin A1.

14. The method of claim 5, wherein the agent is a protein or nucleic acid that encodes a protein that inhibits autophagy.

15. The method of claim 10, wherein the agent is selected from the group consisting of mTOR, Raptor, Deptor, Rictor, Protor, PRAS40, LST8, Rheb, RAG A, RAG B, RAG C, RAG D, AKT, PDK1, PI3K, IRS1, Insulin/IGF1 receptor, ERK, MEK, RAF, SIN1, MAP4K3, SLC7A5, and SLC3A2.

16. The method of claim 1, wherein the rate of autophagy is modulated in a direction that approaches the rate of autophagy in a nontransformed cell.

17. The method of claim 1, wherein the rate of autophagy is modulated further in a direction away from the rate of autophagy in a nontransformed cell.

18. The method of claim 1, wherein the cancer is selected from the group of cancers consisting of lymphoma, B cell lymphoma, T cell lymphoma, mycosis fungoides, Hodgkin's Disease, leukemias, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, gastric cancer, colon cancer, colorectal adenocarcinoma, pancreatic adenocarcinoma, cervical cancer, cervical carcinoma, breast cancer, and epithelial cancer, bone cancers, renal cancer, bladder cancer, genitourinary cancer, esophageal carcinoma, large bowel cancer, metastatic cancers hematopoietic cancers, sarcomas, Ewing's sarcoma, synovial cancer, soft tissue cancers; and testicular cancer.

19. A method of screening for an agent that treats cancer comprising measuring the rate of autophagy in a cancer cell and a non-cancerous control cell, determining of the rate of autophagy in the cancer cell is increased or decreased relative to the rate of autophagy in the control cell, contacting a cancer cell with the agent, and measuring the rate of autophagy, wherein an agent that modulates the rate of autophagy in the cancer cell in a direction towards the rate of autophagy in the control cell indicates an agent that can treat cancer.

20. The method of claim 19, wherein the agent increases the rate of autophagy.

21. The method of claim 20, wherein the agent is a protein or nucleic acid that encodes a protein that activates autophagy.

22. The method of claim 19, wherein the agent decrease the rate of autophagy.

23. The method of claim 22, wherein the agent is a protein, peptide, or nucleic acid that encodes a protein or peptide, wherein the agent acts as a competitive inhibitor of an protein that activates autophagy.

24. The method of claim 23, wherein the agent is a non-functional Plac8.

25. The method of claim 22, wherein the agent is a siRNA or small molecule which binds to a protein that activates autophagy.

26. The method of claim 22, wherein the agent siRNA is a shRNA that specifically binds to Plac8.

27. The method of claim 22, wherein the agent is a protein or nucleic acid that encodes a protein that inhibits autophagy.

28. The method of claim 19, wherein the cancer is selected from the group consisting of lymphoma, B cell lymphoma, T cell lymphoma, mycosis fungoides, Hodgkin's Disease, leukemias, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, gastric cancer, colon cancer, colorectal adenocarcinoma, pancreatic adenocarcinoma, cervical cancer, cervical carcinoma, breast cancer, and epithelial cancer, bone cancers, renal cancer, bladder cancer, genitourinary cancer, esophageal carcinoma, large bowel cancer, metastatic cancers hematopoietic cancers, sarcomas, Ewing's sarcoma, synovial cancer, soft tissue cancers; and testicular cancer.
